(19) United States
(12) Patent Application Publication
(10) Pub. No.: US 2024/0335522 A1
(43) Pub. Date: Oct. 10, 2024

(54) MULTIVALENT PAN-INFLUENZA VACCINE

(71) Applicant: Najít Technologies, Inc., Beaverton, OR (US)

(72) Inventors: Ian J. AMANNA, Hillsboro, OR (US); Arpita RAY, Portland, OR (US)

(21) Appl. No.: 18/292,873

(22) PCT Filed: Jul. 29, 2022
(86) PCT No.: PCT/US2022/074356

§ 371 Date: Jan. 26, 2024

Related U.S. Application Data
(60) Provisional application No. 63/230,643, filed on Aug. 6, 2021.

(51) Int. Cl. A61K 39/145; A61K 39/00; C12N 7/00

(52) U.S. Cl. CPC A61K 39/145; C12N 7/00; A61K 2039/70; C12N 2760/16134; C12N 2760/16234

(57) ABSTRACT

Provided are highly immunogenic multivalent pan-influenza vaccines, comprising a viral haemagglutinin (HA) protein, or HA1-containing portion thereof, of/corresponding to a virus strain from each of any three of, or from all four of component virus strain groups (H1-CVG1-H1-CVG-4) as defined herein. Additionally provided are highly immunogenic multivalent pan-influenza vaccine, comprising a viral hacmagglutinin (HA) protein, or HA1-containing portion thereof, of/corresponding to a virus strain from each of any three of, or from all four of component virus strain groups (H3-CVG-1-H3-CVG-4) as defined herein. Further provided are highly immunogenic multivalent pan-influenza vaccine, comprising a viral haemagglutinin (HA) protein, or HA1-containing portion thereof, of/corresponding to a virus strain from each of two component virus strain groups Influenza B-CVG-1 and Influenza B-CVG-2 as defined herein. Yet further provided are methods for making the immunogenic vaccine compositions, and methods for eliciting an immune response, comprising administering the immunogenic vaccine compositions.

### CROSS-REFERENCE TO RELATED APPLICATION

This application claims priority to U.S. Patent Application No. 63/230,643, filed Aug. 6, 2021, entitled “MULTIVALENT PAN-INFLUENZA VACCINE”, which is hereby incorporated herein by reference in its entirety and for all purposes.

### STATEMENT REGARDING FEDERALLY SPONSORED RESEARCH

This work was supported at least in part by CDC Grant No. R43IP001130 and NIH Contract No. 75N93019C00050, and the United States government therefore has certain rights.

### SEQUENCE LISTING

A Sequence Listing (ST.26), comprising 556 SEQ ID NOS, has been provided in computer readable form (.xml) as part of this application, and is incorporated by reference herein in its entirety.

### FIELD OF THE INVENTION

Aspects of the present invention relate generally to influenza vaccine compositions and methods for making same, including more particularly to multivalent (e.g., divalent, trivalent, tetravalent, etc.) influenza A and B vaccine compositions and methods for making same, including even more particularly to: multivalent pan-influenza A vaccine compositions comprising a viral haemagglutinin (HA) protein, or HA1-containing portion thereof, of or corresponding to at least one virus strain from each of any three of, or from all four of component H1N1 virus groups H1-CVG-1-H1-CVG-4; to multivalent pan-influenza A vaccine compositions comprising a viral haemagglutinin (HA) protein, or HA1-containing portion thereof, of or corresponding to at least one virus strain from each of any three of, or from all four of component H3N2 virus groups H3-CVG-1-H3-CVG-); to multivalent pan-influenza B vaccine compositions comprising a viral haemagglutinin (HA) protein, or HA1-containing portion thereof, of or corresponding to at least one virus strain from each of two component B virus groups Influenza B-CVG-1 and Influenza B-CVG-2; and to methods for making the pan-influenza A and B vaccine compositions. Additional aspects relate to eliciting an immune response in a subject by administering the vaccine compositions.

### BACKGROUND

Influenza, commonly known as “the flu,” is an infectious disease caused by an influenza virus, RNA viruses that make up three of the five genera of the family Orthomyxoviridae. Influenza spreads around the world in a yearly outbreak, resulting in about three to five million cases of severe illness and about 250,000 to 500,000 deaths.

Vaccines (e.g., inactivated vaccines, etc.) represent a critical component of the health care system for both human and veterinary fields of medicine. Despite more than 70 years of vaccine research and development, however, influenza remains a pressing public health concern. Although multiple subtypes of influenza A have been identified, H1N1 and H3N2 are the only influenza A strains currently circulating in human populations. Estimates within the US suggest that seasonal influenza leads to more than 200,000 hospitalizations each year (Thompson, W. W., et al., Influenza-associated hospitalizations in the United States. JAMA, 2004. 292 (11): p. 1333-40), demonstrating a sustained, high-level of morbidity. Influenza-associated mortality also remains high, with over 20,000 deaths per year, particularly among the elderly (CDC, Estimates of deaths associated with seasonal influenza—United States, 1976-2007. MMWR Morb Mortal Wkly Rep, 2010. 59 (33): p. 1057-62; Matias, G., et al., Estimates of mortality attributable to influenza and RSV in the United States during 1997-2009 by influenza type or subtype, age, cause of death, and risk status. Influenza Other Respir Viruses, 2014. 8 (5): p. 507-15). To combat the threat posed by influenza, vaccination campaigns have been widely implemented, with the US recommending routine annual vaccination for all persons aged 6 months and older. Licensed vaccine strategies include live-attenuated, split-inactivated, and recombinant protein approaches formulated on a seasonal basis. Notwithstanding a long history of development and implementation, these current vaccine approaches remain largely ineffective at preventing disease (Osterholm, M. T., et al., Efficacy and effectiveness of influenza vaccines: a systematic review and meta-analysis. Lancet Infect Dis, 2012. 12 (1): p. 36-44). For example, the 2017-18 influenza season was particularly challenging, with high disease burden and vaccine efficacy estimates as low as 25-36% (Flannery, B., et al., Interim Estimates of 2017-18 Seasonal Influenza Vaccine Effectiveness-United States, February 2018. MMWR Morb Mortal Wkly Rep, 2018. 67 (6): p. 180-185). In addition to limited overall efficacy, recent clinical studies have demonstrated rapid waning of immunity (Young, B., et al., Do antibody responses to the influenza vaccine persist year-round in the elderly? A systematic review and meta-analysis. Vaccine, 2017. 35 (2): p. 212-221) and loss of protective efficacy during a single influenza season (Radin, J. M., et al., Influenza vaccine effectiveness: Maintained protection throughout the duration of influenza seasons 2010-2011 through 2013-2014. Vaccine, 2016. 34 (33): p. 3907-12; Ferdinands, J. M., et al., Intraseason waning of influenza vaccine protection: Evidence from the US Influenza Vaccine Effectiveness Network, 2011-12 through 2014-15. Clin Infect Dis, 2017. 64 (5): p. 544-550), even in the absence of antigenic drift (Jimenez-Jorge, S., et al., Effectiveness of influenza vaccine against laboratory-confirmed influenza, in the late 2011-2012 season in Spain, among population targeted for vaccination. BMC Infect Dis, 2013. 13: p. 441). Given these observations of moderate to poor overall efficacy with limited durability, there is a significant unmet need to produce more immunogenic, broader spectrum vaccines against influenza.

### SUMMARY OF EXEMPLARY ASPECTS OF THE INVENTION

Embodiments of the disclosure can be described in view of the following clauses:

1. A multivalent pan-influenza vaccine, comprising a viral haemagglutinin (HA) protein, or HA1-containing portion thereof, and/or comprising a nucleic acid encoding the HA protein or the HA1-containing portion thereof, of or corresponding to a virus strain from each of any three of, or from all four of component virus strain groups H1-CVG1-H1-CVG-4, wherein: H1-CVG-1 comprises H1N1 virus strains having either (i) a conjoined Sa, Sb, Ca1, Ca2, and Cb HA antigenic sites amino acid (aa) sequence having at least 82% sequence identity with SEQ ID NO:85, and/or (ii) a HA1 Globular Head Region aa sequence having at least 91% sequence identity with SEQ ID NO: 173; H1-CVG-2 comprises H1N1 virus strains having either (i) a conjoined Sa, Sb, Ca1, Ca2, and Cb HA antigenic sites aa sequence having at least 90% sequence identity with SEQ ID NO:86, and/or (ii) a HA1 Globular Head Region aa sequence having at least 96% sequence identity with SEQ ID NO:174; H1-CVG-3 comprises H1N1 virus strains having either (i) a conjoined Sa, Sb, Ca1, Ca2, and Cb HA antigenic sites aa sequence having at least 92% sequence identity with SEQ ID NO:87, and/or (ii) a HA1 Globular Head Region aa sequence having at least 93% sequence identity with SEQ ID NO:175; and H1-CVG-4 comprises H1N1 virus strains having either (i) a conjoined Sa, Sb, Ca1, Ca2, and Cb HA antigenic sites aa sequence having at least 88% sequence identity with SEQ ID NO:88, and/or (ii) a HA1 Globular Head Region aa sequence having at least 96% sequence identity with SEQ ID NO: 176.

2. The vaccine of clause 1, wherein: the virus strain from H1-CVG-1 is WS33 having HA SEQ ID NO: 177, and/or is PR8 having HA SEQ ID NO: 178; and/or the virus strain from H1-CVG-2 is FM47 having HA SEQ ID NO:179, and/or is USSR77 having HA SEQ ID NO:180; and/or the virus strain from H1-CVG-3 is BR07 having HA SEQ ID NO: 182, and/or is SI06 having HA SEQ ID NO: 181; and/or the virus strain from H1-CVG-4 is NEB 19 having HA SEQ ID NO: 184, and/or is MCH15 having HA SEQ ID NO:183.

3. The vaccine of clause 2, wherein: the virus strain from H1-CVG-1 is WS33 having HA SEQ ID NO: 177, and/or is PR8 having HA SEQ ID NO:178; and the virus strain from H1-CVG-2 is FM47 having HA SEQ ID NO:179, and/or is USSR77 having HA SEQ ID NO: 180; and the virus strain from H1-CVG-3 is BR07 having HA SEQ ID NO:182, and/or is SI06 having HA SEQ ID NO:181; and the virus strain from H1-CVG-4 is NEB19 having HA SEQ ID NO: 184, and/or is MCH15 having HA SEQ ID NO:183.

4. The vaccine of clause 2, wherein: the virus strain from H1-CVG-1 is WS33; and/or the virus strain from H1-CVG-2 is FM47; and/or the virus strain from H1-CVG- is BR07; and/or the virus strain from H1-CVG-4 is NEB19.

5. The vaccine of clause 4, wherein: the virus strain from H1-CVG-1 is WS33; and the virus strain from H1-CVG-2 is FM47; and the virus strain from H1-CVG-3 is BR07; and the virus strain from H1-CVG-4 is NEB19.

6. The vaccine of any one of clauses 1-5, wherein: the Globular Head Region of the virus strain from H1-CVG-1 comprises one or more predicted and/or confirmed N-linked glycosylation site(s) (NLGs); and/or the Globular Head Region of the virus strain from H1-CVG-2 comprises one or more predicted and/or confirmed NLGs; and/or the Globular Head Region of the virus strain from H1-CVG-3 comprises one or more predicted and/or confirmed NLGs; and/or the Globular Head Region of the virus strain from H1-CVG-4 comprises one or more predicted and/or confirmed NLGs (NLGs).

7. The vaccine of clause 6, wherein: the Globular Head Region of the virus strain from H1-CVG-1 comprises one or more predicted and/or confirmed NLGs; and the Globular Head Region of the virus strain from H1-CVG-2 comprises one or more predicted and/or confirmed NLGs; and the Globular Head Region of the virus strain from H1-CVG-3 comprises one or more predicted and/or confirmed NLGs; and the Globular Head Region of the virus strain from H1-CVG-4 comprises one or more predicted and/or confirmed NLGs (NLGs).

8. The vaccine of clause 6, wherein: the Globular Head Region of the virus strain from H1-CVG-1 comprises at least two predicted and/or confirmed NLGs; and/or the Globular Head Region of the virus strain from H1-CVG-2 comprises at least three predicted and/or confirmed NLGs; and/or the Globular Head Region of the virus strain from H1-CVG-3 comprises at least four predicted and/or confirmed NLGs; and/or the Globular Head Region of the virus strain from H1-CVG-4 comprises at least two predicted and/or confirmed NLGs.

9. The vaccine of clause 8, wherein: the Globular Head Region of the virus strain from H1-CVG-1 comprises at least two predicted and/or confirmed NLGs; and the Globular Head Region of the virus strain from H1-CVG-2 comprises at least three predicted and/or confirmed NLGs; and the Globular Head Region of the virus strain from H1-CVG-3 comprises at least four predicted and/or confirmed NLGs; and the Globular Head Region of the virus strain from H1-CVG-4 comprises at least two predicted and/or confirmed NLGs.

10. The vaccine of any one of clauses 1-9, wherein the HA protein or HA1-containing portion thereof from each of the any three of, or the four component virus groups are present as one or more components that can be administered together, or sequentially.

11. The vaccine of clause 10, wherein the HA protein or HA1-containing portion thereof from the three or the four component virus groups are combined in a multivalent vaccine composition for coadministration.

12. The vaccine of any one of clauses 1-11, further comprising an adjuvant, and/or a pharmaceutically acceptable carrier, diluent, or excipient.

13. The vaccine of clause 12, wherein the adjuvant comprises one or more aluminum salts.

14. The vaccine of any one of clauses 1-13, wherein, independently with respect to each of the three or the four component virus groups, the HA protein(s) or the HA1-containing portion(s) thereof is present as one or more of: a component of an inactivated virus or component thereof; a component of a recombinant virus or component thererof; a recombinant HA or component thereof; and/or a component of a nanoparticle vaccine delivery platform/composition (e.g., including liposomes).

15. The vaccine of clause 14, wherein, independently with respect to each of the any three of, or the four component virus groups, the HA protein(s) or the HA1-containing portion(s) thereof is present as one or more of: a component of an inactivated virus or component thereof; and/or as a recombinant HA or component thereof.

16. The vaccine of clause 15, wherein, with respect to the any three of, or the four component virus groups, the HA protein(s) or the HA1-containing portion(s) thereof is present as a component of an inactivated virus or component thereof, or as a recombinant HA or component thereof.

17. The vaccine of clause 16, wherein, with respect to the any three of, or the four component virus groups, the HA protein(s) or the HA1-containing portion(s) thereof is present as a component of an inactivated virus or component thereof.

18. The vaccine of any one of clauses 1-17 or 70, wherein independently for each of the any three of, or the four component virus groups, the vaccine comprises the HA protein(s), or the HA1-containing portion(s) thereof, of only one viral strain per group.

19. A method of eliciting an immune response, comprising administering an immunogenic vaccine composition according to any one of claims 1-18 to a subject, thereby eliciting in the subject an immune response against influenza.

20. The method of clause 19, wherein eliciting the immune response comprises eliciting an H1N1 influenza virus-specific immune response, and/or a pan-H1N1 influenza virus-specific immune response.

21. The method of clause 20, wherein eliciting the immune response additionally comprises eliciting an immune response to at least one non-H1N1 vaccine strain.

22. The method of any one of clauses 19-21, wherein the immune response comprises one or more of an antibody, a B cell, and/or a T cell response.

23. The method of any one of clauses 19-22, wherein administration comprises administering the vaccine in one or more components administered together, or sequentially

24. A multivalent pan-influenza vaccine, comprising a viral haemagglutinin (HA) protein, or HA1-containing portion thereof, and/or comprising a nucleic acid encoding the HA protein or the HA1-containing portion thereof, of or corresponding to a virus strain from each of any three of, or from all four of component virus strain groups H3-CVG-1-H3-CVG-4, wherein: H3-CVG-1 comprises H3N2 virus strains having either (i) a conjoined A, B, C, D, and E HA antigenic sites amino acid (aa) sequence having at least 88% sequence identity with SEQ ID NO:268, and/or (ii) a HA1 Globular Head Region aa sequence having at least 93% sequence identity with SEQ ID NO:355; H3-CVG-2 comprises H3N2 virus strains having either (i) a conjoined A, B, C, D, and E HA antigenic sites aa sequence having at least 95% sequence identity with SEQ ID NO:269, and/or (ii) a HA1 Globular Head Region aa sequence having at least 98% sequence identity with SEQ ID NO:356; H3-CVG-3 comprises H3N2 virus strains having either (i) a conjoined A, B, C, D, and E HA antigenic sites aa sequence having at least 93% sequence identity with SEQ ID NO:270, and/or (ii) a HA1 Globular Head Region aa sequence having at least 96% sequence identity with SEQ ID NO: 357; and H3-CVG-4 comprises H3N2 virus strains having either (i) a conjoined A, B, C, D, and E HA antigenic sites aa sequence having at least 89% sequence identity with SEQ ID NO: 271, and/or (ii) a HA1 Globular Head Region aa sequence having at least 95% sequence identity with SEQ ID NO:358.

25. The vaccine of clause 24, wherein: the virus strain from H3-CVG-1 is TX77 having HA SEQ ID NO:359, and/or is BK79 having HA SEQ ID NO:360; and/or the virus strain from H3-CVG-2 is BE89 having HA SEQ ID NO:361, and/or is BE92 having HA SEQ ID NO: 362; and/or the virus strain from H3-CVG-3 is FU02 having HA SEQ ID NO:364, and/or is NE03 having HA SEQ ID NO:363; and/or the virus strain from H3-CVG-4 is HK19 having HA SEQ ID NO:365, and/or is CB20 having HA SEQ ID NO:366.

26. The vaccine of clause 25, wherein: the virus strain from H3-CVG-1 is TX77 having HA SEQ ID NO:359, and/or is BK79 having HA SEQ ID NO:360; and the virus strain from H3-CVG-2 is BE89 having HA SEQ ID NO:361, and/or is BE92 having HA SEQ ID NO: 362; and the virus strain from H3-CVG-3 is FU02 having HA SEQ ID NO:364, and/or is NE03 having HA SEQ ID NO:363; and the virus strain from H3-CVG-4 is HK19 having HA SEQ ID NO:365, and/or is CB20 having HA SEQ ID NO:366.

27. The vaccine of clause 25, wherein: the virus strain from H3-CVG-1 is TX77; and/or the virus strain from H3-CVG-2 is BE89; and/or the virus strain from H3-CVG-is FU02; and/or the virus strain from H3-CVG-4 is HK19.

28. The vaccine of clause 27, wherein: the virus strain from H3-CVG-1 is TX77; and the virus strain from H3-CVG-2 is BE89; and the virus strain from H3-CVG-3 is FU02; and the virus strain from H3-CVG-4 is HK19.

29. The vaccine of any one of clauses 24-28, wherein: the Globular Head Region of the virus strain from H3-CVG-1 comprises one or more predicted and/or confirmed N-linked glycosylation site(s) (NLGs); and/or the Globular Head Region of the virus strain from H3-CVG-2 comprises one or more predicted and/or confirmed NLGs; and/or the Globular Head Region of the virus strain from H3-CVG-3 comprises one or more predicted and/or confirmed NLGs; and/or the Globular Head Region of the virus strain from H3-CVG-4 comprises one or more predicted and/or confirmed NLGs (NLGs).

30. The vaccine of clause 29, wherein: the Globular Head Region of the virus strain from H3-CVG-1 comprises one or more predicted and/or confirmed NLGs; and the Globular Head Region of the virus strain from H3-CVG-2 comprises one or more predicted and/or confirmed NLGs; and the Globular Head Region of the virus strain from H3-CVG-3 comprises one or more predicted and/or confirmed NLGs; and the Globular Head Region of the virus strain from H3-CVG-4 comprises one or more predicted and/or confirmed NLGs (NLGs).

31. The vaccine of clause 29, wherein: the Globular Head Region of the virus strain from H3-CVG-1 comprises at least three predicted and/or confirmed NLGs; and/or the Globular Head Region of the virus strain from H3-CVG-2 comprises at least four predicted and/or confirmed NLGs; and/or the Globular Head Region of the virus strain from H3-CVG-3 comprises at least six predicted and/or confirmed NLGs; and/or the Globular Head Region of the virus strain from H3-CVG-4 comprises at least four predicted and/or confirmed NLGs.

32. The vaccine of clause 31, wherein: the Globular Head Region of the virus strain from H3-CVG-1 comprises at least three predicted and/or confirmed NLGs; and the Globular Head Region of the virus strain from H3-CVG-2 comprises at least four predicted and/or confirmed NLGs; and the Globular Head Region of the virus strain from H3-CVG-3 comprises at least six predicted and/or confirmed NLGs; and the Globular Head Region of the virus strain from H3-CVG-4 comprises at least four predicted and/or confirmed NLGs.

33. The vaccine of any one of clauses 24-32, wherein the HA proteins or HA1-containing portions thereof from the any three of, or the four component virus groups are present in one or more components that can be administered together, or sequentially.

34. The vaccine of clause 33, wherein the HA proteins or HA1-containing portions thereof from the any three of, or the four component virus groups are combined in a multivalent vaccine composition for coadministration.

35. The vaccine of any one of clauses 24-34, further comprising an adjuvant, and/or a pharmaceutically acceptable carrier, diluent, or excipient.

36. The vaccine of clause 35, wherein the adjuvant comprises one or more aluminum salts.

37. The vaccine of any one of clauses 24-36, wherein, independently with respect to each of the any three of, or the four component virus groups, the HA protein(s) or the HA1-containing portion(s) thereof is present as one or more of: a component of an inactivated virus component thereof; a component of a recombinant virus or component thereof; a recombinant HA or component thereof; and/or a component of a nanoparticle vaccine delivery platform/composition (e.g., including liposomes).

38. The vaccine of clause 37, wherein, independently with respect to each of the any three of, or the four component virus groups, the HA protein(s) or the HA1-containing portion(s) thereof is present as one or more of: a component of an inactivated virus or component thereof; and/or as a recombinant HA or component thereof.

39. The vaccine of clause 38, wherein, with respect to the any three of, or the four component virus groups, the HA protein(s) or the HA1-containing portion(s) thereof is present as a component of an inactivated virus or component thereof, or as a recombinant HA or component thereof.

40. The vaccine of clause 39, wherein, with respect to the any three of, or the four component virus groups, the HA protein(s) or the HA1-containing portion(s) thereof is present as a component of an inactivated virus or component thereof.

41. The vaccine of any one of clause s 24-40 or 71, wherein for each of the any three of, or the four component virus groups, the vaccine comprises the HA protein(s), or the HA1-containing portion(s) thereof, of only one viral strain for each group.

42. A method of eliciting an immune response, comprising administering an immunogenic vaccine composition according to any one of claims 24-41 to a subject, thereby eliciting in the subject an immune response against influenza.

43. The method of clause 42, wherein eliciting the immune response comprises eliciting an H3N2 influenza virus-specific immune response, and/or a pan-H3N2 influenza virus-specific immune response.

44. The method of clause 43, wherein eliciting the immune response additionally comprises eliciting an immune response to at least one non-H3N2 vaccine strain.

45. The method of any one of clauses 42-44, wherein the immune response comprises one or more of an antibody, a B cell, and/or a T cell response.

46. The method of any one of clauses 24-45, wherein administration comprises administering the vaccine in one or more components administered together, or sequentially.

47. A multivalent pan-influenza vaccine, comprising a viral haemagglutinin (HA) protein, or HA1-containing portion thereof, and/or comprising a nucleic acid encoding the HA protein or the HA1-containing portion thereof, of or corresponding to a virus strain from each of two component virus strain groups Influenza B-CVG-1 and Influenza B-CVG-2, wherein: Influenza B-CVG-1 comprises Influenza B virus strains having either (i) a conjoined 120 loop, 150 loop, 160 loop, 190 helix, and 230 region HA antigenic sites amino acid (aa) sequence having at least 94% sequence identity with SEQ ID NO:458, and/or (ii) a HA1 Globular Head Region aa sequence having at least 98% sequence identity with SEQ ID NO: 551; and Influenza B-CVG-2 comprises Influenza B virus strains having either (i) a conjoined 120 loop, 150 loop, 160 loop, 190 helix, and 230 region HA antigenic sites aa sequence having at least 94% sequence identity with SEQ ID NO:459, and/or (ii) a HA1 Globular Head Region aa sequence having at least 96% sequence identity with SEQ ID NO: 552.

48. The vaccine of clause 47, wherein: the virus strain from Influenza B-CVG-1 is Vic_ML04 having HA SEQ ID NO:553, and/or is Vic_NV11 having HA SEQ ID NO:554; and/or the virus strain from Influenza B-CVG-2 is Yam_TX11 having HA SEQ ID NO:555, and/or is Yam_PH13 having HA SEQ ID NO:556.

49. The vaccine of clause 48, wherein: the virus strain from Influenza B-CVG-1 is Vic_ML04 having HA SEQ ID NO:553, and/or is Vic_NV11 having HA SEQ ID NO:554; and the virus strain from Influenza B-CVG-2 is Yam_TX11 having HA SEQ ID NO:555, and/or is Yam_PH13 having HA SEQ ID NO:556.

50. The vaccine of clause 48, wherein: the virus strain from Influenza B-CVG-1 is Vic_ML04; and/or the virus strain from Influenza B-CVG-2 is Yam_TX11.

51. The vaccine of clause 50, wherein: the virus strain from Influenza B-CVG-1 is Vic_ML04; and the virus strain from Influenza B-CVG-2 is Yam_TX11.

52. The vaccine of any one of clauses 47-51, wherein: the Globular Head Region of the virus strain from Influenza B-CVG-1 comprises one or more predicted and/or confirmed N-linked glycosylation site(s) (NLGs); and/or the Globular Head Region of the virus strain from Influenza B-CVG-2 comprises one or more predicted and/or confirmed NLGs.

53. The vaccine of clause 52, wherein: the Globular Head Region of the virus strain from Influenza B-CVG-1 comprises one or more predicted and/or confirmed NLGs; and the Globular Head Region of the virus strain from Influenza B-CVG-2 comprises one or more predicted and/or confirmed NLGs.

54. The vaccine of clause 52, wherein: the Globular Head Region of the virus strain from Influenza B-CVG-1 comprises at least five predicted and/or confirmed NLGs; and/or the Globular Head Region of the virus strain from Influenza B-CVG-2 comprises at least five predicted and/or confirmed NLGs.

55. The vaccine of clause 54, wherein: the Globular Head Region of the virus strain from Influenza B-CVG-1 comprises at least five predicted and/or confirmed NLGs; and the Globular Head Region of the virus strain from Influenza B-CVG-2 comprises at least five predicted and/or confirmed NLGs.

56. The vaccine of any one of clauses 47-55, wherein the HA protein or HA1-containing portion thereof from each of the two component virus groups are present as one or more components that can be administered together, or sequentially.

57. The vaccine of clause 56, wherein the HA protein or HA1-containing portion thereof from each of the two component virus groups are combined in a multivalent vaccine composition for coadministration.

58. The vaccine of any one of clauses 47-57, further comprising an adjuvant, and/or a pharmaceutically acceptable carrier, diluent, or excipient.

59. The vaccine of clause 58, wherein the adjuvant comprises one or more aluminum salts.

60. The vaccine of any one of clauses 47-59, wherein, independently with respect to each of the two component virus groups, the HA protein(s) or the HA1-containing portion(s) thereof is present as one or more of: a component of an inactivated virus or component thereof; a component of a recombinant virus or component thereof; a recombinant HA or component thereof; and/or a component of a nanoparticle vaccine delivery platform/composition (e.g., including liposomes).

61. The vaccine of clause 60, wherein, independently with respect to each of the two component virus groups, the HA protein(s) or the HA1-containing portion(s) thereof is present as one or more of: a component of an inactivated virus or component thereof; and/or as a recombinant HA or component thereof.

62. The vaccine of clause 61, wherein, with respect to both component virus groups, the HA protein(s) or the HA1-containing portion(s) thereof is present as a component of an inactivated virus or component thereof, or as a recombinant HA or component thereof.

63. The vaccine of clause 62, wherein, with respect to both component virus groups, the HA protein(s) or the HA1-containing portion(s) thereof is present as a component of an inactivated virus or component thereof.

64. The vaccine of any one of clauses 47-63 or 72, wherein for each of the component virus groups, the vaccine comprises the HA protein(s), or the HA1-containing portion(s) thereof, of only one viral strain for each group.

65. A method of eliciting an immune response, comprising administering an immunogenic vaccine composition according to any one of claims 47-64 to a subject, thereby eliciting in the subject an immune response against influenza.

66. The method of clause 65, wherein eliciting the immune response comprises eliciting an Influenza B virus-specific immune response, and/or a pan-Influenza B virus-specific immune response.

67. The method of clause 66, wherein eliciting the immune response additionally comprises eliciting an immune response to at least one non-Influenza B vaccine strain.

68. The method of any one of clauses 65-67, wherein the immune response comprises one or more of an antibody, a B cell, and/or a T cell response.

69. The method of any one of clauses 65-68, wherein administration comprises administering the vaccine in one or more components administered together, or sequentially.

70. The method of any one of clauses 14-17, wherein inactivation comprises use of at least inactivating agent selected from HydroVax, formaldehyde, B-propiolactone (BPL), and binary ethylenimine (BEI).

71. The method of any one of clauses 37-40, wherein inactivation comprises use of at least inactivating agent selected from HydroVax, formaldehyde, B-propiolactone (BPL), and binary ethylenimine (BEI).

72. The method of any one of clauses 60-63, wherein inactivation comprises use of at least inactivating agent selected from HydroVax, formaldehyde, B-propiolactone (BPL), and binary ethylenimine (BEI).

73. A method of making a multivalent pan-influenza vaccine, comprising obtaining a viral haemagglutinin (HA) protein, or HA1-containing portion thereof, of or corresponding to a virus strain from each of any three of, or from all four of component virus strain groups H1-CVG1-H1-CVG-4 of clause 1, and combining or assembling the HA1 or the HA1-containg portions as one or more component parts of a multivalent pan-influenza vaccine.

74. The method of clause 73, wherein independently with respect to each of the three or the four component virus groups, the HA protein(s) or the HA1-containing portion(s) thereof is present as one or more of: a component of an inactivated virus or component thereof; a component of a recombinant virus or component thereof; a recombinant HA or component thereof; and/or a component of a nanoparticle vaccine delivery platform/composition (e.g., including liposomes).

75. A method of making a multivalent pan-influenza vaccine, comprising obtaining a viral haemagglutinin (HA) protein, or HA1-containing portion thereof, of or corresponding to a virus strain from each of any three of, or from all four of component virus strain groups H3-CVG-1-H3-CVG-4 of clause 24, and combining or assembling the HA1 or the HA1-containg portions as one or more component parts of a multivalent pan-influenza vaccine.

76. The method of clause 75, wherein independently with respect to each of the any three of, or the four component virus groups, the HA protein(s) or the HA1-containing portion(s) thereof is present as one or more of: a component of an inactivated virus component thereof; a component of a recombinant virus or component thereof; a recombinant HA or component thereof; and/or a component of a nanoparticle vaccine delivery platform/composition (e.g., including liposomes).

77. A method of making a multivalent pan-influenza vaccine, comprising obtaining a viral haemagglutinin (HA) protein, or HA1-containing portion thereof, of or corresponding to a virus strain from each of two component virus strain groups Influenza B-CVG-1 and Influenza B-CVG-2 of clause 47, and combining or assembling the HA1 or the HA1-containg portions as one or more component parts of a multivalent pan-influenza vaccine.

78. The method of clause 77, wherein independently with respect to each of the two component virus groups, the HA protein(s) or the HA1-containing portion(s) thereof is present as one or more of: a component of an inactivated virus or component thereof; a component of a recombinant virus or component thereof; a recombinant HA or component thereof; and/or a component of a nanoparticle vaccine delivery platform/composition (e.g., including liposomes).

### BRIEF DESCRIPTION OF THE DRAWINGS

FIG. 1 shows, by way of non-limiting examples of the present invention, a phylogenetic analysis of influenza A H1N1 sequences based on H1N1 all antigenic sites combined (H1-AASC) sequence as described herein.

FIG. 2 shows, by way of non-limiting examples of the present invention, a phylogenetic analysis of influenza A H1N1 sequences based on the H1N1 HA1 globular head (H1-HA1 Globular Head) sequence as described herein.

FIG. 3 shows, by way of non-limiting examples of the present invention, a phylogenetic analysis of Influenza A H3N2 sequences based on the H3N2 all antigenic sites combined (H3-AACS) sequence as described herein.

FIG. 4 shows, by way of non-limiting examples of the present invention, a phylogenetic analysis of Influenza A H3N2 sequences based on the H3N2 HA1 globular head (H3-HA1 Globular Head) sequence as described herein.

FIG. 5 shows, by way of non-limiting examples of the present invention, a phylogenetic analysis of Influenza B sequences based on the Influenza B-all antigenic sites combined (Influenza B-AASC) sequence as described herein.

FIG. 6 shows, by way of non-limiting examples of the present invention, a phylogenetic analysis of Influenza B sequences based on the Influenza B-HA1 Globular Head sequence as described herein.

FIGS. 7A and 7B show, by way of non-limiting examples of the present invention, that Multivalent HydroVax-H1N1 Influenza formulations provide broad immunity against homologous and heterologous influenza strains in mice.

FIGS. 8A and 8B show, by way of non-limiting examples of the present invention, that multivalent HydroVax-H1N1 Influenza formulations provide broad immunity against homologous and heterologous influenza strains in rhesus macaques.

FIGS. 9A and 9B show, by way of non-limiting examples of the present invention, that component virus group (CVG) members may be used interchangeably and still achieve broad homologous and heterologous immunity.

FIGS. 10A and 10B show, by way of non-limiting examples of the present invention, that HydroVax-Influenza provides protection of mice against homologous and heterologous live virus challenge.

FIGS. 11A and 11B show, by way of non-limiting examples of the present invention, that Multivalent HydroVax-H1N1 Influenza formulations provide broad immunity against homologous and heterologous influenza strains in ferrets.

FIG. 12 shows, by way of non-limiting examples of the present invention, that Multivalent HydroVax-H1N1 Influenza formulations provide protection against heterologous live virus challenge in ferrets.

### DETAILED DESCRIPTION OF THE INVENTION

Particular aspects of the present invention provide highly immunogenic, multivalent pan-influenza virus A and B vaccines (e.g., divalent, trivalent, tetravalent, etc.), methods for making same, and methods for eliciting an immune response against influenza by administering the vaccines to a subject.

Provided are Influenza A H1N1 multivalent vaccines comprising a viral haemagglutinin (HA) protein, or HA1-containing portion thereof, of/corresponding to at least one virus strain from each of any three of, or from each of all four H1N1 phylogenetically-derived component virus groups (H1-CVG-1-H1-CVG-4) as defined and claimed herein. The H1 HA1-Globular Head Region of the virus strain, independently from each of H1-CVG-1-H1-CVG4, may comprise one or more predicted and/or confirmed N-linked glycosylation site(s) (NLGs). Independently with respect to each of the three, or each of the four component virus groups (H1-CVG-1-H1-CVG-4), the HA protein or the HA1-containing portion thereof may, for example, be present as one or more of: a component of an inactivated virus or component thereof; a component of a recombinant virus or component thereof; a recombinant HA or component thereof; and/or a component of a nanoparticle vaccine delivery platform/composition (e.g., including liposomes). Preferably, with respect to the any three of, or the four component virus groups (H1-CVG-1-H1-CVG-4), the vaccine comprises the HA protein, or the HA1-containing portion thereof, of/corresponding to only one viral strain per group, and the HA protein or the HA1-containing portion thereof is present as a component of an inactivated virus or component thereof. The H1N1 multivalent vaccines may comprise the HA protein, or the HA1-containing portion thereof of/corresponding to more than one virus strain from each of the any three, or the four H1 component virus groups (H1-CVG-1-H1-CVG-4). With respect to trivalent vaccines comprising only three of the four component virus groups (H1-CVG-1-H1-CVG-4), the trivalent H1N1 multivalent vaccine preferably comprises the HA protein, or the HA1-containing portion thereof of/corresponding to at least one virus strain from H1-CVG-2 (preferably, or/corresponding to A/Fort Monmouth/1/1947 (FM47) or A/USSR/90/1977 (USSR77)).

Additionally provided are Influenza A H3N2 multivalent vaccines comprising a viral haemagglutinin (HA) protein, or HA1-containing portion thereof, of/corresponding to at least one virus strain from each of any three of, or from each of all four H3N2 phylogenetically-derived component virus groups (H3-CVG-1-H3-CVG-4) as defined and claimed herein. The H3 HA1-Globular Head Region of the virus strain, independently from H3-CVG-1-H3-CVG4, may comprise one or more predicted and/or confirmed N-linked glycosylation site(s) (NLGs). Independently with respect to each of the three, or each of the four component virus groups (H3-CVG-1-H3-CVG-4), the HA protein or the HA1-containing portion thereof may, for example, be present as one or more of: a component of an inactivated virus or component thereof; a component of a recombinant virus or component thereof; a recombinant HA or component thereof; and/or a component of a nanoparticle vaccine delivery platform/composition (e.g., including liposomes). Preferably, with respect to the any three of, or the four component virus groups (H3-CVG-1-H3-CVG-4), the vaccine comprises the HA protein, or the HA1-containing portion thereof, of/corresponding to only one viral strain per group, and the HA protein or the HA1-containing portion thereof is present as a component of an inactivated virus or component thereof. The H3N2 multivalent vaccines may comprise the HA protein, or the HA1-containing portion thereof of more than one virus strain from each of the any three, or the four H3 component virus groups (H3-CVG-1-H3-CVG-4).

Further provided are Influenza B multivalent vaccines comprising a viral haemagglutinin (HA) protein, or HA1-containing portion thereof, of/corresponding to at least one virus strain from each of two Influenza B phylogenetically-derived component virus groups (Influenza B-CVG-1 and Influenza B-CVG-2) as defined and claimed herein. The Influenza B-Globular Head Region of the virus strain, independently from Influenza B-CVG-1 and Influenza B-CVG-2, may comprise one or more predicted and/or confirmed N-linked glycosylation site(s) (NLGs). Independently with respect to the two component virus groups (Influenza B-CVG-1 and Influenza B-CVG-2), the HA protein or the HA1-containing portion thereof may be present, for example, as one or more of: a component of an inactivated virus or component thereof; a component of a recombinant virus or component; a recombinant HA or component thereof; and/or a component of a nanoparticle vaccine delivery platform/composition (e.g., including liposomes). Preferably, with respect to the two component virus groups (Influenza B-CVG-1 and Influenza B-CVG-2), the vaccine comprises the HA protein, or the HA1-containing portion thereof, of/corresponding to only one viral strain per group, and the HA protein or the HA1-containing portion thereof is present as a component of an inactivated virus or component thereof. The Influenza B multivalent vaccines may comprise the HA protein, or the HA1-containing portion thereof of more than one virus strain from each of the two Influenza B component virus groups (Influenza B-CVG-1 and Influenza B-CVG-2).

### Influenza Hemagglutinin (HA) Protein

The hemagglutinin (HA) protein is the dominant surface glycoprotein found on influenza virus particles, and immunity against HA is widely recognized as key to protection against disease. HA monomers assemble into trimers on the virus surface and are initially expressed as intact protein, termed HA0. During virus maturation each HA0 monomer is cleaved by host cellular proteases into HA1 and HA2 subunits, which remain attached through disulfide linkages. The HA2 subunit plays a largely structural role, providing a stem/stalk architecture that supports the surface-exposed globular HA1 subunit, and also anchors the entire HA protein to the virus envelope through a C-terminal transmembrane domain. Conversely, the globular head of the surface exposed HA1 subunit binds to monosaccharide sialic acids present on the surface of target cells. As such, this subunit is the primary target of the host immune response, and the virus may incorporate direct changes in the amino acid sequence, and/or the addition of N-linked glycosylations, to evade this immune response.

Given its central role in anti-influenza immunity, a significant amount of research has been directed at understanding regions of the HA1 subunit involved in immune evasion. Gerhard et al. originally described four ‘operationally distinct’ antigenic sites based on the binding of monoclonal antibodies to a range of mutated H1N1 viruses, termed; Sa, Sb, Ca and Cb (Gerhard, W., et al, Antigenic structure of influenza virus haemagglutinin defined by hybridoma antibodies. Nature, 1981. 290 (5808): p. 713-7). A subsequent study by this same group expanded this list of antigenic sites to Sa, Sb, Ca₁, Ca₂ and Cb (Caton, A. J., et al, The antigenic structure of the influenza virus A/PR/8/34 hemagglutinin (H1 subtype). Cell, 1982. 31 (2 Pt 1): p. 417-27). Subsequent studies (Brownlee & Fodor, The predicted antigenicity of the haemagglutinin of the 1918 Spanish influenza pandemic suggests an avian origin. Philos Trans R Soc Lond B Biol Sci, 2001. 356 (1416): p. 1871-6. PMCID: PMC1088563) defined the exact amino acid composition of these antigenic sites.

### Immunogenicity Studies

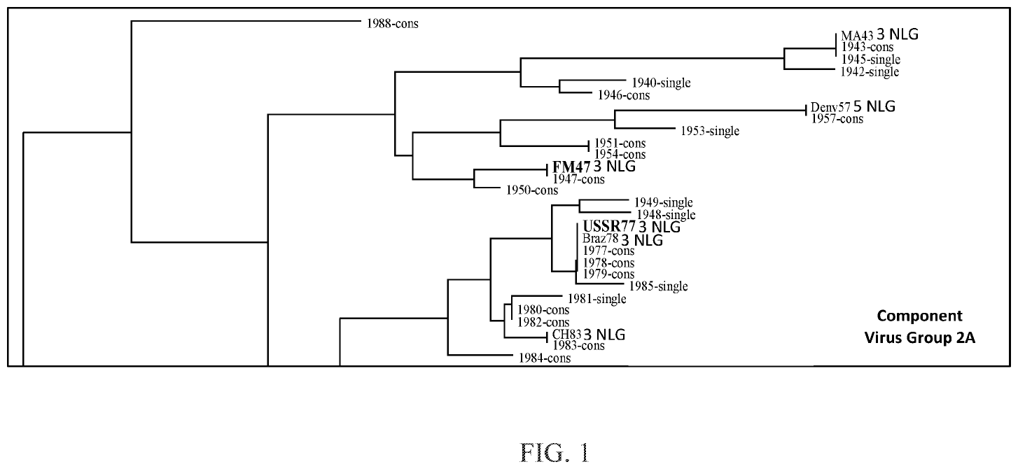

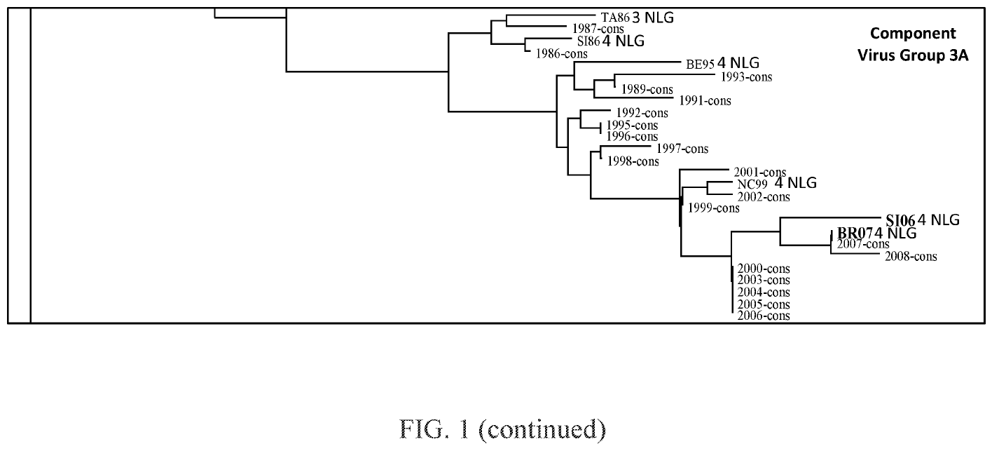

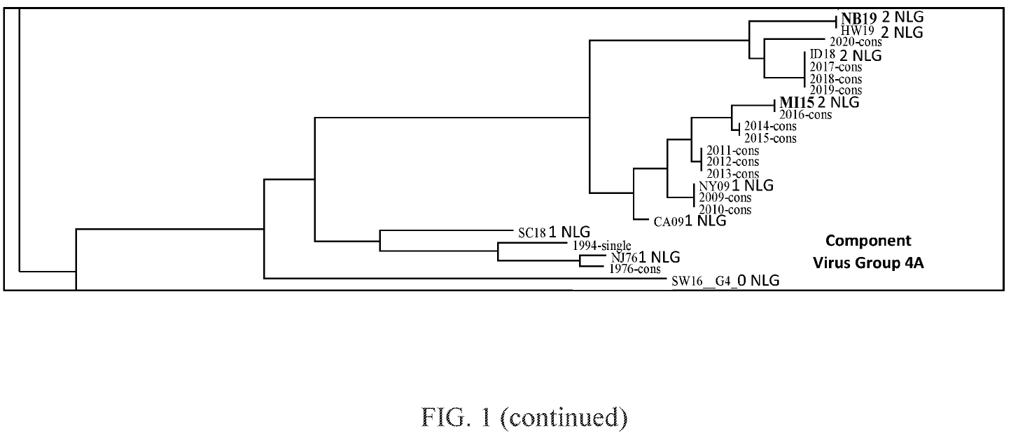

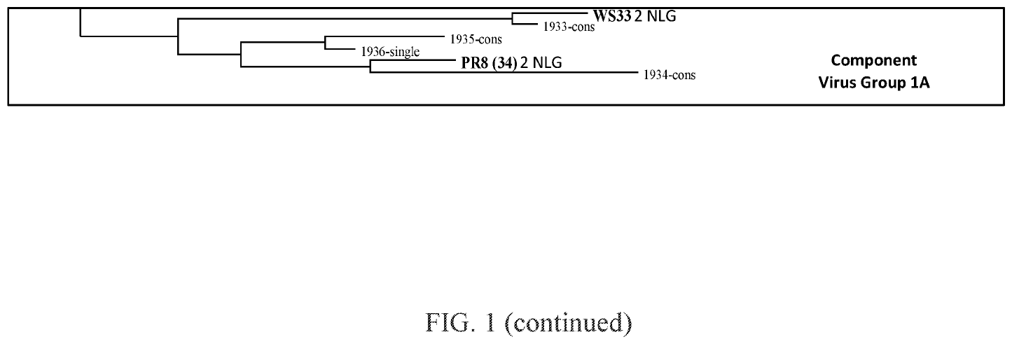

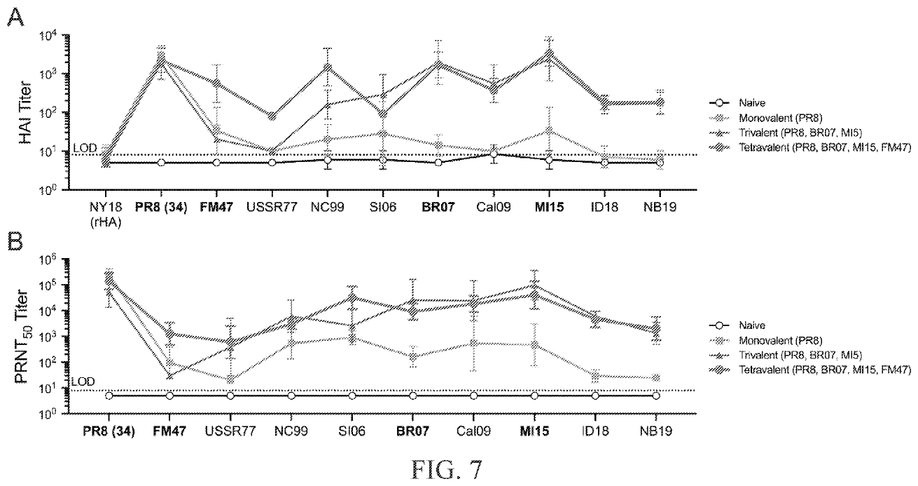

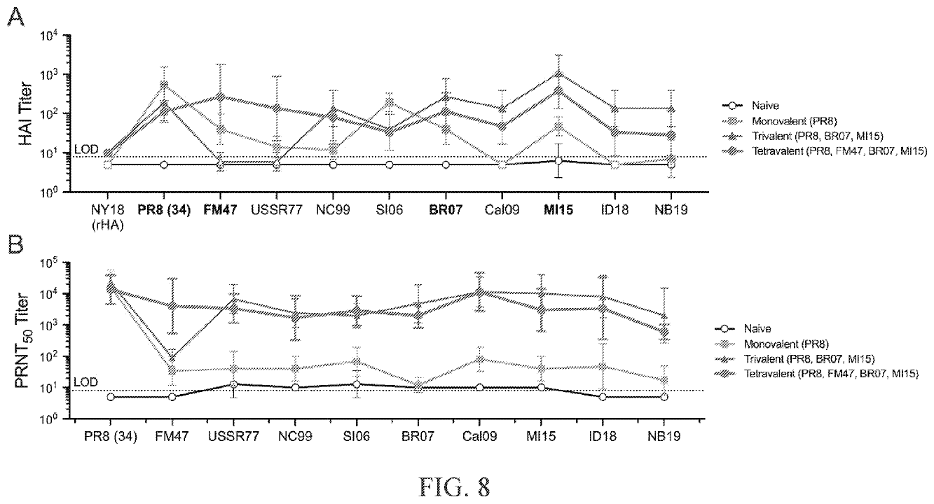

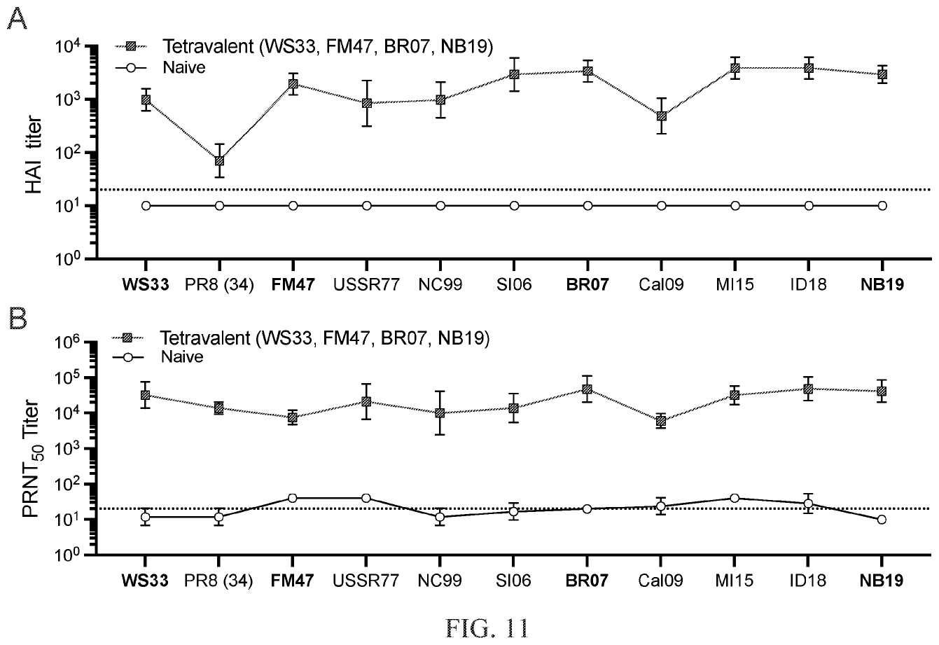

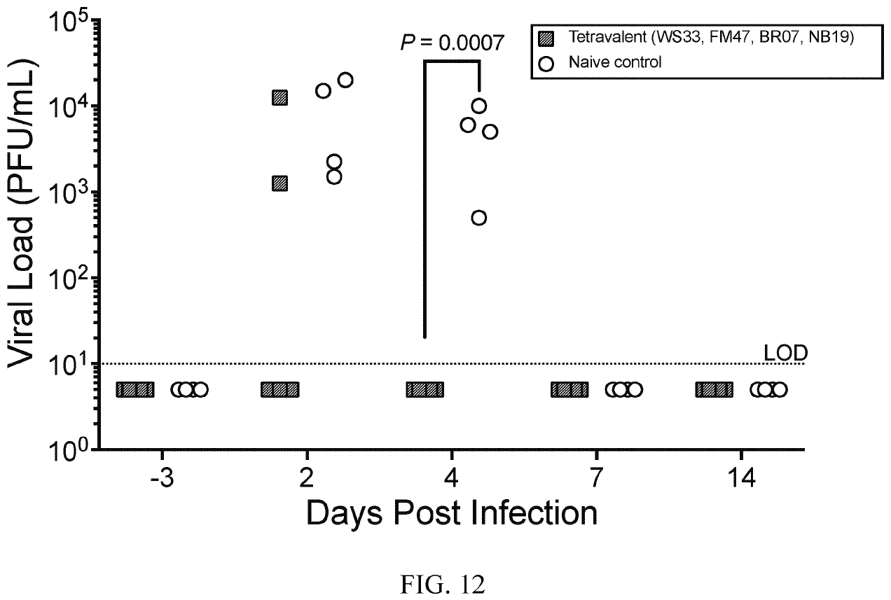

Influenza A H1N1. Using phylogenetic analyses described in detail under working Examples 2, 3 and 4 below, and as shown in FIGS. 1-6, Influenza A and B vaccine formulations (e.g., whole virus inactivated vaccine formulations) of varying valencies were developed. According to aspects of the present invention, Influenza A and B multivalent vaccine formulations provide improved breadth relative to monovalent formulations, and a multivalent strategy was developed based on identification and selection of strains from distinct Component Virus Groups (CVG) defined by the disclosed phylogenetic studies (see Examples 2, 3 and 4 below). For example, exemplary Influenza A H1N1 vaccine formulations included the following: monovalent—PR8 (34) from H1-CVG1A/1B; trivalent—PR8 (34) from H1-CVG1A/1B, BR07 from H1-CVG3A/3B and MI15 from H1-CVG4A/4B; tetravalent—PR8 (34) from H1-CVG1A/1B, FM47 from H1-CVG2A/2B, BR07 from H1-CVG3A/3B and MI15 from H1-CVG4A/4B. These exemplary H1N1 vaccine formulations were tested in mouse (FIGS. 7A, 7B, 9A, 9B, 10A and 10B), non-human primate (FIGS. 8A and 8B), and ferret (FIGS. 11A, 11B and 12) animal models, with immunity assessed by both hemagglutination inhibition (HAI) assay (HAI) and plaque reduction neutralization-50% (PRNT₅₀) assay (PRNT₅₀), as well as heterologous live virus challenge.

For the data shown in FIG. 7, BALB/c mice (n=4-5 per group) were immunized intraperitoneally with a monovalent, trivalent or tetravalent formulation of HydroVax™—H1N1 on days 0 and 28. Formulations were comprised of the indicated combinations of the following virus strains: A/Puerto Rico/8/1934 [PR8 (34)], A/Fort Monmouth/1/1947 (FM47), A/Brisbane/59/2007 (BR07) and A/Michigan/45/2015 (MI15). Each strain was individually purified and HydroVax-inactivated prior to blending (trivalent and tetravalent formulations only) followed by adsorption to 0.20% aluminum hydroxide. Vaccine doses contained 1 mcg of each virus component. Serum samples were collected at day 42 (14 days after the final vaccination) and assessed for immunogenicity by either the hemagglutination inhibition assay (HAI) (FIG. 7A) or the 50% plaque reduction neutralization test (PRNT₅₀) (FIG. 7B). Immunogenicity tests were performed across a broad chronological range of H1N1 strains, including the homologous vaccine viruses (indicated in bold) as well as non-homologous viruses such as A/USSR/90/1977 (USSR77), A/New Caledonia/20/1999 (NC99), A/Solomon Islands/3/2006 (SI06), A/California/07/2009 (Cal09), A/Idaho/07/2018 (ID18) and A/Nebraska/14/2019 (NB19). Due to biosafety concerns, the A/New York/1/1918 (NY18) pandemic strain is only available as a recombinant hemagglutinin protein (rHA), therefore, the PRNT₅₀ assay could not be performed with this strain. Group geometric mean titers (GMT) are shown with their associated 95% confidence intervals. The limit of detection (LOD) for each assay is indicated by the dotted lines.

In the mouse model, as shown in FIGS. 7A and 7B, the monovalent PR8-based vaccine provided robust homologous immunity by both HAI and PRNT₅₀, but responses against non-homologous virus strains were more than 10- to 100-fold reduced. The trivalent vaccine, with components from the H1-CVG1A/1B, H1-CVG3A/3B and H1-CVG4A/4B groupings provided a more balanced immune response, even to non-homologous virus strains including NC99, SI06, ID18 and NB19. However, a clear gap in breadth was still observed with strains FM47 and USSR77, which are both members of the H1-CVG2A/2B grouping. This gap was resolved through the use of a tetravalent vaccine that contained FM47 for the H1-CVG2A/2B group, resulting in enhanced immunity not only to the homologous FM47 virus, but also to the non-homologous USSR77 strain.

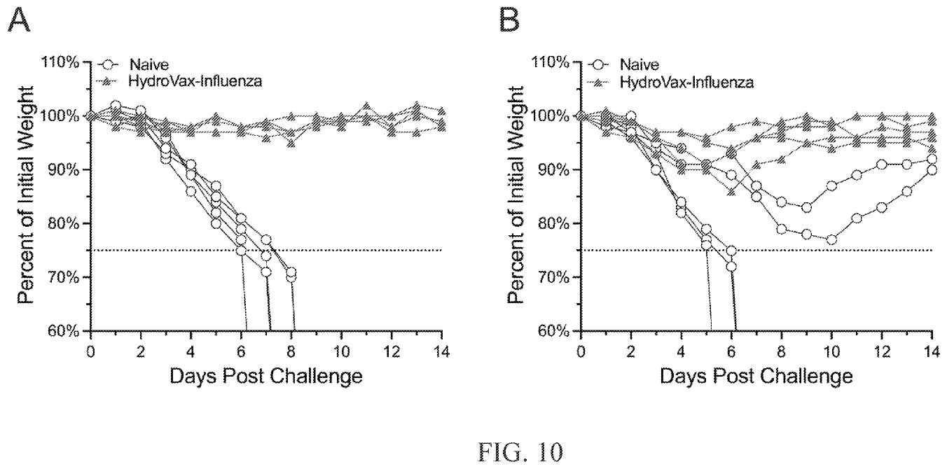

As an additional measure of the breadth of immunity, homologous and non-homologous challenge studies were performed following vaccination of mice with a multivalent formulation (FIG. 10). In brief, mice were immunized with the trivalent H1N1 combination (PR8 [34], BR07, and MI15) and challenged with a lethal dose of live PR8 (FIG. 10A) or Cal09 (FIG. 10B), a non-homologous H1-CVG4A/4B virus strain. In more detail, BALB/c mice were immunized intraperitoneally with a multivalent H1N1 HydroVax-Influenza vaccine, which included 1 mcg each of the following inactivated influenza virus strains, A/Puerto Rico/8/1934 [PR8 (34)], A/Brisbane/59/2007 (BR07) and A/Michigan/45/2015 (MI15), adsorbed to 0.20% aluminum hydroxide adjuvant. Vaccines were administered on days 0 and 28, with mice challenged on day 170. Mice were challenged intranasally with either (FIG. 10A) a vaccine-homologous strain of virus (PR8 (34); 800 PFU=estimated 20 LD₅₀) or (FIG. 10B) a non-homologous strain of influenza (A/California/07/2009, 1262 PFU=estimated 20 LD₅₀). Animals were monitored daily for weight and humanely euthanized if values fell below 75% of their initial starting weight, as indicated by the dotted line. In both instances, the vaccine provided complete protection from lethal disease, compared to 60-100% lethality in unvaccinated mice.

Similar vaccination studies were carried out in rhesus macaques to assess the robustness of the disclosed approach across multiple animal species (FIGS. 8A and 8B). For the data of FIGS. 8A and 8B, rhesus macaques (n=4 per group) were immunized intramuscularly with a monovalent formulation of the HydroVax-H1N1 vaccine on days 0, 28 and 180 or indicated multivalent formulations on days 0, 28 and 120. Formulations were comprised of the indicated combinations of the following virus strains: A/Puerto Rico/8/1934 [PR8 (34)], A/Fort Monmouth/1/1947 (FM47), A/Brisbane/59/2007 (BR07) and A/Michigan/45/2015 (MI15). Each strain was individually purified and inactivated prior to blending (trivalent and tetravalent formulations only) followed by adsorption to 0.20% aluminum hydroxide. Vaccine doses contained 15 mcg of PR8 in the monovalent formulation or 10 mcg of each virus component in the multivalent formulations. Serum samples were collected at 14 days after the final vaccination and assessed for immunogenicity by either the (FIG. 8A) hemagglutination inhibition assay (HAI) or the (FIGS. 8B) 50% plaque reduction neutralization test (PRNT₅₀). Immunogenicity tests were performed across a broad chronological range of H1N1 strains, including the homologous vaccine viruses (indicated in bold) as well as non-homologous viruses such as A/USSR/90/1977 (USSR77), A/New Caledonia/20/1999 (NC99), A/Solomon Islands/3/2006 (SI06), A/California/07/2009 (Cal09), A/Idaho/07/2018 (ID18) and A/Nebraska/14/2019 (NB19). Due to biosafety concerns, the A/New York/1/1918 (NY18) pandemic strain is only available as a recombinant hemagglutinin protein (rHA), therefore, the PRNT₅₀ assay could not be performed with this strain. Group geometric mean titers (GMT) are shown with their associated 95% confidence intervals. The limit of detection (LOD) for each assay is indicated by the dotted lines. As with the mouse studies, the PR8-only monovalent vaccine provided strong immunity against the homologous virus strain, but limited immunity against other virus strains. Breadth increased dramatically with the trivalent vaccine and reached balanced immunity against all tested virus strains when using the tetravalent vaccine, which included representative vaccine strains from all component virus groups.

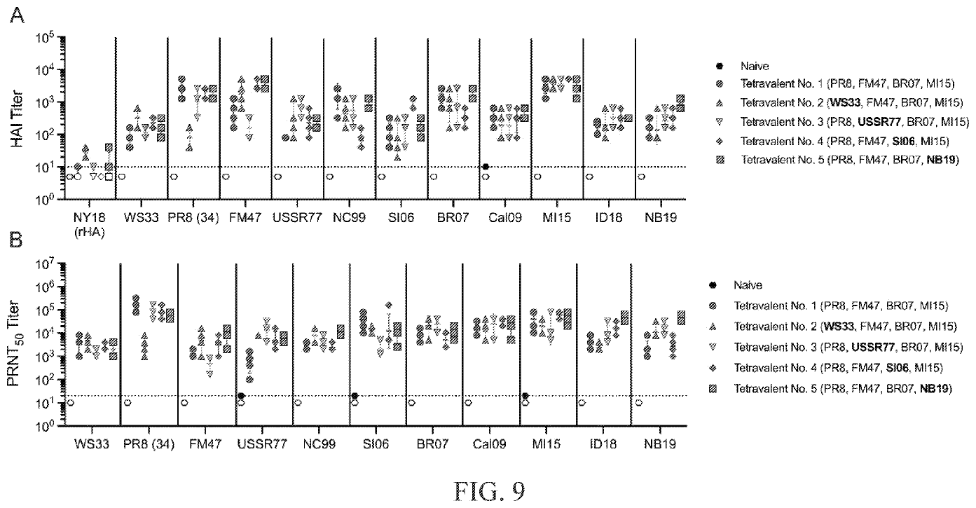

To yet further characterize the disclosed multivalent influenza vaccine development approach, an examination was made as to whether vaccine components could be replaced with alternative virus strains from the same grouping and still maintain robust breadth of immunity (FIGS. 9A and 9B). These studies used the prior tetravalent combination (PR8[34], FM47, BR07 and MI15) as a reference approach, with each strain representing a member of the defined phylogenetic groupings, H1-CVG1A/1B through CVG4A/4B. We then prepared additional tetravalent formulations wherein a single component was replaced with a virus strain from the same component virus group. These included the following

combinations: WS33, FM47, BR07, MI15; PR8[34], USSR77, BR07, MI15; PR8[34], FM47, SI06, MI15; and PR8[34], FM47, BR07, NB19. In more detail, for the data of FIGS. 9A and 9B, BALB/c mice (n=4-5 per group) were immunized intraperitoneally with tetravalent formulations of HydroVax-H1N1 on days 0 and 28. Formulations were comprised of the indicated combinations of the following virus strains: H1N1 Component Virus Group 1A/B=A/Puerto Rico/8/1934 [PR8 (34)] or A/WSN/1933 (WS33); H1N1 Component Virus Group 2A/B=A/Fort Monmouth/1/1947 (FM47) or A/USSR/90/1977 (USSR77); H1N1 Component Virus Group 3A/B=A/Brisbane/59/2007 (BR07) or A/Solomon Islands/3/2006 (SI06); H1N1 Component Virus Group 4A/B=A/Michigan/45/2015 (MI15) or A/Nebraska/14/2019 (NB19). Each strain was individually purified and inactivated prior to blending followed by adsorption to 0.20% aluminum hydroxide. Vaccine doses contained 1 mcg of each virus component. Serum samples were collected at day 42 (14 days after the final vaccination) and assessed for immunogenicity by either the (A) hemagglutination inhibition assay (HAI) or the (B) 50% plaque reduction neutralization test (PRNT₅₀). Immunogenicity tests were performed across a broad chronological range of H1N1 strains, including the homologous vaccine viruses as well as non-homologous viruses including A/New Caledonia/20/1999 (NC99), A/California/07/2009 (Cal09), and A/Idaho/07/2018 (ID18). Due to biosafety concerns, the A/New York/1/1918 (NY18) pandemic strain is only available as a recombinant hemagglutinin protein (rHA), therefore, the PRNT₅₀ assay could not be performed with this strain. Individual sample results are shown as well as group geometric mean titers (GMT) and their associated 95% confidence intervals. The limit of detection (LOD) for each assay is indicated by the dotted lines. Samples below the LOD are indicated as open symbols.

Similar multivalent H1N1 vaccine formulation vaccination studies were carried out in ferrets to yet further assess the robustness of the disclosed approach across multiple animal species (FIGS. 11A, 11B and 12). For the data of FIGS. 11A and 11B, ferrets (n=4-5 per group) were immunized intramuscularly with a multivalent formulation of the HydroVax-H1N1 vaccine on days 0, 28 and 90, or left untreated as naïve controls. The formulation was comprised of the following virus strains: A/WSN/1933 (WS33), A/Fort Monmouth/1/1947 (FM47), A/Brisbane/59/2007 (BR07) and A/Nebraska/14/2019 (NB19). Each strain was individually purified and inactivated by the HydroVax approach prior to blending followed by adsorption to 0.20% aluminum hydroxide. Vaccine doses contained 10 mcg of each virus component. Serum samples were collected at 15 days after the final vaccination and assessed for immunogenicity by either the hemagglutination inhibition assay (HAI) (FIG. 11A) or the 50% plaque reduction neutralization test (PRNT₅₀) (FIG. 11B). Immunogenicity tests were performed across a broad chronological range of H1N1 strains, including the homologous vaccine viruses (indicated in bold) as well as non-homologous viruses such as A/Puerto Rico/8/1934 [PR8 (34)], A/USSR/90/1977 (USSR77), A/New Caledonia/20/1999 (NC99), A/Solomon Islands/3/2006 (SI06), A/California/07/2009 (Cal09), A/Michigan/45/2015 (MI15), and A/Idaho/07/2018 (ID18). Group geometric mean titers (GMT) are shown with their associated 95% confidence intervals. The limit of detection (LOD) for each assay is indicated by the dotted lines. As with the mouse and rhesus macaque studies, broad and balanced immunity was observed against all tested virus strains when using the tetravalent vaccine, which included representative vaccine strains from all HIN1 component virus groups. As an additional measure of the breadth of immunity, a non-homologous challenge study was performed in these same animals (FIG. 12). At study day 180, ferrets were challenged intranasally with a non-homologous strain of influenza (A/California/07/2009, 1E7 PFU per animal). Animals were monitored for nasal viral shedding 3 days prior to challenge (day −3) and on days 2, 4, 7 and 14 post-infection. The HydroVax-H1N1 vaccine demonstrated a decrease in the number of viremic animals at day 2 post-infection compared to naïve animals and provided full protection against virus shedding at day 4 post-infection (100% virus positive in naïve animals, 0% in vaccinated animals, P=0.0007 by ANOVA with multiple test correction).

In all species tested, and despite employing distinct virus strains, robust immunity to both homologous and non-homologous virus strains was still observed, indicating that the disclosed phylogenetically-defined component virus groups (CVGs) provide a robust approach to selecting appropriate influenza virus strains for effective pan-H1N1 multivalent vaccine formulations.

Using the disclosed methods, immunogenic compositions, such as multivalent Influenza A and B vaccines containing, for example, inactivated virus strains are also provided. For example, the composition (or medicament) can be a lyophilized immunogenic composition (e.g., vaccine preparation) containing viral antigens that retain one or more predominant antigenic epitopes of the biologically active pathogen from which it was prepared, or from which it corresponds. The lyophilized composition may be prepared preservative-free and devoid of any inactivating agent (e.g., devoid of H₂O₂, etc.). The composition can also be a liquid prepared by reconstituting a lyophilized composition in a pharmaceutically acceptable diluent. Optionally, the composition can include a suitable adjuvant that increases the antigenic efficacy of the antigen.

### Methods for Eliciting an Immune Response in a Subject by Administering the Disclosed Vaccine Compositions are Also Provided

Methods of eliciting an immune response against a pathogen by administering the immunogenic compositions are provided. Typically, the immune response is a protective immune response that prevents or reduces infection by one or more pathogens. For example, an immune response can be elicited in a subject by administering the vaccine composition to a subject, thereby eliciting in the subject an immune response (e.g., a protective immune response) against the pathogen. In some applications the solution is administered to a subject using any method suitable for delivering a vaccine to a subject, e.g., intramuscular, intradermal, transdermal, subcutaneous or intravenous injection, oral delivery, or intranasal or other mucosal delivery of the immunogenic composition (e.g., vaccine).

### Terms

Unless otherwise explained, all technical and scientific terms used herein have the same meaning as commonly understood by one of ordinary skill in the art to which this disclosure belongs. Definitions of common terms in molecular biology may be found in Benjamin Lewin, Genes V, published by Oxford University Press, 1994 (ISBN 0-19-854287-9); Kendrew, et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

The singular terms “a,” “an,” and “the” include plural referents unless context clearly indicates otherwise. Similarly, the word “or” is intended to include “and” unless the context clearly indicates otherwise.

Although methods and materials similar or equivalent to those described herein can be used in the practice or testing of this disclosure, suitable methods and materials are described below. The term “comprises” means “includes.” The abbreviation, “e.g.” is derived from the Latin exempli gratis, and is used herein to indicate a non-limiting example. Thus, the abbreviation “e.g.” is synonymous with the term “for example.

In order to facilitate review of the various embodiments of this disclosure, the following explanations of specific terms are provided:

“An immunogenic composition” or “vaccine composition” or “vaccine” is a composition of matter suitable for administration to a human or animal subject that is capable of eliciting a specific immune response, e.g., against a pathogen. As such, an immunogenic composition or vaccine includes one or more antigens or antigenic epitopes. The antigen can be, for example, in the context of an isolated protein or peptide fragment of a protein, such as split-inactivated or recombinant protein vaccines, or can be a partially purified preparation derived from a pathogen. Alternatively, the antigen can be in the context of a whole live or inactivated pathogen. Typically, when an immunogenic composition or vaccine includes a live pathogen, the pathogen is attenuated, that is, incapable of causing disease in an immunologically competent subject. In other cases, an immunogenic composition or vaccine includes a whole inactivated (or killed) pathogen. The inactivated pathogen can be either a wild-type pathogenic organism that would otherwise (if not inactivated) cause disease in at least a portion of immunologically competent subjects, or an attenuated or mutant strain or isolate of the pathogen. In the context of this disclosure, the immunogenic and/or vaccine compositions preferably contain a whole (wild-type, attenuated or mutant) pathogen (e.g., Influenza virus A or B strains) that is either inactivated or incapable of causing disease in human or animal subject to which the vaccine composition is administered.

An “immune response” is a response of a cell of the immune system, such as a B cell, T cell, or monocyte, to a stimulus. In some cases, an immune response is a T cell response, such as a CD4+ response or a CD8+ response. Alternatively, the response is a B cell response, and results in the production of specific antibodies. In some cases, the response is specific for a particular antigen (that is, an “antigen-specific response”). If the antigen is derived from a pathogen, the antigen-specific response is a “pathogen-specific response.” A “protective immune response” is an immune response that inhibits a detrimental function or activity of a pathogen, reduces infection by a pathogen, or decreases symptoms (including death) that result from infection by the pathogen. A protective immune response can be measured, for example, by the inhibition of viral replication or plaque formation in a plaque reduction assay or ELISA-neutralization assay, or by measuring resistance to viral challenge in vivo.

An “immunologically effective amount” is a quantity of a composition used to elicit an immune response in a subject. In the context of a vaccine administration, the desired result is typically a protective pathogen-specific immune response. However, to obtain protective immunity against a pathogen in an immunocompetent subject, multiple administrations of the vaccine composition may be required. Thus, in the context of this disclosure, the term immunologically effective amount encompasses a fractional dose that contributes in combination with previous or subsequent administrations to attaining a protective immune response.

An “antigen” is a compound, composition, or substance that can stimulate the production of antibodies and/or a T cell response in an animal, including compositions that are injected, absorbed or otherwise introduced into an animal. The term “antigen” includes all related antigenic epitopes. The term “epitope” or “antigenic determinant” refers to a site on an antigen to which B and/or T cells respond.

The “predominant antigenic epitopes” are those epitopes to which a functionally significant host immune response, e.g., an antibody response or a T-cell response, is made. Thus, with respect to a protective immune response against a pathogen, the predominant antigenic epitopes are those antigenic moieties that when recognized by the host immune system result in protection from disease caused by the pathogen.

The term “antigenicity” refers to the relative maintenance of immunogenic epitope structure(s) as determined, for example, by various in vitro measurements, such as binding of specific monoclonal antibodies or hemagglutination assays. “Antigenicity” in the in vivo context is typically referred to herein as “immunogenicity.”

An “adjuvant” is an agent that enhances the production of an immune response in a non-specific manner. Common adjuvants include suspensions of minerals (e.g., alum, aluminum hydroxide, aluminum phosphate) onto which antigen is adsorbed; or water-in-oil emulsions in which an antigen solution is emulsified in oil (MF-59, Freund's incomplete adjuvant). Additional details regarding various adjuvants can be found in Derek O'Hagan Vaccine Adjuvants: Preparation Methods and Research Protocols (Methods in Molecular Medicine) Humana Press, 2000.

The term “pathogen” as used herein refers to an organism having either an RNA or DNA genome, and encompasses viruses (both RNA and DNA genome-based). In particular preferred aspects, “pathogen” refers to an Influenza virus A or B strains.

The term “whole pathogen” refers to a pathogenic organism, such as a virus, that includes all or substantially all of the constituents of the infectious form of the organism. Typically, a whole pathogen is capable of replication. The term “whole pathogen” is nonetheless distinct from the term “wild-type” pathogen, and the term “whole pathogen” encompasses wild-type as well as attenuated and other mutant forms of the pathogenic organism. Thus, a whole pathogen can be an attenuated pathogen incapable of causing disease in an immunocompetent host, but nonetheless including all or substantially all of the constituents of an infectious pathogen. Similarly, a whole pathogen can be a mutant form of the pathogen, lacking one or more intact (wild-type) genes, and/or proteins. The pathogen genome may comprise RNA or DNA.

An “inactivated pathogen” is a whole pathogen that has been rendered incapable of causing disease (e.g., rendered noninfectious) by artificial means. Typically, an inactivated pathogen is a “killed pathogen” that is incapable of replication. A pathogen is noninfectious when it is incapable of replicating or incapable of replicating to sufficient levels to cause disease.

An “immunogenically active vaccine,” as used herein in connection with Applicants' methods, is a pathogen inactivated by the disclosed methods that is capable of eliciting an immune response when introduced into an immunologically competent subject. The immune response produced in response to exposure to an immunogenically active vaccine comprising the inactivated pathogen as disclosed herein is preferably identical, substantially identical, or superior with respect to that produced by the predominant antigenic epitopes of the respective infectious pathogen.

The phrase “of or corresponding to” as used herein, refers to the nature/source of the haemagglutinin (HA) protein or HA1-containing portion thereof. For example, the HA protein or HA1-containing portion thereof may be “of” (i.e., taken directly from) a virus (wild-type, mutant, attenuated, etc.) or portion thereof, or split (detergent/chemical disrupted) portion thereof. Alternatively, the HA protein or HA1-containing portion thereof may “correspond to” the virus, being recombinantly derived (e.g., DNA or RNA based expression of the HA protein or the HA1-containing portion thereof, and/or use of vector-mediated expression of the HA protein or the HA1-containing portion thereof), or being a synthetic HA protein or HA1-containing portion thereof.

The verb “lyophilize” means to freeze-dry under vacuum. The process is termed “lyophilization.” In some cases, the sample to be dried (e.g., dehydrated) is frozen prior to drying. In other cases, the material to be dried is subjected to the drying process without prior phase change. During the process of lyophilization, evaporation of the solvent results in cooling of the sample to temperatures below the melting temperature of the solvent/solute mixture resulting in freezing of the sample. Solvent is removed from the frozen sample by sublimation. A product that has undergone lyophilization is “lyophilized.” As used in this disclosure the term lyophilization also encompasses functionally equivalent procedures that accelerate the drying process without exposing the sample to excessive heat, specifically including: spray drying and spray freeze-drying.

In the context of this disclosure “room temperature” refers to any temperature within a range of temperatures between about 16° C. (approximately 61° F.) and about 25° C. (approximately 77° F.). Commonly, room temperature is between about 20° C. and 22° C. (68° F.-72° F.). Generally, the term room temperature is used to indicate that no additional energy is expended cooling (e.g., refrigerating) or heating the sample or ambient temperature.

A “preservative” is an agent that is added to a composition to prevent decomposition due to chemical change or microbial action. In the context of vaccine production, a preservative is typically added to prevent microbial (e.g., bacterial and fungal) growth. The most common preservative used in vaccine production is thimerosal, a mercury containing organic compound. Thus, the term “preservative-free” indicates that no preservative is added to (or present in) the composition.

The term “purification” (e.g., with respect to a pathogen or a composition containing a pathogen) refers to the process of removing components from a composition, the presence of which is not desired. Purification is a relative term and does not require that all traces of the undesirable component be removed from the composition. In the context of vaccine production, purification includes such processes as centrifugation, dialization, ion-exchange chromatography, and size-exclusion chromatography, affinity-purification, precipitation and other methods disclosed herein (e.g., lyophilization, etc.). Such purification processes can be used to separate the inactivated pathogen components from the reagents used to inactivate the respective pathogen as disclosed herein. A range of standard purification techniques may be used to remove or separate these residual components from vaccine antigen prior to final formulation, including, but not limited to, affinity chromatography, ion-exchange chromatography, mixed-mode/multimodal chromatography, gel filtration/size-exclusion chromatography, desalting chromatography, tangential flow filtration/diafiltration, density-gradient centrifugation, centrifugal filtration, dialysis, vaccine antigen precipitation or vaccine antigen adsorption.

The adjective “pharmaceutically acceptable” indicates that the subject is physiologically acceptable for administration to a subject (e.g., a human or animal subject). Remington's Pharmaceutical Sciences, by E. W. Martin, Mack Publishing Co., Easton, PA, 15th Edition (1975), describes compositions and formulations (including diluents) suitable for pharmaceutical delivery of therapeutic and/or prophylactic compositions, including vaccines.

In general, the nature of the diluent will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. In certain formulations (for example, solid compositions, such as powder, pill, tablet, or capsule forms), a liquid diluent is not employed. In such formulations, non-toxic solid carriers can be used, including for example, pharmaceutical grades of mannitol, lactose, starch or magnesium stearate.

The phrase “Good Manufacturing Practice” or “GMP” with respect to methods and procedures employed in vaccine production refer specifically to the set of methods, protocols and procedures established by the United States Food and Drug Administration (FDA). Similar recommendations and guidelines are promulgated by the World Health Organization. The abbreviation “cGMP” specifically designates those protocols and procedures that are currently approved by the FDA (e.g., under 21 Code of Federal Regulations, parts 210 and 211, available on the world wide web at fda.gov/cder/dmpq). With time cGMP compliant procedures may change. Any methods disclosed herein can be adapted in accordance with new cGMP requirements as mandated by the FDA.

### Reconstitution and Administration

Immunogenic compositions, such as vaccines, that are produced as powders (e.g., lyophilized powders) are typically mixed with a liquid for administration. This process is known as “reconstitution,” and the liquid used is commonly referred to as a “diluent.” For purposes of administration, especially to human subjects, it is important that the diluent be a pharmaceutically acceptable formulation. Reconstitution of the lyophilized composition is typically carried out using a sterile syringe and needle for each vial of diluent. The correct diluent for each type and batch is used to ensure adequate potency, safety and sterility of the resulting mixture. Diluents are specifically designed to optimize delivery and efficacy of the selected composition. Common diluents include such additives as: stabilizers to improve heat stability of the vaccine; agents, such as surfactants, to assist in dissolving the powder into a liquid; and buffers to ensure the correct acidic balance of the reconstituted composition. Optionally, the diluent can contain a preservative (e.g., a bactericide and/or a fungicide) to maintain sterility after reconstitution. Preservatives are typically required (e.g., by the FDA) when the composition is reconstituted in a multi-dose formulation.

### Administration of Immunogenic Compositions Such as Vaccines (Therapeutic Methods)

The immunogenic compositions (such as vaccine or other medicaments) disclosed herein can be administered to a subject to elicit an immune response against a pathogen. Most commonly, the compositions are administered to elicit a prophylactic immune response against a pathogenic organism to which the subject has not yet been exposed. For example, vaccine compositions including dual oxidation-inactivated pathogens can be administered as part of a localized or wide-spread vaccination effort. An immune response elicited by administration of such vaccine compositions typically includes a neutralizing antibody response, and can in addition include a T cell response, e.g., a cytotoxic T cell response that targets cellular pathogens.

In some cases, the immunogenic composition can include a combination of pathogens, such as a combination of viruses (e.g., a combination of Influenza A H1N1 virus strains; a combination of Influenza A H3N2 virus strains; a combination of Influenza B virus strains, etc.).

The quantity of pathogen included in the composition is sufficient to elicit an immune response when administered to a subject. For example, when administered to a subject in one or more doses, a vaccine composition containing an inactivated pathogen favorably elicits a protective immune response against the pathogen. A dose of the vaccine composition can include at least about 0.1% wt/wt inactivated pathogen to about 99% wt/wt inactivated pathogen, with the balance of the vaccine composition is made up of pharmaceutically acceptable such as a pharmaceutically acceptable carrier and/or pharmaceutically acceptable diluent. Guidelines regarding vaccine formulation can be found, e.g., in U.S. Pat. Nos. 6,890,542, and 6,651,655. In one specific, non-limiting example the vaccine composition (medicament) includes at least about 1%, such as about 5%, about 10%, about 20%, about 30%, or about 50% wt/wt inactivated pathogen. As will be apparent to one of ordinary skill in the art, the quantity of pathogen present in the vaccine formulation depends on whether the composition is a liquid or a solid. The amount of inactivated pathogen in a solid composition can exceed that tolerable in a liquid composition. The amount of inactivated pathogen can alternatively be calculated with respect to the comparable amount of a live or inactivated pathogen required to give an immune response. For example, a dosage equivalent in viral particles to from about 10⁶ to about 10¹² plaque forming units (PFU) of live or attenuated virus can be included in a dose of the vaccine composition. Similarly, a vaccine composition can include a quantity of inactivated pathogen (e.g., with RNA or DNA genome), such as virus, equivalent to between about 10³ to about 10¹⁰ live organisms. Alternatively, the dosage can be provided in terms of protein content or concentration. For example, a dose can include from approximately 0.1 μg, such as at least about 0.5 μg protein. For example, a dose can include about 1 μg of an isolated or purified virus or other pathogen up to about 100 μg, or more of a selected pathogen. Although the equivalent doses in infectious units (e.g., PFU) can vary from pathogen to pathogen, the appropriate protein dose can be extrapolated (for example, from PFU) or determined empirically. For example, in a typical preparation, 1 μg of purified vaccinia virus is equivalent to approximately 2×10⁶ PFU. Similar conversions can be determined for any pathogen of interest.

Typically, preparation of a vaccine composition (medicament) entails preparing a pharmaceutical composition that is essentially free of pyrogens, as well as any other impurities that could be harmful to humans or animals. Typically, the pharmaceutical composition contains appropriate salts and buffers to render the components of the composition stable and allow for appropriate processing and presentation of the vaccine antigen by antigen presenting cells. Such components can be supplied in lyophilized form, or can be included in a diluent used for reconstitution of a lyophilized form into a liquid form suitable for administration. Alternatively, where the inactivated pathogen is prepared for administration in a solid state (e.g., as a powder or pellet), a suitable solid carrier is included in the formulation.

Aqueous compositions typically include an effective amount of the inactivated pathogen dispersed (for example, dissolved or suspended) in a pharmaceutically acceptable diluent or aqueous medium. The phrase “pharmaceutically acceptable” refers to molecular entities and compositions that do not produce an adverse, allergic or other undesirable reaction when administered to a human or animal subject. As used herein, “pharmaceutically acceptable carrier” includes any and all solvents, dispersion media, coatings, isotonic and absorption delaying agents and the like. Optionally, a pharmaceutically acceptable carrier or diluent can include an antibacterial, antifungal or other preservative. The use of such media and agents for pharmaceutically active substances is well known in the art. Except insofar as any conventional media or agent is incompatible with production of an immune response by an inactivated pathogen, its use in the immunogenic compositions is contemplated. Supplementary active ingredients also can be incorporated into the compositions. For example, certain pharmaceutical compositions can include the inactivated pathogen in an aqueous diluent, mixed with a suitable surfactant, such as hydroxypropylcellulose. Dispersions also can be prepared in glycerol, liquid polyethylene glycols, and mixtures thereof and in oils. In some cases (for example, when liquid formulations are deemed desirable, or when the lyophilized vaccine composition is reconstituted for multiple doses in a single receptacle), these preparations contain a preservative to prevent the growth of microorganisms.

Pharmaceutically acceptable carriers, excipients and diluents are known to those of ordinary skill in the described, e.g., in Remington's Pharmaceutical Sciences, by E. W. Martin, Mack Publishing Co., Easton, PA, 15th Edition (1975), describes compositions and formulations suitable for pharmaceutical delivery of inactivated pathogens.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example, sodium acetate or sorbitan monolaurate.

For example, the pharmaceutical compositions (medicaments) can include one or more of a stabilizing detergent, a micelle-forming agent, and an oil. Suitable stabilizing detergents, micelle-forming agents, and oils are detailed in U.S. Pat. Nos. 5,585,103; 5,709,860; 5,270,202; and 5,695,770. A stabilizing detergent is any detergent that allows the components of the emulsion to remain as a stable emulsion. Such detergents include polysorbate, 80 (TWEEN80) (Sorbitan-mono-9-octadecenoate-poly(oxy-1,2-ethanediyl; manufactured by ICI Americas, Wilmington, DE), TWEEN 40™, TWEEN 20™, TWEEN 60™, Zwittergent™ 3-12, TEEPOL HB7™, and SPAN 85™. These detergents are usually provided in an amount of approximately 0.05 to 0.5%, such as at about 0.2%. A micelle forming agent is an agent which is able to stabilize the emulsion formed with the other components such that a micelle-like structure is formed. Such agents generally cause some irritation at the site of injection in order to recruit macrophages to enhance the cellular response. Examples of such agents include polymer surfactants described by, e.g., Schmolka, J., Am. Oil. Chem. Soc. 54:110, 1977, and Hunter et al., J. Immunol 129:1244, 1981, and such agents as PLURONIC™ L62LF, L101, and L64, PEG1000, and TETRONIC™ 1501, 150R1, 701, 901, 1301, and 130R1. The chemical structures of such agents are well known in the art. In one embodiment, the agent is chosen to have a hydrophile-lipophile balance (HLB) of between 0 and 2, as defined by Hunter and Bennett, J. Immun. 133:3167, 1984. The agent can be provided in an effective amount, for example between 0.5 and 10%, or in an amount between 1.25 and 5%.

The oil included in the composition is chosen to promote the retention of the pathogen in oil-in-water emulsion, and preferably has a melting temperature of less than 65° C., such that emulsion is formed either at room temperature, or once the temperature of the emulsion is adjusted to room temperature. Examples of such oils include squalene, Squalane, EICOSANE™, tetratetracontane, glycerol, and peanut oil or other vegetable oils. In one specific, non-limiting example, the oil is provided in an amount between 1 and 10%, or between 2.5 and 5%. The oil should be both biodegradable and biocompatible so that the body can break down the oil over time, and so that no adverse effects are evident upon use of the oil.

Optionally, the pharmaceutical compositions or medicaments can include a suitable adjuvant to increase the immune response against the pathogen. As used herein, an “adjuvant” is any potentiator or enhancer of an immune response. The term “suitable” is meant to include any substance which can be used in combination with the selected pathogen to augment the immune response, preferably without producing adverse reactions in the vaccinated subject. Effective amounts of a specific adjuvant may be readily determined so as to optimize the potentiation effect of the adjuvant on the immune response of a vaccinated subject. For example, suitable adjuvants in the context of vaccine formulations include 03%-5% (e.g., 2%) aluminum hydroxide (or aluminum phosphate) and MF-59 oil emulsion (0.5% polysorbate 80 and 0.5% sorbitan trioleate. Squalene (5.0%) aqueous emulsion) is another adjuvant which has been favorably utilized in the context of vaccines. For example, the adjuvant can be a mixture of stabilizing detergents, micelle-forming agent, and oil available under the name Provax® (DEC Pharmaceuticals, San Diego, CA). An adjuvant can also be an immunostimulatory nucleic acid, such as a nucleic acid including a CpG motif. Other adjuvants include mineral, vegetable or fish oil with water emulsions, incomplete Freund's adjuvant, E. coli J5, dextran sulfate, iron sulfate, iron oxide, sodium alginate, Bacto-Adjuvant, certain synthetic polymers such as Carbopol (BF Goodrich Company, Cleveland, Ohio), poly-amino acids and co-polymers of amino acids, saponin, carrageenan, REGRESSIN (Vetrepharm, Athens, Ga.), AVRIDINE (N, N-dioctadecyl-N′,N′-bis(2-hydroxyethyl)-propanediamine), long chain polydispersed.beta. (1,4) linked mannan polymers interspersed with O-acetylated groups (e.g., ACEMANNAN), deproteinized highly purified cell wall extracts derived from non-pathogenic strain of Mycobacterium species (e.g., EQUIMUNE, Vetrepharm Research Inc., Athens Ga.), Mannite monooleate, paraffin oil and muramyl dipeptide. A suitable adjuvant can be selected by one of ordinary skill in the art.

The pharmaceutical compositions (medicaments) can be prepared for use in therapeutic or prophylactic regimens (e.g., vaccines) and administered to human or non-human subjects to elicit an immune response against one or more pathogens. For example, the compositions described herein can be administered to a human (or non-human) subject to elicit a protective immune response against one or more pathogens. To elicit an immune response, a therapeutically effective (e.g., immunologically effective) amount of the inactivated pathogen is administered to a subject, such as a human (or non-human) subject.

A “therapeutically effective amount” is a quantity of a composition used to achieve a desired effect in a subject being treated. For instance, this can be the amount necessary to stimulate an immune response, to prevent infection, to reduce symptoms, or inhibit transmission of a pathogen. When administered to a subject, a dosage will generally be used that will achieve target tissue concentrations (for example, in antigen presenting cells) that is empirically determined to achieve an in vitro effect. Such dosages can be determined without undue experimentation by those of ordinary skill in the art.

An immunogenic composition, such as a vaccine composition containing an inactivated pathogen, can be administered by any means known to one of skill in the art, such as by intramuscular, subcutaneous, or intravenous injection, but even oral, nasal, and transdermal modes are contemplated. In one embodiment, administration is by subcutaneous or intramuscular injection. To extend the time during which the inactivated pathogen is available to stimulate a response, the vaccine composition can be provided as an oily injection, as a particulate system, or as an implant. The particulate system can be a microparticle, a microcapsule, a microsphere, a nanocapsule, or similar particle. A particulate carrier based on a synthetic polymer has been shown to act as an adjuvant to enhance the immune response, in addition to providing a controlled release.

As an alternative to liquid formulations, the composition can be administered in solid form, e.g., as a powder, pellet or tablet. For example, the vaccine composition can be administered as a powder using a transdermal needleless injection device, such as the helium-powered POWDERJECT® injection device. This apparatus uses pressurized helium gas to propel a powder formulation of a vaccine composition, e.g., containing an inactivated pathogen, at high speed so that the vaccine particles perforated the stratum corneum and land in the epidermis.

Polymers can be also used for controlled release. Various degradable and nondegradable polymeric matrices for use in controlled drug delivery are known in the art (Langer, Accounts Chem. Res. 26:537, 1993). For example, the block copolymer, polaxamer 407 exists as a viscous yet mobile liquid at low temperatures but forms a semisolid gel at body temperature. It has shown to be an effective vehicle for formulation and sustained delivery of recombinant interleukin-2 and urease (Johnston, et al., Pharm. Res. 9:425, 1992; and Pec, J. Parent. Sci. Tech. 44 (2): 58, 1990). Alternatively, hydroxyapatite has been used as a microcarrier for controlled release of proteins (Ijntema, et al., Int. J. Pharm. 112:215, 1994). In yet another aspect, liposomes are used for controlled release as well as drug targeting of the lipid-capsulated drug (Betageri, et al., Liposome Drug Delivery Systems, Technomic Publishing Co., Inc., Lancaster, PA, 1993). Numerous additional systems for controlled delivery of therapeutic proteins are known (e.g., U.S. Pat. Nos. 5,055,303; 5,188,837; 4,235,871; 4,501,728; 4,837,028; 4,957,735; and 5,019,369; U.S. Pat. Nos. 5,055,303; 5,514,670; 5,413,797; 5,268,164; 5,004,697; 4,902,505; 5,506,206; 5,271,961; 5,254,342; and 533,096).

In specific, non-limiting examples, the inactivated pathogen is administered to elicit a cellular immune response (e.g., a cytotoxic T lymphocyte (CTL) response). A number of means for inducing cellular responses, both in vitro and in vivo, are known. Lipids have been identified as agents capable of assisting in priming CTL responses in vivo against various antigens. For example, as described in U.S. Pat. No. 5,662,907, palmitic acid residues can be attached to the alpha and epsilon amino groups of a lysine residue and then linked (e.g., via one or more linking residues, such as glycine, glycine-glycine, serine, serine-serine, or the like) to an immunogenic peptide or protein. The lipidated vaccine composition can then be injected directly in a micellar form, incorporated in a liposome, or emulsified in an adjuvant. As another example, E coli lipoproteins, such as tripalmitoyl-S-glycerylcysteinlyseryl-serine can be used to prime tumor specific CTL when covalently attached to an appropriate peptide (see, Deres et al., Nature 342:561, 1989). Further, as the induction of neutralizing antibodies can also be primed with the same molecule conjugated to a peptide which displays an appropriate epitope, two compositions can be combined to elicit both humoral and cell-mediated responses where that is deemed desirable.

Dosages of inactivated pathogen are administered that are sufficient to elicit an immune response, e.g., a protective immune response, in a subject. With respect to viral pathogens, the dosage may be calculated based on the amount of biological matter equivalent to a specified titer of infectious (e.g., virulent or attenuated) virus. For example, a dose equivalent to about 10⁶, or about 10⁷, or about 10⁸, or about 10⁹, or about 10¹⁰, or about 10¹¹ or about 10¹², or even more live virus per dose can be administered to elicit an immune response in a subject. Dosages for viral pathogens may also be calculated based on protein content. In some cases, the dose includes an amount in excess of the amount of a live virus utilized to elicit an immune response, because the inactivated vaccine is incapable of increasing in number after administration into the subject. Typically, the vaccine composition includes additional pharmaceutically acceptable constituents or components. Accordingly, the vaccine composition can include at least about 0.1% wt/wt inactivated pathogen to about 99% wt/wt inactivated pathogen, with the balance of the vaccine composition is made up of pharmaceutically acceptable constituents, such as a one or more pharmaceutically acceptable carrier, pharmaceutically acceptable stabilizer and/or pharmaceutically acceptable diluent. Guidelines regarding vaccine formulation can be found, e.g., in U.S. Pat. Nos. 6,890,542 and 6,651,655. Doses can be calculated based on protein concentration (or infectious units, such as PRJ, of infectious unit equivalents). The optimal dosage can be determined empirically, for example, in preclinical studies in mice and non-human primates, followed by testing in humans in a Phase I clinical trial. Actual methods for preparing administrable compositions will be known or apparent to those skilled in the art and are described in more detail in such publications as Remington's Pharmaceutical Sciences, 19th Ed., Mack Publishing Company, Easton, Pennsylvania, 1995.

Typically, but not always, the vaccine compositions are administered prior to exposure of a subject to a pathogen, e.g., as a vaccine.

It will be apparent that the precise details of the methods or compositions described can be varied or modified without departing from the spirit of the described invention. The following examples are provided to illustrate certain particular features and/or embodiments. These examples should not be construed to limit the invention to the particular features or embodiments described.

### EXAMPLE 1

### Methods

Phylogenetic analysis. The sequence of HA1 subunit antigenic sites for influenza A H1N1, influenza A H3N2 and influenza B have been previously defined (Skowronski, D. M., et al., Low 2012-13 influenza vaccine effectiveness associated with mutation in the egg-adapted H3N2 vaccine strain not antigenic drift in circulating viruses. PLOS One, 2014. 9 (3): p. e92153; Skowronski, D. M., et al., Integrated Sentinel Surveillance Linking Genetic, Antigenic, and Epidemiologic Monitoring of Influenza Vaccine-Virus Relatedness and Effectiveness During the 2013-2014 Influenza Season. J Infect Dis, 2015. 212 (5): p. 726-39). Within each subtype of influenza, an artificial sequence termed; “All Antigenic Sites Combined” (AASC) was defined, which linearly combined the amino acid residue locations for all defined antigenic sites among each influenza subtype. Additionally, a “HA1 Globular Head” sequence was defined for each influenza subtype and included HA1 subunit residues 33-283/284 for influenza A H1N1, or residues 151-250 for influenza A H3N2 or influenza B. For influenza A H3N2 and influenza B sequence analyses, as in the case for the comparisons performed for influenza A H1N1, some sequences were adjusted to account for upstream insertions or deletions among individual HA sequences. All sequences were adjusted to match the HA1 numbering described in Skowronski, D.M., et al., 2015, supra. Using the amino acid sequence of either the AASC or “HA1 Globular Head,” phylogenetic relationships were then investigated using available sequenced strains within each subtype (www.fludb.org), including HA sequences with known sample collection dates, and excluding laboratory strains. In total, 8371 sequences of influenza A H1N1, 9054 sequences of influenza A H3N2, and 3501 sequences of influenza B were analyzed. Influenza B segregates into two distinct lineages, termed Victoria and Yamagata. Prior to further analysis of influenza B, strains were segregated into either of these lineages, resulting in 1705 Victoria lineage strains and 1796 Yamagata lineage strains.

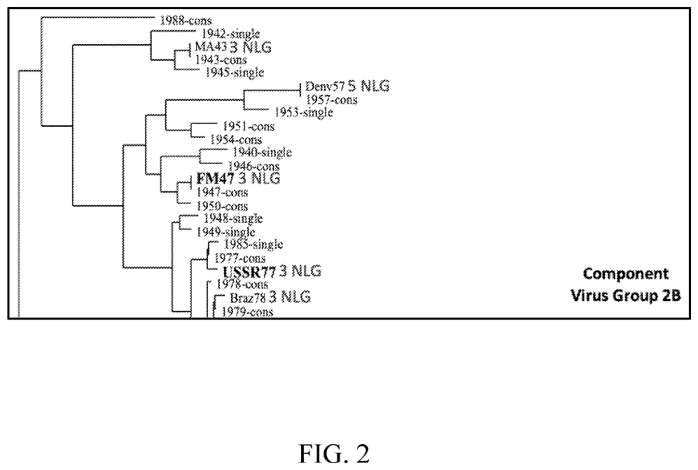

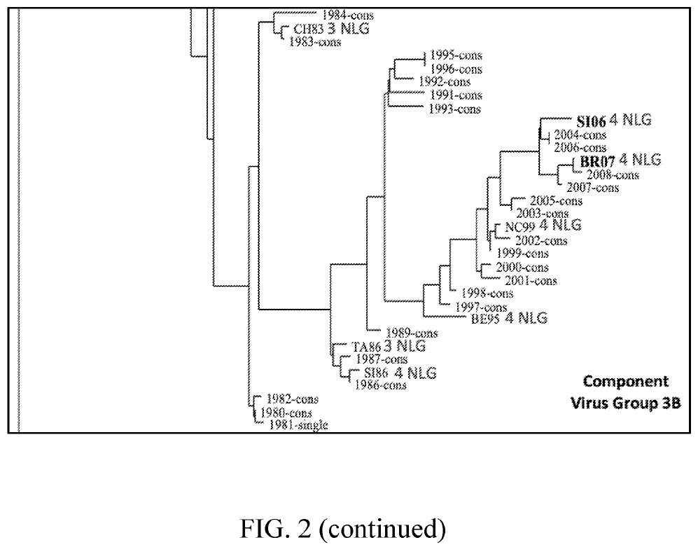

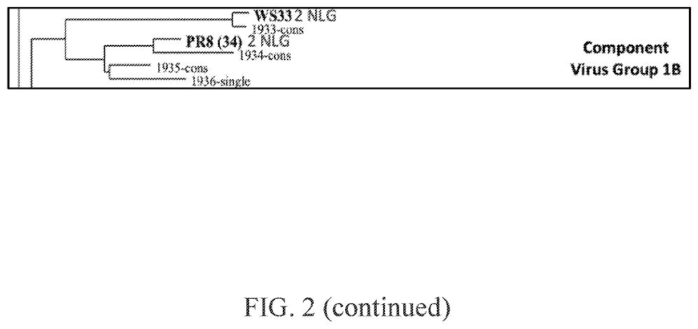

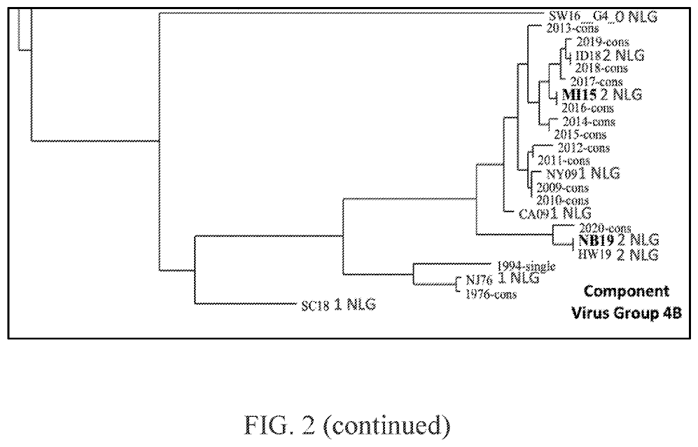

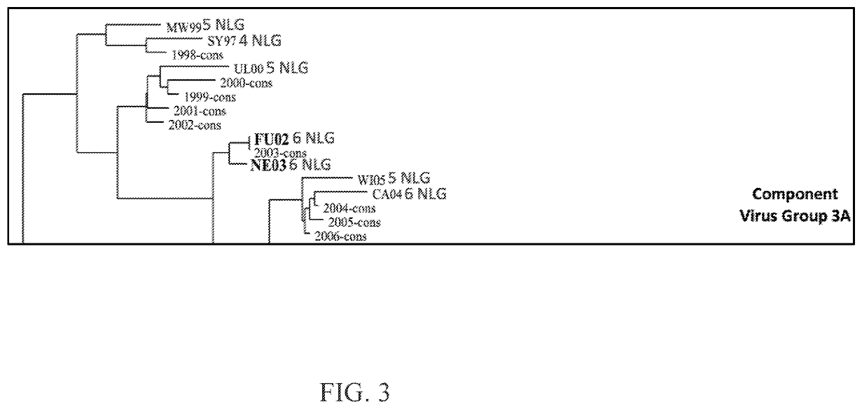

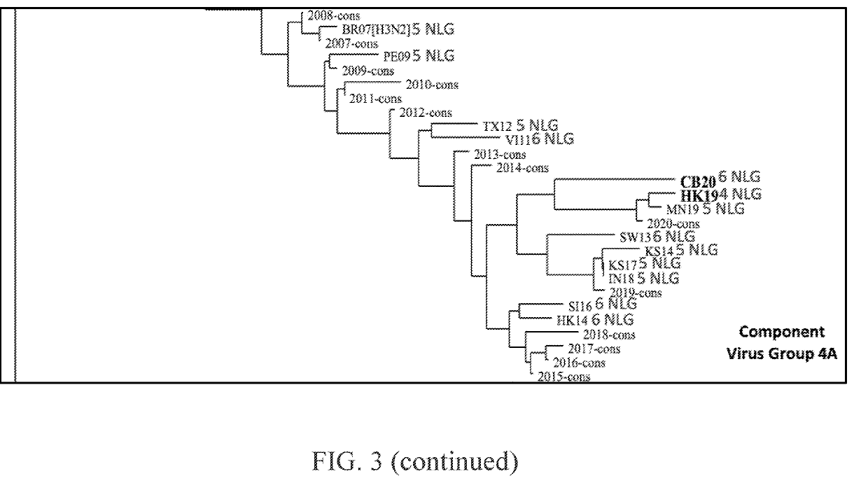

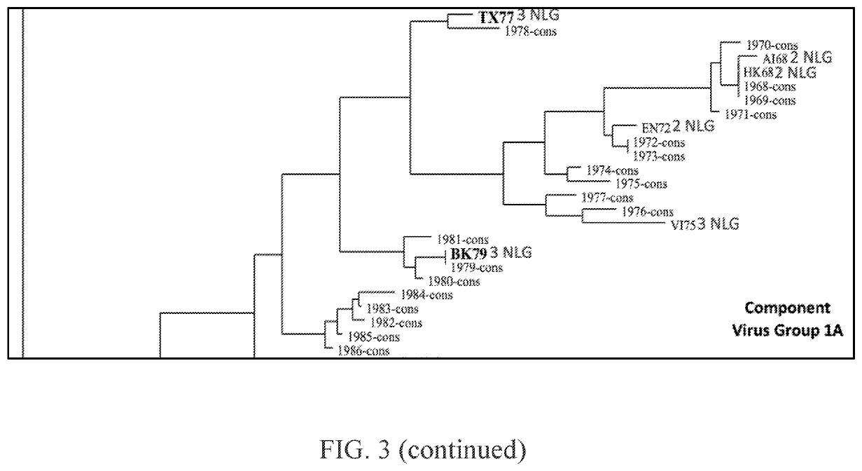

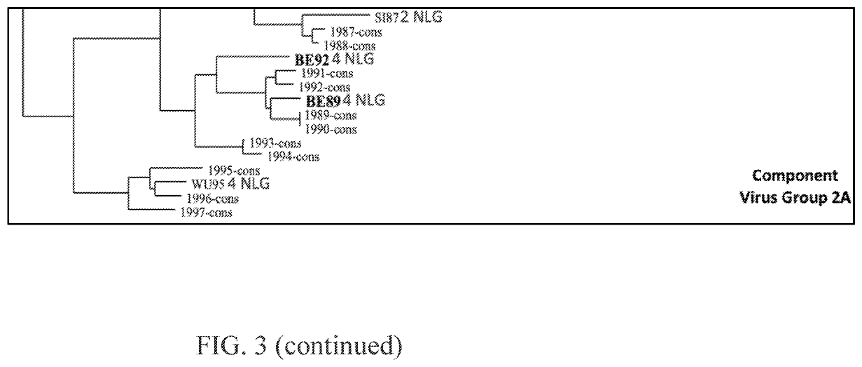

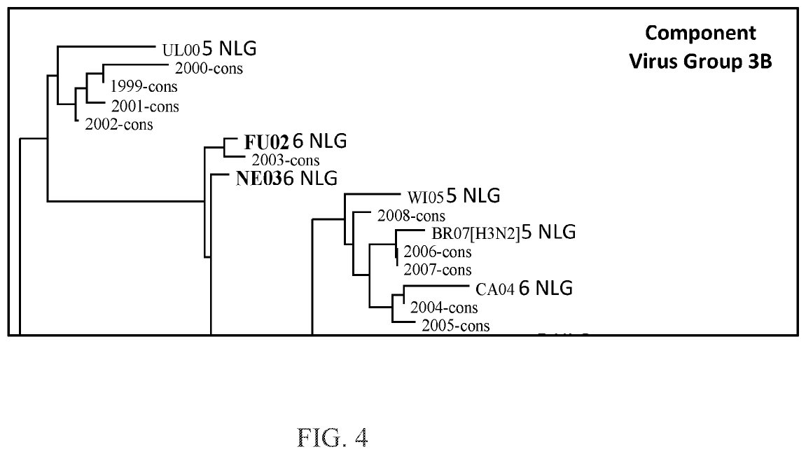

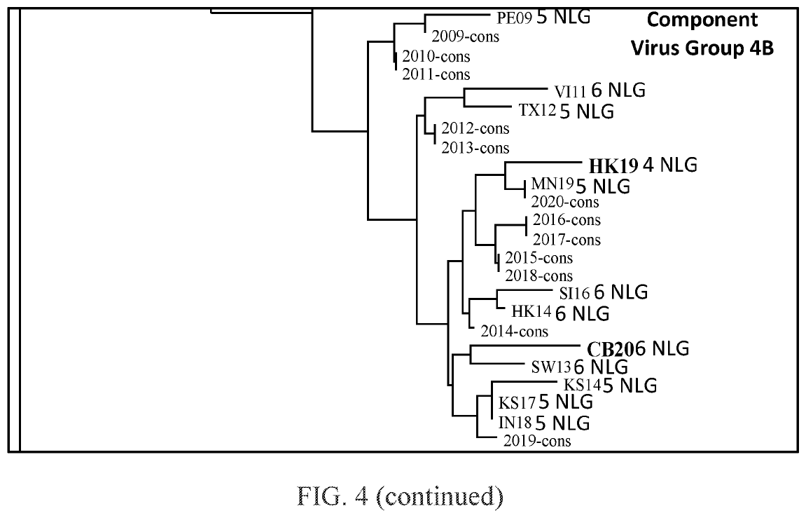

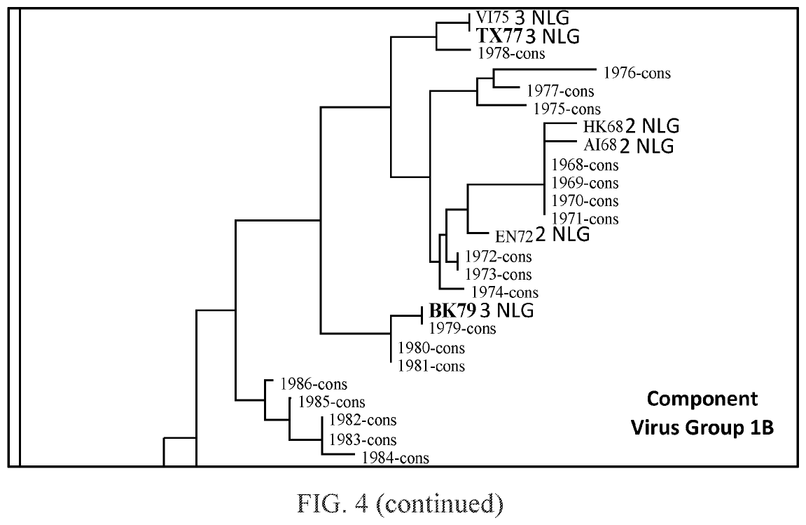

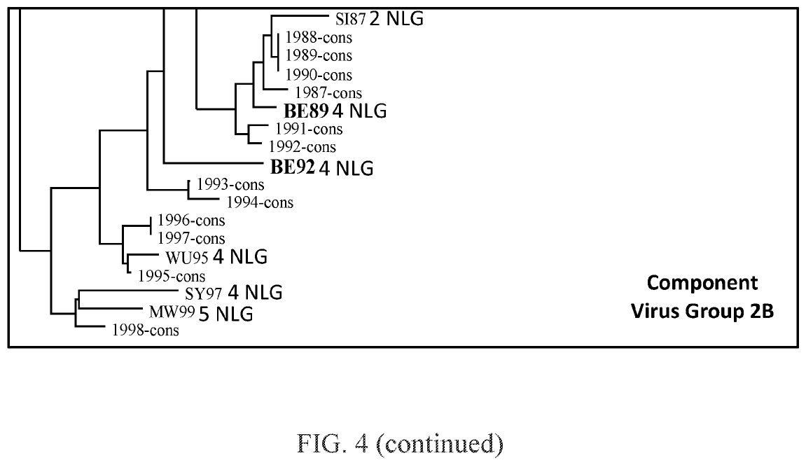

To simplify these large datasets, annual consensus sequences were determined using multiple sequence alignment (https://www.ebi.ac.uk/Tools/msa/clustalo/) followed by consensus alignment (https://www.ebi.ac.uk/Tools/msa/emboss_cons/). In cases where only one sequence was available for a calendar year, that sequence was used as the consensus sequence. For some years, no sequences were available. For influenza A H1N1, starting with the emergence of the 1918 pandemic strain, the years with no sequences include 1919-32, 1937-39, 1941, 1944, 1952, 1955-56, 1958-75, 1990. For influenza H3N2, starting with the emergence of the 1968 pandemic strains, at least one full-length HA sequence has been available for every year since that time. For influenza B, starting with the isolation of the first recorded strain in 1940, years with no full-length HA sequences included 1941-68, 1971, 1974-78, 1981, 1983-85, 1991-92, 1999-2000. For each subtype analysis, additional individual vaccine strains or strains of historical significance were included in the phylogenetic analysis, resulting in a total of 84 HA sequences for influenza A HIN1, 83 HA sequences for influenza A H3N2, and 91 HA sequences for influenza B. Each set of subtype-specific sequences was separately analyzed for their phylogenetic relationship using either the AASC region or the “HA1 Globular Head” region. Using these sequence datasets, six phylogenetic trees were established (https://www.ebi.ac.uk/Tools/msa/clustalo/). Phylogenetic trees were further analyzed and visualized as rectangular phylograms with midpoint roots (Dendroscope v3.7.5). For each of influenza A H1N1 and H3N2, four distinct groupings were observed in the phylogenetic analysis when analyzed according to the AASC and four H1-or H3-specific groupings (H1-Component Virus Group 1A, 2A, 3A, and 4A; or H3-Component Virus Group 1A, 2A, 3A, and 4A,) were defined (FIG. 1 and FIG. 3, respectively). Similarly, for each of influenza A H1N1 and H3N2, four distinct groupings were observed when analyzed using the “HA1 Globular Head” approach and four additional H1-or H3-specific groupings (H1-Component Virus Group 1B, 2B, 3B; and 4B, or H3-Component Virus Group 1B, 2B, 3B, and 4B) were defined (FIG. 2 and FIG. 4, respectively). For either the four H1N1 groups or the four H3N2 groups, the strains encompassed within like-numbered groups 1-4 (e.g., H1-CVG1A and H1-CVG1B; H3-CVG-2A and H3-CVG2B, etc.) were found to be substantially the same.

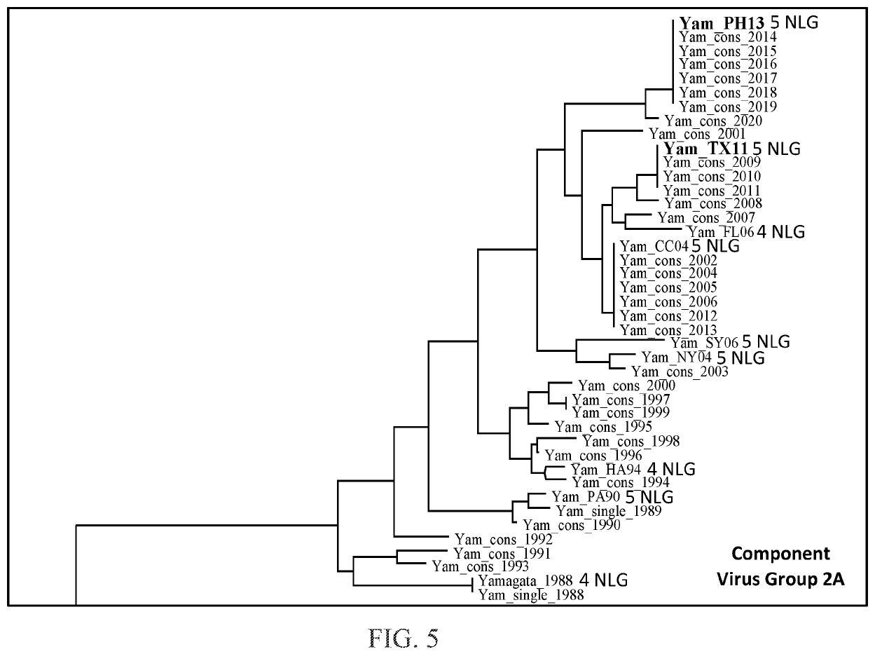

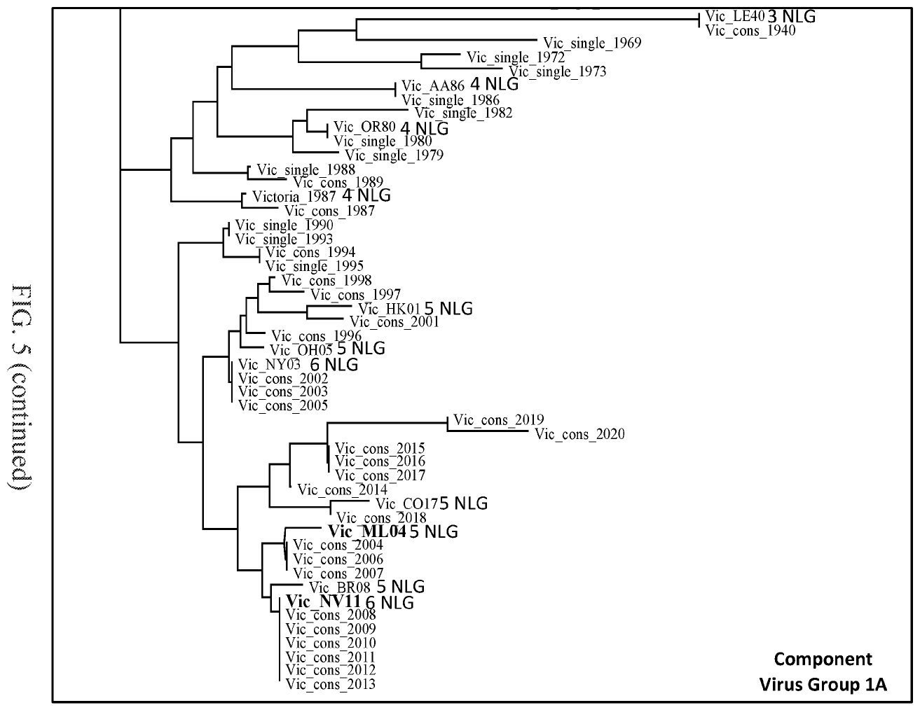

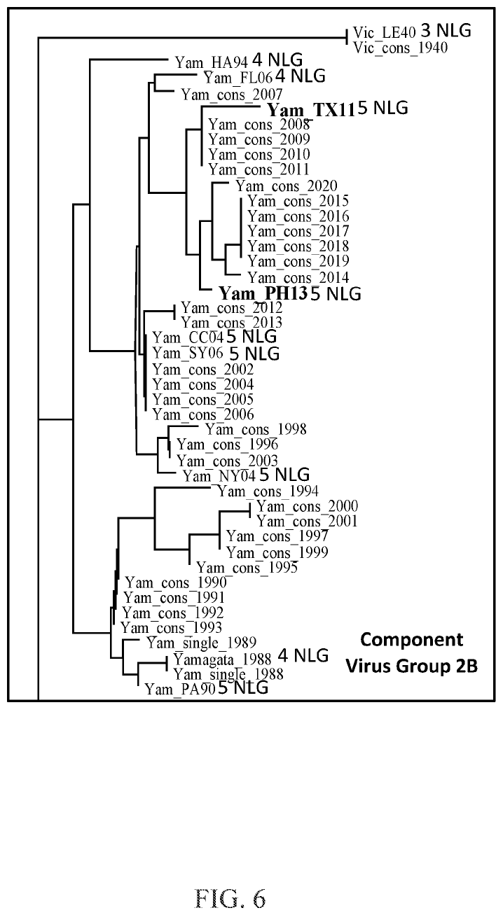

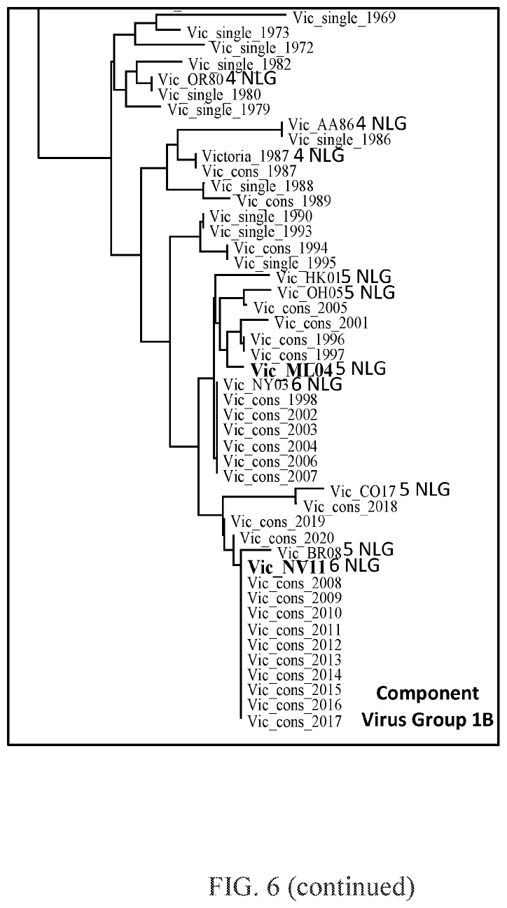

The same analysis was performed for influenza B, where two B-specific groupings (B-Component Virus Group 1A and 2A) based on the AASC sequences, and two B-specific groupings (B-Component Virus Group 1B and 2B) based on the “HA1 Globular Head” sequences, were established (FIG. 5 and FIG. 6, respectively). For the Influenza B groups, the strains encompassed within like-numbered groups 1 and 2 (i.e., Influenza B-CVG1A and Influenza B-CVG1B; and Influenza B-CVG-2A and Influenza B-CVG2B, etc.) were found to be substantially the same.

For each of the 20 Component Virus Groups (H1-CVG-1A-H1-CVG-4A; H1-CVG-1B-H1-CVG-4B; H3-CVG-1A-H3-CVG-4A; H3-CVG-1B-H3-CVG-4B; Influenza B-CVG-1A and Influenza B-CVG-2A; and Influenza B-CVG-1B and Influenza B-CVG-2B) a consensus sequence was determined (https://www.ebi.ac.uk/Tools/msa/emboss_cons/). Two candidate vaccine virus strains were then selected from each like-numbered pair of HIN1, H3N2, and Influenza B Component Virus Groups (20 total candidate strains; 8 Influenza HIN1, 8 Influenza H3N2, and 4 Influenza B) and for each candidate strain, the precent sequence identity between its AASC and “HA1 Globular Head” sequences and the respective consensus AASC and HA1 Globular Head sequences of their respective Component Virus Groups was calculated (Matrix Global Alignment Tool, MatGAT v2.01); see H1N1 Tables 2 and 3 of Example 1; H3N2 Tables 6 and 7 of Example 2; and Tables 10 and 11 of Example 3.

N-linked glycosylation analysis. Using the list (www.fludb.org, Influenza A H1N1 and H3N2 access date: 11 Nov. 2020; Influenza B access date: 22 Jul. 2021) of influenza vaccine strains and strains of historical significance for each influenza subtype (Influenza A H1N1 and H3N2, and Influenza B), predictive N-linked glycosylation analysis was performed across the HA1 globular head region using publicly available software (http://www.cbs.dtu.dk/services/NetNGlyc/).

Virus growth, purification, inactivation and vaccine formulation. Virus vaccine candidates were propagated on either fertilized chicken eggs or Madin-Darby canine kidney (MDCK) cells using standard cell culture techniques. Alternatively, Vero cells may be used. Viruses were harvested and purified by established methodologies including sucrose gradient centrifugation or tangential flow filtration followed by multi-modal size-exclusion chromatography. Each virus strain was inactivated separately using an advanced site-directed oxidation approach (Quintel, B. K., et al., Advanced oxidation technology for the development of a next-generation inactivated West Nile virus vaccine. Vaccine, 2019. 37 (30): p. 4214-4221), based on low concentrations of hydrogen peroxide (H₂O₂) in combination with cupric ions (Cu²⁺ in the form of CuCl₂) complexed with the antiviral compound, methisazone (MZ), as well as a stabilizing concentration of formaldehyde. Specific conditions included 0.005% H₂O₂, 0.125 μM CuCl₂, 20 μM MZ and 0.019% formaldehyde, in a buffer matrix containing a protective level of polyatomic oxyanions (150 mM Na₂HPO₄, pH=7.5) along with other standard buffer components (350 mM NaCl, 10% D-sorbitol and 0.001% polysorbate 80 [Tween80]) for 21-22 hours at room temperature. Residual inactivation components were removed by buffer exchange, and inactivation was confirmed by use of a sensitive co-culture assay. Inactivated virus vaccine antigens were pre-mixed in stoichiometric ratios and adsorbed to 0.20% aluminum hydroxide adjuvant for at least 90 minutes at room temperature.

According to further aspects of the invention, alternative approaches for producing the Influenza A and B vaccines disclosed herein may be used and can include standard approaches to inactivated vaccines such as HydroVax, formaldehyde, β-propiolactone (BPL), or binary ethylenimine (BEI). Vaccination with purified recombinant HA proteins may also be used to elicit protective antiviral antibodies against influenza. For example, immunization of BALB/c mice at day 0 and day 14 with 1 microgram per virus (2 micrograms, total) of a representative influenza virus vaccine (e.g., H3N2, formulated with strains A/Beijing/32/1992 [BE92] and A/Cambodia/e0826360/2020 [CB20]) inactivated using HydroVax (as described herein), BPL (0.1% for 20 hrs at room temperature) or formaldehyde (0.0074% for 1 week at 2-8° C.) were compared to vaccination with purified recombinant BE92 HA and CB20 HA (1 microgram/each) at 14 days after the second vaccination. Geometric mean PRNT50 titers using the HydroVax-based approach reached 190, 1280, and 135 against A/Texas/1/1977 (TX77), BE92 and A/Netherlands/22/2003 (NE03), respectively. Geometric mean PRNT50 titers using the BPL-based approach reached 40, 5120, and 28 against TX77, BE92 and NE03, respectively. Geometric mean PRNT50 titers using the formaldehyde-based approach reached 57, 4305, and 28 against TX77, BE92 and NE03, respectively and geometric mean PRNT50 titers using recombinant HA reached 40, 4305, and 34 against TX77, BE92 and NE03, respectively.

Hemagglutination inhibition (HAI) assay. Serum hemagglutination inhibition (HAI) titers were assessed similar to published WHO methods (World Health Organization., Manual for the laboratory diagnosis and virological surveillance of influenza. 2011, Geneva: World Health Organization. xii, 139 p). Briefly, serum samples were pre-treated with receptor destroying enzyme (RDE) according to manufacturer instructions for 16-20 hours at 37° C. Residual RDE activity was eliminated through heat inactivation at 56° C. for 30 minutes. Serum samples were then pre-adsorbed with phosphate-buffered saline (PBS) rinsed chicken or turkey red blood cells (RBCs) for 30 minutes at ambient room temperature, followed by RBC removal through centrifugation, to limit non-specific RBC binding. Treated serum samples were serially 2-fold diluted in PBS buffer using V-bottom 96-well plates. To 25 μL of each diluted serum sample, 25 μL of pre-titered influenza virus (8 hemagglutination units) was added and allowed to incubate at room temperature for 30 minutes, followed by 50 μL of a PBS-rinsed 1% RBC solution. Hemagglutination reactions were allowed to incubate at room temperature for 45 minutes. The HAI titer was defined as the last serum dilution that maintained full agglutination of the RBCs. Pilot studies with serum samples from unvaccinated rhesus macaques (RM) demonstrated high levels of non-specific HA activity. Therefore, IgG was purified from all RM serum samples according to manufacturer's instructions (Melon Gel IgG spin purification kit, ThermoFisher Scientific) prior to assaying HAI activity. Final HAI titers were normalized based on IgG recoveries through this purification step as assessed by an IgG-specific ELISA performed on pre- and post-purification samples.

Plaque reduction neutralization-50% (PRNT₅₀) assay. Serum plaque reduction neutralization-50% (PRNT₅₀) titers were determined using a plaque reduction assay by incubating 2-fold serial dilutions of heat-inactivated serum with approximately 50 PFU of select influenza strains for 2 hours at 37° C. prior to plating the virus on confluent MDCK cell monolayers. Plaques were developed similar to prior descriptions (Hammarlund, E., et al., A flow cytometry-based assay for quantifying non-plaque forming strains of yellow fever virus. PLOS One, 2012. 7 (9): p. e41707). Briefly, samples were 10-fold serially diluted in growth medium (serum-free EMEM) and dispensed at 0.2 mL per well onto MDCK cell monolayers (˜90% confluent) in 6-well plates. Following a 1-hour incubation at 37° C./5% CO₂, the wells were overlaid with 3 ml of 0.6% agarose in EMEM containing 2.5% fetal bovine serum, 2 mM glutamine and antibiotics and incubated for 3-4 days (depending on the influenza virus strain) at 37° C./5% CO₂. Plates were removed from the incubator and plaques were visualized with crystal violet stain. The PRNT₅₀ titer was defined as the last serum dilution in which at least 50% of input virus was neutralized.

### EXAMPLE 2

### Key Influenza A H1N1 Antigenic Sites

H1N1 Artificial “All Antigenic Sites Combined” sequence. According to aspects of the present invention, as artificial sequence referred to herein as H1N1 “All Antigenic Sites Combined” (“H1-AASC”) corresponding to the linearly combined/conjoined amino acid residue locations of HA1 for all five defined antigenic sites (i.e., the amino acid sequences, in order, of Sa, Sb, Ca₁, Ca₂ and Cb, conjoined in standard amino to carboxyl terminal direction) was used for analysis and comparison between and among influenza virus strains (see Table 1 below).

H1N1 HA1 Globular Head. According to additional aspects of the present invention, an additional approach, complementary to use of the conjoined immunologically defined sites (AASC, which are based on antibody binding), was to define anticipated neutralizing epitopes based on location within a larger contiguous portion of the receptor binding domain of HA1. The globular head of the HA1 subunit binds sialic acid residues on the host target cell during infection and is the most antigenically diverse portion of the HA1 subunit given its surface exposed nature and its role in evading pre-existing neutralizing antibodies “i.e., antigenic drift” in order to maintain active circulation in an immune population. Although the full-length HA has 565/566 amino acid residues, for purposes of the present disclosure using this additional approach, a “H1 HA1 Globular Head” was defined as amino acid residues 33-283/284 based on numbering of the HA1 protein subunit of H1 (see Table 1 below). Note that due to a common insertion in the HA1 subunit among some H1 strains, the numbering system for any residue at HA1 position 127 or higher is, in those strains having the insertion, shifted by a single residue as indicated by “/” to indicate appropriately matched sequence comparisons between and among all the strains compared.

TABLE 1
Location of the H1-AASC sequences/sites in the H1 HA1 hemagglutinin
protein¹, and location the “H1 Globular Head,” referred
to herein, within receptor binding domain of HA1.

| Antigenic Site | |
|---|---|
| (n = number of amino | HA1 Amino Acid |
| acid residues in total) | Residue Number(s)² |
| Sa (n = 13) | 124-125, 152/153-156/157, |
| | 158/159-163/164 |
| Sb (n = 12) | 183/184-194/195 |
| Ca₁ (n = 11) | 165/166-169/170, 202/203- |
| | 204/205, 234/235-236/237 |
| Ca₂ (n = 8) | 136/137-141/142, 220/221-221/222 |
| Cb (n = 6) | 70-75 |
| H1 All Antigenic | 124-125, 152/153-156/157, |
| Sites Combined (H1 | 158/159-163/164, 183/184-194/195, |
| AASC) (n = 50) | 165/166-169/170, 202/203-204/205, |
| | 234/235-236/237, 136/137-141/142, |
| | 220/221-221/222, 70-75 |
| H1 HA1 Globular | 33-283/284 |
| Head (H1 Globular | |
| Head) (n = 251/252) | |

¹Numbering is based on the HA1 subunit as described in Skowronski, D. M., et al., Low 2012-13 influenza vaccine effectiveness associated with mutation in the egg-adapted H3N2 vaccine strain not antigenic drift in circulating viruses. PLoS One, 2014. 9(3): p. e92153.
²Due to a common insertion in the HA1 subunit among H1 strains, the numbering system for any residue at HA1 position 127 or higher may need to be shifted by a single residue as indicated by “/” marks.

Generating phylogenetic trees and computing rooted phylogenetic networks from the trees. The phylogenetic relationship between sequenced H1N1 strains of influenza A was then investigated using either the H1 AASC amino acid sequence or the H1 Globular Head amino acid sequence. Available H1N1 HA protein sequences were collected from a publicly available web resource (www.fludb.org, access date: 11 Nov. 2020) and results were curated to include full HA sequences with known sample collection dates, and to exclude laboratory strains. In total, 8371 HA sequences were initially analyzed. This large dataset was then used to determine annual consensus sequences using multiple sequence alignment (https://www.ebi.ac.uk/Tools/msa/clustalo/) followed by consensus alignment (https://www.ebi.ac.uk/Tools/msa/emboss_cons/). In cases where only one sequence was available for a calendar year, that sequence was used as the consensus sequence for that year. For some years, no H1N1 sequences were available. The sequences of particular individual vaccine strains or strains of historical significance were also included in the phylogenetic analysis. In total, 84 full-length H1N1 HA protein sequences were then used to generate two data sets, one containing sequences of their respective conjoined H1 AASC amino acids (SEQ ID NOS: 1-84) and the other containing their respective “H1 HA1 Globular Head” amino acid sequence (SEQ ID NOS: 89-172). Two respective phylogenetic trees were then established (https://www.ebi.ac.uk/Tools/msa/clustalo/) using these two sequence datasets (FIG. 1 and FIG. 2).

For the phylogenetic tree of FIG. 1, a total of 84 influenza A H1N1 sequences, consisting of either annual consensus sequences or key historical and vaccine strains, were analyzed for their phylogenetic relatedness based on the amino acid sequence of all antigenic sites combined (H1-AASC) as described in the methods. Annual consensus sequences may include years where multiple sequences were available (i.e. Year-cons) or years where only one sequence was available (i.e., Year-single). Historical/vaccine strains included the following: A/South Carolina/1/1918, SC18; A/WSN/1933, WS33; A/PR/8/1934, PR8 (34); A/AA/Marton/1943, MA43; A/Fort Monmouth/1/1947, FM47; A/Denver/57, Denv57; A/New Jersey/1976, NJ76; A/USSR/90/1977, USSR77; A/Brazil/11/1978, Braz78; A/Chile/1/1983, CH83; A/Singapore/6/1986, SI86; A/Taiwan/01/1986, TA86; A/Beijing/262/1995, BE95; A/New Caledonia/20/1999, NC99; A/Solomon Islands/3/2006, SI06; A/Brisbane/59/2007, BR07; A/California/07/2009, CA09; A/New York/18/2009, NY09; A/Michigan/45/2015, MI15; A/swine/Shandong/1207/2016 (H1N1), SW16_(G4); A/Idaho/07/2018, ID18; A/Hawaii/66/2019, HW19; A/Nebraska/14/2019, NB19. Component virus groupings were delineated as shown, and two vaccine strains within each grouping were selected for evaluation as vaccine candidates (bolded). For all historical and vaccine strains, the level of predicted N-linked glycosylations within the HA1 globular head was calculated and is shown next to each strain.

For the phylogenetic tree of FIG. 2, a total of 84 influenza A H1N1 sequences, consisting of either annual consensus sequences or key historical and vaccine strains, were analyzed for their phylogenetic relatedness based on the HA1 globular head as described in the methods. Annual consensus sequences may include years where multiple sequences were available (i.e., Year-cons) or years where only one sequence was available (i.e., Year-single). Historical/vaccine strains included the following: A/South Carolina/1/1918, SC18; A/WSN/1933, WS33; A/PR/8/1934, PR8 (34); A/AA/Marton/1943, MA43; A/Fort Monmouth/1/1947, FM47; A/Denver/57, Denv57; A/New Jersey/1976, NJ76; A/USSR/90/1977, USSR77; A/Brazil/11/1978, Braz78; A/Chile/1/1983, CH83; A/Singapore/6/1986, SI86; A/Taiwan/01/1986, TA86; A/Beijing/262/1995, BE95; A/New Caledonia/20/1999, NC99; A/Solomon Islands/3/2006, SI06; A/Brisbane/59/2007, BR07; A/California/07/2009, CA09; A/New York/18/2009, NY09; A/Michigan/45/2015, MI15; A/swine/Shandong/1207/2016 (H1N1), SW16_(G4); A/Idaho/07/2018, ID18; A/Hawaii/66/2019, HW19; A/Nebraska/14/2019, NB19. Component virus groupings were delineated as shown, and two vaccine strains within each grouping were selected for evaluation as vaccine candidates (bolded). For all historical and vaccine strains, the level of predicted N-linked glycosylations within the HA1 globular head was calculated and is shown next to each strain.

The phylogenetic trees were further analyzed by computing rooted phylogenetic networks from the trees (Dendroscope v3.7.5) and visualized as rectangular phylograms with midpoint roots. Following this analysis, four distinct strain groupings were identified in both phylogenetic trees. For the H1 AASC tree/network these were defined as H1 Component Virus Groups 1A-4A (H1-CVG1A-H1-CVG4A), and for the “H1 Globular Head” tree/network as analogous H1 Component Virus Groups 1B-4B (H1-CVG1B-H1-CVG4B), where the viral strains encompassed by analogous groups (e.g., by H1-CVG1A and H1-CVG1B, etc.) were substantially the same.

Two exemplary virus strains were selected from each of the four H1-AASC component groups (H1-CVG1A-H1-CVG4A), and from each of the four “H1 Globular Head” groups (H1-CVG1B-H1-CVG4B), to test as potential vaccine candidates. The selected strains were: A/WSN/1933 (WS33) (having full HA SEQ ID NO:177) and A/PR/8/1934 (PR8 (34)) (having full HA SEQ ID NO:178 (both encompassed by either H1-CVG1A or H1-CVG1B); A/Fort Monmouth/1/1947 (FM47) (having full HA SEQ ID NO: 179) and A/USSR/90/1977 (USSR77) (having full HA SEQ ID NO:180) (both encompassed by either H1-CVG2A or H1-CVG2B); A/Solomon Islands/3/2006 (SI06) (having full HA SEQ ID NO:181) and A/Brisbane/59/2007 (BR07) (having full HA SEQ ID NO: 182) (both encompassed by either H1-CVG3A or H1-CVG3B); A/Michigan/45/2015 (MI15) (having full HA SEQ ID NO:183) and A/Nebraska/14/2019 (NB19) (full HA SEQ ID NO: 184) (both encompassed by either H1-CVG4A or H1-CVG4B).

The exemplary diverse test strains were clinically isolated influenza strains that could be developed into vaccine candidates, and provided a breadth of sequence diversity within the disclosed individual component virus groups (H1-CVG1A-H1-CVG4A and H1-CVG1B-H1-CVG4B). Respective consensus sequences were also determined for each of the H1-CVG1A-H1-CVG4A (H1-AASC) groups (SEQ ID NOS: 85-88, respectively), and for each of the H1-CVG1B-H1-CVG4B (“H1 HA1 Globular Head”) groups (SEQ ID NOS: 173-176, respectively), in each case based on all of the viral HA sequences used to define the respective groupings.

Sequence comparisons (% identity) were then made between the AASC and Globular Head sequences of each test strain and the consensus sequences their respective groupings; H1 CVG1A-H1-CVG4A (H1 AASC groupings), and H1-CVG1B-H1-CVG4B (“H1 Globular Head” groupings), as shown below in Table 2 (H1-AASC comparison) and Table 3 (“H1 Globular Head” comparison), respectively.

TABLE 2
Sequence comparison (% identity) based on all antigenic sites combined (H1-AASC) sequences.

| | Component Virus Groups | | | | Select Virus Strains | | | | | | | |
|---|---|---|---|---|---|---|---|---|---|---|---|---|
| | CVG1A | CVG2A | CVG3A | CVG4A | WS33 | PR8 | FM47 | USSR77 | SI06 | BR07 | MI15 | NB19 |
| | (SEQ | (SEQ | (SEQ | (SEQ | (HA SEQ | (HA SEQ | (HA SEQ | (HA SEQ | (HA SEQ | (HA SEQ | (HA SEQ | (HA SEQ |
| | ID NO: | ID NO: | ID NO: | ID NO: | ID NO: | ID NO: | ID NO: | ID NO: | ID NO: | ID NO: | ID NO: | ID NO: |
| | 85) | 86) | 87) | 88) | 177) | 178) | 179) | 180) | 181) | 182) | 183) | 184) |
| CVG1A | 100 | 56 | 56 | 64 | 84 | 82 | 62 | 54 | 52 | 52 | 64 | 62 |
| (SEQ ID | | | | | | | | | | | | |
| NO: 85) | | | | | | | | | | | | |
| CVG2A | | 100 | 74 | 44 | 52 | 56 | 90 | 98 | 70 | 72 | 42 | 40 |
| (SEQ ID | | | | | | | | | | | | |
| NO: 86) | | | | | | | | | | | | |
| CVG3A | | | 100 | 46 | 52 | 54 | 70 | 72 | 92 | 94 | 44 | 42 |
| (SEQ ID | | | | | | | | | | | | |
| NO: 87) | | | | | | | | | | | | |
| CVG4A | | | | 100 | 58 | 56 | 46 | 42 | 46 | 46 | 98 | 88 |
| (SEQ ID | | | | | | | | | | | | |
| NO: 88) | | | | | | | | | | | | |

TABLE 3
Sequence Comparison (% identity) Based on “H1 Globular Head” sequences.

| | Component Virus Groups | | | | Select Virus Strains | | | | | | | |
|---|---|---|---|---|---|---|---|---|---|---|---|---|
| | CVG1B | CVG2B | CVG3B | CVG4B | WS33 | PR8 | FM47 | USSR77 | SI06 | BR07 | MI15 | NB19 |
| | (SEQ | (SEQ | (SEQ | (SEQ | (HA SEQ | (HA SEQ | (HA SEQ | (HA SEQ | (HA SEQ | (HA SEQ | (HA SEQ | (HA SEQ |
| | ID NO: | ID NO: | ID NO: | ID NO: | ID NO: | ID NO: | ID NO: | ID NO: | ID NO: | ID NO: | ID NO: | ID NO: |
| | 173) | 174) | 175) | 176) | 177) | 178) | 179) | 180) | 181) | 182) | 183) | 184) |
| CVG1B | 100 | 88 | 81 | 73 | 91 | 94 | 87 | 84 | 79 | 78 | 73 | 72 |
| (SEQ ID | | | | | | | | | | | | |
| NO: 173) | | | | | | | | | | | | |
| CVG2B | | 100 | 88 | 69 | 83 | 87 | 98 | 96 | 84 | 84 | 69 | 68 |
| (SEQ ID | | | | | | | | | | | | |
| NO: 174) | | | | | | | | | | | | |
| CVG3B | | | 100 | 68 | 79 | 82 | 87 | 89 | 93 | 93 | 67 | 67 |
| (SEQ ID | | | | | | | | | | | | |
| NO: 175) | | | | | | | | | | | | |
| CVG4B | | | | 100 | 71 | 72 | 69 | 66 | 68 | 67 | 98 | 96 |
| (SEQ ID | | | | | | | | | | | | |
| NO: 176) | | | | | | | | | | | | |

N-linked glycosylation (NLG) sites. In addition to evading host immune responses through mutation of surface-exposed HA amino acid residues, influenza strains can also mutate their number of N-linked glycosylation (NLG) sites (Sun, S., et al., Glycosylation site alteration in the evolution of influenza A (H1N1) viruses. PLoS One, 2011. 6 (7): p. e22844). N-linked glycosylations can occur at asparagine amino acid residues (three-letter abbreviation=Asn, single letter abbreviation=N) and are generally found as part of the Asn-X-Ser/Thr consensus sequence, where X is any amino acid except proline. Pandemic strains of influenza often have a low level of HA glycosylation, which typically increases in subsequent years of transmission to evade the immunodominant host immune response. In addition to selecting clinically isolated strains that provided wide diversity across the defined Component Virus Groups (FIGS. 1 and 2) the selected test viruses included those having a higher degree of NLG (e.g., ≥2 predicted NLG sites). According to particular aspects of the present invention, inclusion of NLG sites in the H1 Globular Head Region may be used to further improve or tailor immunogenicity and/or immune response with the disclosed vaccines. Using the list (www.fludb.org, access date: 11 Nov. 2020) of influenza vaccine strains and strains of historical significance, predictive N-linked glycosylation analysis was performed using publicly available software (http://www.cbs.dtu.dk/services/NetNGlyc/) to compare the level of predicted NLG modifications across strains (Table 4). The total predicted NLG varied from as few as 0, to as many as 5 predicted NLG sites across the H1 HA1 Globular Head. Particular exemplary test strains selected for vaccine development (see Tables 2 and 3) ranged from 2-4 predicted NLG per H1 HA1 Globular Head (see strains in bold-face type in column 1 of Table 4).

TABLE 4
Examples of predicted Influenza A H1N1 N-linked
glycosylations (NLG) in the H1 HA1 Globular Head

| | Potential N-Linked Glycosylation Sites¹ | | | | | | | | | |
|---|---|---|---|---|---|---|---|---|---|---|
| Influenza | | | | | | | | | | Total |
| Strain² | 54 | 73 | 87 | 124/125 | 126/127 | 154/155 | 159/160 | 161/162 | 268/269 | NLG |
| SC18 | | | Yes | | | | | | | 1 |
| WS33 | | | | Yes | | | | | Yes | 2 |
| PR8 (34) | | | | | Yes | | | | Yes | 2 |
| MA43 | | | Yes | | Yes | | | Yes | | 3 |
| FM47 | | | Yes | | Yes | Yes | | | | 3 |
| Denv57 | | Yes | Yes | Yes | | Yes | Yes | | | 5 |
| NJ76 | | | Yes | | | | | | | 1 |
| USSR77 | | | Yes | | Yes | | Yes | | | 3 |
| Braz78 | | | Yes | | Yes | | Yes | | | 3 |
| CH83 | | | Yes | | Yes | | Yes | | | 3 |
| TA86 | Yes | | Yes | Yes | | | | | | 3 |
| SI86 | Yes | | Yes | Yes | | | Yes | | | 4 |
| BE95 | Yes | | Yes | Yes | | | Yes | | | 4 |
| NC99 | Yes | | Yes | Yes | | | Yes | | | 4 |
| SI06 | Yes | | Yes | Yes | | | Yes | | | 4 |
| BR07 | Yes | | Yes | Yes | | | Yes | | | 4 |
| NY09 | | | Yes | | | | | | | 1 |
| CA09 | | | Yes | | | | | | | 1 |
| MI15 | | | Yes | | | | | Yes | | 2 |
| ID18 | | | Yes | | | | | Yes | | 2 |
| NB19 | | | Yes | | | | | Yes | | 2 |
| HW19 | | | Yes | | | | | Yes | | 2 |
| SW16_(G4) | | | | | | | | | | 0 |

¹The amino acid residue position of predicted N-linked glycosylation sites (http://www.cbs.dtu.dk/services/NetNGlyc/) and renumbered according to the HA1 protein subunit. Only those potential sites that fall within the anticipated HA1 Globular Head region are shown.
²Strains chosen as exemplary vaccine candidates are shown in bold.

### EXAMPLE 3

### Key Influenza A H3N2 Antigenic Sites

H3 Artificial “All Antigenic Sites Combined” sequence. Similar to the H1N1 subtype of Influenza A, the H3N2 subtype also has five defined antigenic sites, termed sites A-E (Skowronski, D. M., et al., Integrated Sentinel Surveillance Linking Genetic, Antigenic, and Epidemiologic Monitoring of Influenza Vaccine-Virus Relatedness and Effectiveness During the 2013-2014 Influenza Season. J Infect Dis, 2015. 212 (5): p. 726-39) (Table 5). In analogy with the approach used with H1N1, an artificial sequence referred to herein as “All Antigenic Sites Combined (H3-AASC)” which linearly combines/conjoins the amino acid residue locations for all five defined H3 antigenic sites (i.e., the amino acid sequences, in order, of A, B, C, D and E, conjoined in standard amino to carboxyl terminal direction) was used for comparison between and among H3N2 influenza virus strains (Table 5 below).

Influenza A H3N2 HA1 Globular Head (H3 HA1 Globular Head). As with H1N1, an additional approach, complementary to use of the conjoined immunologically defined sites that are based on antibody binding, was to define anticipated neutralizing epitopes based on location within a larger contiguous portion of the receptor binding domain of the H3 HA1 protein. For this analysis with H3N2, the H3 HA1 Globular Head of the HA was defined as amino acid residues 151-250 based on numbering of the H3 HA1 protein subunit (Table 5 below).

As in the above Influenza A H1N1 analysis, for both Influenza A H3N2 sequence analyses/comparisons some sequences were adjusted to account for upstream insertions or deletions among individual HA sequences. All sequences were adjusted to match the H3 HA1 numbering described in (Skowronski, D. M., et al., supra).

TABLE 5
Location of the H3-AASC sequences/sites in the H3 HA1 hemagglutinin
protein¹, and location the “H3-HA1 Globular Head,”
referred to herein, within receptor binding domain of H3 HA1

| Antigenic Site | |
|---|---|
| (n = number of amino | |
| acid residues in total) | Amino Acid Residue Number(s) |
| A (n = 19) | 122, 124, 126, 130-133, 135, 137, |
| | 138, 140, 142-146, 150, 152, 168 |
| B (n = 22) | 128, 129, 155-160, 163-165, 186-190, |
| | 192-194, 196-198 |
| C (n = 27) | 44-48, 50, 51, 53, 54, 273, 275, 276, |
| | 278-280, 294, 297, 299, 300, 304, 305, |
| | 307-312 |
| D (n = 41) | 96, 102, 103, 117, 121, 167, 170-177, |
| | 179, 182, 201, 203, 207-209, 212-219, |
| | 226-230, 238, 240, 242, 244, 246-248 |
| E (n = 22) | 57, 59, 62, 63, 67, 75, 78, 80-83, |
| | 86-88, 91, 92, 94, 109, 260-262, 265 |
| H3 All Antigenic Sites | 122, 124, 126, 130-133, 135, 137, 138, |
| Combined (H3 AASC) | 140, 142-146, 150, 152, 168, 128, 129, |
| (n = 131) | 155-160, 163-165, 186-190, 192-194, |
| | 196-198, 44-48, 50, 51, 53, 54, 273, |
| | 275, 276, 278-280, 294, 297, 299, 300, |
| | 304, 305, 307-312, 96, 102, 103, 117, |
| | 121, 167, 170-177, 179, 182, 201, 203, |
| | 207-209, 212-219, 226-230, 238, 240, |
| | 242, 244, 246-248, 57, 59, 62, 63, 67, |
| | 75, 78, 80-83, 86-88, 91, 92, 94, 109, |
| | 260-262, 265 |
| H3 HA1 Globular Head | 151-250 |
| (n = 100) | |

¹Numbering is based on the HA1 subunit as described in (Skowronski, D. M., et al., supra).

Generating phylogenetic trees, and computing rooted phylogenetic networks from the trees. Using the same approach detailed for H1N1 above, the phylogenetic relationship between sequenced H3N2 strains of influenza A was then investigated using either the H3 AASC amino acid sequence or the H3 Globular Head amino acid sequence. Available H3N2 HA protein sequences were collected from a publicly available web resource (www.fludb.org, access date: 11 Nov. 2020) and results were curated to include full HA sequences with known sample collection dates and to exclude laboratory strains. In total, 9,054 HA sequences were initially analyzed. This large dataset was then used to determine annual consensus sequences using multiple sequence alignment (https://www.ebi.ac.uk/Tools/msa/clustalo/) followed by consensus alignment (https://www.ebi.ac.uk/Tools/msa/emboss_cons/). In cases where only one sequence was available for a calendar year, that sequence was used as the consensus sequence. The sequences of particular individual vaccine strains or strains of historical significance were also included in the phylogenetic analysis. In total, 83 full-length HA protein sequences were then used to generate two data sets, one containing their respective conjoined H3-AASC amino acid sequence (SEQ ID NOS: 185-267) and the other containing their respective “H3 HA1 Globular Head” region (SEQ ID NOS: 272-384). Two respective phylogenetic trees were then established (https://www.ebi.ac.uk/Tools/msa/clustalo/) using these two sequence datasets (FIG. 3 and FIG. 4).

For the phylogenetic tree of FIG. 3, a total of 83 influenza A H3N2 sequences, consisting of either annual consensus sequences or key historical and vaccine strains, were analyzed for their phylogenetic relatedness based on the amino acid sequence of all antigenic sites combined (H3-AASC) as described in the methods. Annual consensus sequences may include years where multiple sequences were available (i.e. Year-cons) or years where only one sequence was available (i.e., Year-single). Historical/vaccine strains included the following: A/Aichi/2/68, AI68; A/Hong Kong/1/1968, HK68; A/England/42/1972, EN72; A/Victoria/3/1975, VI75; A/Texas/1/1977, TX77; A/Bangkok/1/1979, BK79; A/Sichuan/2/1987, SI87; A/Beijing/353/1989, BE89; A/Beijing/32/1992, BE92; A/Wuhan/359/95, WU95; A/Sydney/5/1997, SY97; A/Moscow/10/99, MW99; A/Ulan Ude/01/2000, UL00; A/Fujian/411/2002, FU02; A/Netherlands/22/2003, NE03; A/California/7/2004, CA04; A/Wisconsin/67/2005, WI05; A/Brisbane/10/2007, BR07[H3N2]; A/Perth/16/2009, PE09; A/Victoria/361/2011, VI11; A/Texas/50/2012, TX12; A/Switzerland/9715293/2013, SW13; A/Kansas/14/2017, KS14; A/Hong Kong/4801/2014, HK14; A/Singapore/INFIMH-16-0019/2016, SI16; A/Kansas/14/2017, KS17; A/Indiana/08/2018, IN18; A/Hong Kong/2671/2019, HK19; A/Minnesota/41/2019, MN19; A/Cambodia/e0826360/2020, CB20. Component virus groupings were delineated as shown, and two vaccine strains within each grouping were selected for evaluation as vaccine candidates (bolded). For all historical and vaccine strains, the level of predicted N-linked glycosylations within the HA1 globular head was calculated and is shown next to each strain.

For the phylogenetic tree of FIG. 4, a total of 83 influenza A H3N2 sequences, consisting of either annual consensus sequences or key historical and vaccine strains, were analyzed for their phylogenetic relatedness based on the HA1 globular head as described in the methods. Annual consensus sequences may include years where multiple sequences were available (i.e. Year-cons) or years where only one sequence was available (i.e., Year-single). Historical/vaccine strains included the following: A/Aichi/2/68, AI68; A/Hong Kong/1/1968, HK68; A/England/42/1972, EN72; A/Victoria/3/1975, VI75; A/Texas/1/1977, TX77; A/Bangkok/1/1979, BK79; A/Sichuan/2/1987, SI87; A/Beijing/353/1989, BE89; A/Beijing/32/1992, BE92; A/Wuhan/359/95, WU95; A/Sydney/5/1997 SY97; A/Moscow/10/99, MW99; A/Ulan Ude/01/2000, UL00; A/Fujian/411/2002, FU02; A/Netherlands/22/2003, NE03; A/California/7/2004, CA04; A/Wisconsin/67/2005, WI05; A/Brisbane/10/2007, BR07[H3N2]; A/Perth/16/2009, PE09; A/Victoria/361/2011, VI11; A/Texas/50/2012, TX12; A/Switzerland/9715293/2013, SW13; A/Kansas/14/2017, KS14; A/Hong Kong/4801/2014, HK14; A/Singapore/INFIMH-16-0019/2016, SI16; A/Kansas/14/2017, KS17; A/Indiana/08/2018, IN18; A/Hong Kong/2671/2019, HK19; A/Minnesota/41/2019, MN19; A/Cambodia/e0826360/2020, CB20. Component virus groupings were delineated as shown, and two vaccine strains within each grouping were selected for evaluation as vaccine candidates (bolded). For all historical and vaccine strains, the level of predicted N-linked glycosylations within the HA1 globular head was calculated and is shown next to each strain.

The phylogenetic trees were further analyzed by computing rooted phylogenetic networks from the trees (Dendroscope v3.7.5) and visualized as rectangular phylograms with midpoint roots. Following this analysis, four distinct strain groupings were identified in both phylogenetic trees. For the “H3-AASC” tree/network, these were defined as H3 Component Virus Groups 1A-4A (H3-CVG1A-H3-CVG4A), and for the “H3 HA1 Globular Head” tree/network as analogous H3 Component Virus Groups 1B-4B (H3-CVG1B-H3-CVG4B), where the viral strains encompassed by analogous groups (e.g., by H3-CVG1A and H3-CVG1B, etc.) were substantially the same.

Using these groupings as a guide, two exemplary virus strains were selected from each of the four H3 AASC component groups (H3-CVG1A-H3-CVG4A), and from each of the four “H3 Globular Head” groups (H3-CVG1B-H3-CVG4B), to test as potential vaccine candidates. The selected strains were: A/Texas/1/1977 (TX77) (having full HA SEQ ID NO: 359) and A/Bangkok/1/1979 (BK79) (full HA SEQ ID NO:360) (both encompassed by either H3-CVG1A or H3-CVG1B); A/Beijing/353/1989 (BE89) (full HA SEQ ID NO:361) and A/Beijing/32/1992 (BE92) (full HA SEQ ID NO:362) (both encompassed by either H3-CVG2A or H3-CVG2B); A/Fujian/411/2002 (FU02) (full HA SEQ ID NO:364) and 20) A/Netherlands/22/2003 (NE03) (full HA SEQ ID NO:363) (both encompassed by either H3-CVG3A or H3-CVG3B); A/Hong Kong/2671/2019 (HK19) (full HA SEQ ID NO:365) and A/Cambodia/e0826360/2020 (CB2) (full HA SEQ ID NO:366) (both encompassed by either H3 CVG4A or H4 CVG4B).

The exemplary diverse test strains were clinically isolated influenza strains that could be developed into vaccine candidates, and provided a breadth of sequence diversity within the disclosed individual component virus groups (H3-CVG1A-H3-CVG4A and H3-CVG1B-H3-CVG4B). Respective consensus sequences were also determined for each of the H3-CVG1A-H3-CVG4A (H3-AASC) groups (SEQ ID NOS: 268-271, respectively), and for each of the H3-CVG1B-H3-CVG4B (“H3 HA1 Globular Head”) groups (SEQ ID NOS: 355-358, respectively), in each case based on all the viral H3 HA sequences used to define the respective groupings.

Sequence comparisons (% identity) were then made between the AASC and Globular Head sequences of each test strain and the consensus sequences their respective groupings; H3-CVG1A-H3-CVG4A (H3-AASC groupings), and H3-CVG2B-H3-CVG4B (“H3 HA1 Globular Head” groupings), as shown below in Table 6 (H3 AASC comparison) and Table 7 (“H3 Globular Head” comparison), respectively.

TABLE 6
Sequence comparison (% identity) based on all antigenic sites combined (H3-AASC) sequences.

| | Component Virus Groups | | | | Select Virus Strains | | | | | | | |
|---|---|---|---|---|---|---|---|---|---|---|---|---|
| | CVG1A | CVG2A | CVG3A | CVG4A | TX77 | BK79 | BE89 | BE92 | FU02 | NE03 | HK19 | CB20 |
| | (SEQ | (SEQ | (SEQ | (SEQ | (HA SEQ | (HA SEQ | (HA SEQ | (HA SEQ | (HA SEQ | (HA SEQ | (HA SEQ | (HA SEQ |
| | ID NO: | ID NO: | ID NO: | ID NO: | ID NO: | ID NO: | ID NO: | ID NO: | ID NO: | ID NO: | ID NO: | ID NO: |
| | 268) | 269) | 270) | 271) | 359) | 360) | 361) | 362) | 364) | 363) | 365) | 366) |
| CVG1A | 100 | 77 | 68 | 65 | 92 | 88 | 77 | 75 | 69 | 69 | 62 | 62 |
| (SEQ ID | | | | | | | | | | | | |
| NO: 268) | | | | | | | | | | | | |
| CVG2A | | 100 | 81 | 72 | 82 | 84 | 97 | 95 | 78 | 78 | 69 | 71 |
| (SEQ ID | | | | | | | | | | | | |
| NO: 269) | | | | | | | | | | | | |
| CVG3A | | | 100 | 83 | 70 | 72 | 81 | 81 | 95 | 93 | 79 | 82 |
| (SEQ ID | | | | | | | | | | | | |
| NO270) | | | | | | | | | | | | |
| CVG4A | | | | 100 | 66 | 66 | 70 | 71 | 88 | 88 | 89 | 89 |
| (SEQ ID | | | | | | | | | | | | |
| NO: 271) | | | | | | | | | | | | |

TABLE 7
Sequence Comparison (% identity) Based on “H3 HA1 Globular Head” sequences.

| | Component Virus Groups | | | | Select Virus Strains | | | | | | | |
|---|---|---|---|---|---|---|---|---|---|---|---|---|
| | CVG1B | CVG2B | CVG3B | CVG4B | TX77 | BK79 | BE89 | BE92 | FU02 | NE03 | HK19 | CB20 |
| | (SEQ | (SEQ | (SEQ | (SEQ | (HA SEQ | (HA SEQ | (HA SEQ | (HA SEQ | (HA SEQ | (HA SEQ | (HA SEQ | (HA SEQ |
| | ID NO: | ID NO: | ID NO: | ID NO: | ID NO: | ID NO: | ID NO: | ID NO: | ID NO: | ID NO: | ID NO: | ID NO: |
| | 355) | 356) | 357) | 358) | 359) | 360) | 361) | 362) | 364) | 363) | 365) | 366) |
| CVG1B | 100 | 86 | 77 | 75 | 96 | 93 | 88 | 84 | 77 | 78 | 74 | 73 |
| (SEQ | | | | | | | | | | | | |
| ID | | | | | | | | | | | | |
| NO: | | | | | | | | | | | | |
| 355) | | | | | | | | | | | | |
| CVG2B | | 100 | 85 | 80 | 86 | 89 | 98 | 98 | 85 | 86 | 79 | 79 |
| (SEQ | | | | | | | | | | | | |
| ID | | | | | | | | | | | | |
| NO: | | | | | | | | | | | | |
| 356) | | | | | | | | | | | | |
| CVG3B | | | 100 | 93 | 76 | 77 | 83 | 84 | 96 | 98 | 90 | 91 |
| (SEQ | | | | | | | | | | | | |
| ID | | | | | | | | | | | | |
| NO: | | | | | | | | | | | | |
| 357) | | | | | | | | | | | | |
| CVG4B | | | | 100 | 74 | 75 | 79 | 79 | 92 | 92 | 95 | 95 |
| (SEQ | | | | | | | | | | | | |
| ID | | | | | | | | | | | | |
| NO: | | | | | | | | | | | | |
| 358) | | | | | | | | | | | | |

In addition to selecting clinically isolated strains that provide wide diversity across the defined H3 Component Virus Groups (FIG. 3 and FIG. 4), selected viruses included those having a higher degree of NLG (e.g., ≥3 predicted NLG sites). According to particular aspects, inclusion of NLG sites in the H3 Globular Head Region may be used to improve or tailor immunogenicity and/or immune response with the disclosed vaccines. Using the list (www.fludb.org, access date: 11 Nov. 2020) of influenza vaccine strains and strains of historical significance, predictive N-linked glycosylation analysis was performed using publicly available software (http://www.cbs.dtu.dk/services/NetNGlyc/) to compare the level of predicted NLG modifications across H3N2 strains (Table 8). The total predicted NLG varied from as few as 2, to as many as 6 predicted NLG sites across the H3 HA globular head. Particular exemplary test strains selected for vaccine development (Table 6 and Table 7) ranged from 3-6 predicted NLG per H3 HA globular head (Table 8).

TABLE 8
Predicted Influenza A H3N2 N-linked glycosylations
(NLG) in the H3 HA1 Globular Head

| Influenza | Predicted N-Linked Glycosylation Sites¹ | | | | | | | | | |
|---|---|---|---|---|---|---|---|---|---|---|
| Strain² | 45 | 63 | 81 | 126 | 133 | 144 | 158 | 165 | 246 | Total NLG |
| AI68 | | | Yes | | | | | Yes | | 2 |
| HK68 | | | Yes | | | | | Yes | | 2 |
| EN72 | | | Yes | | | | | Yes | | 2 |
| VI75 | | Yes | | Yes | | | | Yes | | 3 |
| TX77 | | Yes | | Yes | | | | Yes | | 3 |
| BK79 | | Yes | | Yes | | | | Yes | | 3 |
| SI87 | | Yes | | | | | | Yes | | 2 |
| BE89 | | Yes | | Yes | | | | Yes | Yes | 4 |
| BE92 | | Yes | | Yes | | | | Yes | Yes | 4 |
| WU95 | | Yes | | Yes | | | | Yes | Yes | 4 |
| SY97 | | Yes | | Yes | Yes | | | Yes | | 4 |
| MW99 | | Yes | | Yes | Yes | | | Yes | Yes | 5 |
| UL00 | | Yes | | Yes | Yes | | | Yes | Yes | 5 |
| FU02 | | Yes | | Yes | Yes | Yes | | Yes | Yes | 6 |
| NE03 | | Yes | | Yes | Yes | Yes | | Yes | Yes | 6 |
| CA04 | | Yes | | Yes | Yes | Yes | | Yes | Yes | 6 |
| WI05 | | Yes | | | Yes | Yes | | Yes | Yes | 5 |
| BR07[H3N2] | | Yes | | Yes | Yes | | | Yes | Yes | 5 |
| PE09 | | Yes | | Yes | Yes | | | Yes | Yes | 5 |
| VI11 | Yes | Yes | | Yes | Yes | | | Yes | Yes | 6 |
| TX12 | Yes | Yes | | | Yes | | | Yes | Yes | 5 |
| SW13 | Yes | Yes | | | Yes | Yes | | Yes | Yes | 6 |
| KS14 | Yes | Yes | | | Yes | | | Yes | Yes | 5 |
| HK14 | Yes | Yes | | Yes | Yes | | | Yes | Yes | 6 |
| SI16 | Yes | Yes | | Yes | Yes | | | Yes | Yes | 6 |
| KS17 | Yes | Yes | | | Yes | | | Yes | Yes | 5 |
| HK19 | Yes | Yes | | | | | | Yes | Yes | 4 |
| IN18 | Yes | Yes | | | Yes | | | Yes | Yes | 5 |
| MN19 | Yes | Yes | | | | | Yes | Yes | Yes | 5 |
| CB20 | Yes | Yes | | Yes | Yes | | | Yes | Yes | 6 |

¹The amino acid residue position of predicted N-linked glycosylation sites (http://www.cbs.dtu.dk/services/NetNGlyc/) and renumbered according to the H3 HA1 protein subunit. Only those potential sites that fall within the anticipated H3 HA1 globular head region are shown.
²Strains chosen as exemplary vaccine candidates are shown in bold.

### EXAMPLE 4

### Key Influenza B Antigenic Sites

Influenza B Artificial “All Antigenic Sites Combined” sequence. Similar to Influenza A H1N1 and H3N2, Influenza B also has defined antigenic sites, termed the 120 loop, the 150 loop, the 160 loop, the 190 helix and the 230 region (Skowronski, D. M., et al., supra) (Table 9). In analogy with the approach used with Influenza A, an artificial sequence referred to herein as “All Antigenic Sites Combined” (Influenza B AASC), which linearly combines/conjoins the amino acid residue locations for all five defined antigenic sites for comparison between and among Influenza B virus strains (Table 9 below).

Influenza B HA1 Globular Head (Influenza B HA1 Globular Head). As with Influenza A, an additional approach, complementary to use of the conjoined immunologically defined sites that are based on antibody binding, was to define anticipated neutralizing epitopes based on location within a larger contiguous portion of the receptor binding domain of the Influenza B HA protein. For this analysis with Influenza B, the Influenza B globular head of the HA was defined as amino acid residues 151-250 based on numbering of the Influenza B HA1 protein subunit (Table 9).

As in the above Influenza A analyses, for both Influenza B sequence analyses, some sequences were adjusted to account for upstream insertions or deletions among individual HA sequences. All sequences were adjusted to match the Influenza B HA1 numbering described in (Skowronski, D. M., et al., supra).

TABLE 9
Location of antigenic sites in the Influenza B HA1 hemagglutinin
protein¹, and location the “Influenza B Globular Head,”
referred to herein, within receptor binding domain of Influenza B HA1.

| Antigenic Site | |
|---|---|
| (n = number of | |
| amino acid residues in total) | Amino Acid Residue Number(s) |
| 120 loop (n = 29) | 73-79, 116-137 |
| 150 loop (n = 10) | 141-150 |
| 160 loop (n = 8) | 162-169 |
| 190 helix (n = 9) | 196-204 |
| 230 region (n = 16) | 228-243 |
| All Antigenic Sites | 73-79, 116-137, 141-150, |
| Combined (n = 72) | 162-169, 196-204, 228-243 |
| Influenza B HA1 Globular | 151-250 |
| Head (n = 100) | |

¹Numbering is based on the Influenza B HA1 subunit as described in (Skowronski, D. M., et al., supra).

Generating phylogenetic trees, and computing rooted phylogenetic networks from the trees. Using the same approach detailed for Influenza A above, the phylogenetic relationship between sequenced Influenza B strains was then investigated using either the Influenza B AASC amino acid sequence or the Influenza B HA1 Globular Head amino acid sequence. Available Influenza B HA protein sequences were collected from a publicly available web resource (www.fludb.org, access date: 22 Jul. 2021) and results were curated to include full HA sequences with known sample collection dates and to exclude laboratory strains. In total, 3501 HA sequences were initially analyzed. Influenza B is known to segregate into two distinct lineages, termed Victoria and Yamagata. Prior to further analysis, strains were segregated into either of these lineages, resulting in 1705 Victoria lineage strains and 1796 Yamagata lineage strains. Each lineage was then further reduced into annual consensus sequences using multiple sequence alignment (https://www.ebi.ac.uk/Tools/msa/clustalo/) followed by consensus alignment (https://www.ebi.ac.uk/Tools/msa/emboss_cons/). In cases where only one sequence was available for a calendar year, that sequence was used as the consensus sequence. For some years, no sequences were available. The sequences of particular individual vaccine strains or strains of historical significance were also included in the phylogenetic analysis. In total, 91 full-length HA protein sequences were then used to generate two data sets, one containing their respective conjoined Influenza B-AASC amino acid sequence (SEQ ID NOS: 367-457) and the other containing their respective “Influenza B-HA1 Globular Head” region (SEQ ID NOS: 460-550). Two respective phylogenetic trees were then established (https://www.ebi.ac.uk/Tools/msa/clustalo/) using these two sequence datasets (FIG. 5 and FIG. 6).

For the phylogenetic tree of FIG. 5, a total of 91 influenza B sequences, consisting of either annual consensus sequences or key historical and vaccine strains, were analyzed for their phylogenetic relatedness based on the amino acid sequence of all antigenic sites combined (Influenza B-AASC) as described in the methods. Prior to consensus building, strains were first segregated into Victoria-like or Yamagata-like sequences. Annual consensus sequences may include years where multiple sequences were available (i.e., Vic-Year-cons or Yam-Year-cons) or years where only one sequence was available (i.e., Vic-Year-single or Yam-Year-single). Historical/vaccine strains included the following: B/Lee/1940, Vic_LE40; B/Victoria/02/1987, Victoria_1987; B/Oregon/5/80, Vic_OR80; B/Ann Arbor/1/1986, Vic_AA86; B/Hong Kong/330/2001, Vic_HK01; B/New_York/1055/2003, Vic_NY03; B/Malaysia/2506/2004, Vic_ML04; B/Ohio/01/2005, Vic_OH05; B/Brisbane/60/2008, Vic_BR08; B/Nevada/03/2011, Vic_NV11; B/Colorado/06/2017, Vic_CO17; B/Yamagata/16/1988, Yamagata_1988; B/Panama/45/1990, Yam_PA90; B/Harbin/7/1994, Yam_HA94; B/Christchurch/33/2004, Yam_CC04; B/New_York/1061/2004, Yam_NY04; B/Florida/4/2006, Yam_FL06; B/Sydney/507/2006, Yam_SY06; B/Texas/06/2011, Yam_TX11; B/Phuket/3073/2013, Yam_PH13. Component virus groupings were delineated as shown, and two vaccine strains within each grouping were selected for evaluation as vaccine candidates (bolded). For all historical and vaccine strains, the level of predicted N-linked glycosylations within the HA1 globular head was calculated and is shown next to each strain.

For the phylogenetic tree of FIG. 6, a total of 91 influenza B sequences, consisting of either annual consensus sequences or key historical and vaccine strains, were analyzed for their phylogenetic relatedness based on the HA1 globular head as described in the methods. Prior to consensus building, strains were first segregated into Victoria-like or Yamagata-like sequences. Annual consensus sequences may include years where multiple sequences were available (i.e. Vic-Year-cons or Yam-Year-cons) or years where only one sequence was available (i.e., Vic-Year-single or Yam-Year-single). Historical/vaccine strains included the following: B/Lee/1940, Vic_LE40; B/Victoria/02/1987, Victoria_1987; B/Oregon/5/80, Vic_OR80; B/Ann_Arbor/1/1986, Vic_AA86; B/Hong_Kong/330/2001, Vic_HK01; B/New_York/1055/2003, Vic_NY03; B/Malaysia/2506/2004, Vic_ML04; B/Ohio/01/2005, Vic_OH05; B/Brisbane/60/2008, Vic_BR08; B/Nevada/03/2011, Vic_NV11; B/Colorado/06/2017, Vic_CO17; B/Yamagata/16/1988, Yamagata_1988; B/Panama/45/1990, Yam_PA90; B/Harbin/7/1994, Yam_HA94; B/Christchurch/33/2004, Yam_CC04; B/New_York/1061/2004, Yam_NY04; B/Florida/4/2006, Yam_FL06; B/Sydney/507/2006, Yam_SY06; B/Texas/06/2011, Yam_TX11; B/Phuket/3073/2013, Yam_PH13. Component virus groupings were delineated as shown, and two vaccine strains within each grouping were selected for evaluation as vaccine candidates (bolded). For all historical and vaccine strains, the level of predicted N-linked glycosylations within the HA1 globular head was calculated and is shown next to each strain.

The phylogenetic trees were further analyzed by computing rooted phylogenetic networks from the trees (Dendroscope v3.7.5) and visualized as rectangular phylograms with midpoint roots. Following this analysis, two distinct groupings were identified in both phylogenetic trees (FIG. 5 and FIG. 6). For the “Influenza B-AASC” tree/network, these were defined as Influenza B Component Virus Groups 1A and 2A (Influenza B-CVG1A and Influenza B-CVG2A), and for the “Influenza B HA1 Globular Head” tree/network as analogous Influenza B Component Virus Groups 1B and 2B (Influenza B-CVG1B and Influenza B-CVG 2B), where the viral strains encompassed by analogous groups (e.g., by Influenza B-CVG1A and Influenza B-CVG1B, etc.) were substantially the same.

Using these two clusters as a guide, two exemplary virus strains were selected from each of the two Influenza B-AASC component groups (Influenza B-CVG1A and Influenza B-CVG2A), and from each of the two “Influenza B Globular Head” groups (Influenza B-CVG1B and Influenza B-CVG2B), to test as potential vaccine candidates. The selected strains were: B/Malaysia/2506/2004 (Vic_ML04) (having full HA SEQ ID NO:553), B/Nevada/03/2011 (Vic_NV11) (having full HA SEQ ID NO:554) (both encompassed by either Influenza B-CVG1A or Influenza B-CVG1B); B/Texas/06/2011 (Yam_TX11) (having full HA SEQ ID NO:555) and B/Phuket/3073/2013 (Yam_PH13) (having full HA SEQ ID NO: 556) (both encompassed by either Influenza B-CVG2A or Influenza B-CVG2B).

The exemplary diverse test strains were clinically isolated influenza strains that could be developed into vaccine candidates and provided a breadth of sequence diversity within the disclosed individual component virus groups (Influenza B-CVG1A and Influenza B-CVG2A, and Influenza B-CVG1B and Influenza B-CVG2B). Respective consensus sequences were also determined for each of the Influenza B-CVG1A and Influenza B-CVG2A (Influenza B-AASC) groups (SEQ ID NOS: 458 and 459, respectively), and for each of the Influenza B-CVG1B and Influenza B-CVG2B) (“Influenza B-HA1 Globular Head”) groups (SEQ ID NOS: 551 and 552, respectively), in each case based on all the viral Influenza B HA sequences used to define the respective groupings.

Sequence comparisons (% identity) were then made between the Influenza B-AASC and Influenza B-HA1 Globular Head sequences of each test strain and the consensus sequences of their respective groupings; Influenza B-CVG1A and Influenza B-CVG2A (Influenza B-AASC) groups, and the Influenza B-CVG1B and Influenza B-CVG2B (“Influenza B-HA1 Globular Head”) groups, as shown below in Table 10 (Influenza B-AASC comparison) and Table 11 (“Influenza B-HA1 Globular Head” comparison), respectively.

TABLE 10
Sequence comparison (% identity) based on all antigenic
sites combined (Influenza B-AASC) sequences.

| | | | Select Virus Strains | | | |
|---|---|---|---|---|---|---|
| | Component Virus Groups | | Vic_ML04 | Vic_NV11 | Yam_TX11 | Yam_PH13 |
| | CVG1A | CVG2A | (HA SEQ | (HA SEQ | (HA SEQ | (HA SEQ |
| | (SEQ ID | (SEQ ID | ID NO: | ID NO: | ID NO: | ID NO: |
| | NO: 458) | NO: 459) | 553) | 554) | 555) | 556) |
| CVG1A | 100 | 75 | 94 | 96 | 74 | 74 |
| (SEQ ID | | | | | | |
| NO: 458) | | | | | | |
| CVG2A | | 100 | 74 | 75 | 97 | 94 |
| (SEQ ID | | | | | | |
| NO: 459) | | | | | | |

TABLE 11
Sequence Comparison (% identity) Based on “Influenza
B HA1 Globular Head” sequences.

| | | | Select Virus Strains | | | |
|---|---|---|---|---|---|---|
| | Component Virus Groups | | Vic_ML04 | Vic_NV11 | Yam_TX11 | Yam_PH13 |
| | CVG1B | CVG2B | (HA SEQ | (HA SEQ | (HA SEQ | (HA SEQ |
| | (SEQ ID | (SEQ ID | ID NO: | ID NO: | ID NO: | ID NO: |
| | NO: 551) | NO: 552) | 553) | 554) | 555) | 556) |
| CVG1B | 100 | 89 | 98 | 98 | 86 | 88 |
| (SEQ ID | | | | | | |
| NO: 551) | | | | | | |
| CVG2B | | 100 | 88 | 89 | 96 | 97 |
| (SEQ ID | | | | | | |
| NO: 552) | | | | | | |

N-linked glycosylation (NLG) sites. In addition to selecting clinically isolated strains that provide wide diversity across the defined Influenza B Component Virus Groups (FIG. 5 and FIG. 6) the selected viruses included those having a higher degree of NLG (e.g., ≥5 predicted NLG sites). According to particular aspects, inclusion of NLG sites in the Influenza B Globular Head Region may be used to improve or tailor immunogenicity and/or immune response with the disclosed vaccines. Using the list (www.fludb.org, access date: 22 Jul. 2021) of influenza vaccine strains and strains of historical significance, predictive N-linked glycosylation analysis was performed using publicly available software (http://www.cbs.dtu.dk/services/NetNGlyc/) to compare the level of predicted NLG modifications across strains (Table 12). The total predicted NLG varied from as few as 3, to as many as 6 predicted NLG sites across the Influenza B-HA1 Globular Head. The exemplary test strains selected for vaccine development (Table 10 and Table 11) ranged from 5-6 predicted NLG per Influenza B-HA1 globular head (Table 12).

TABLE 12
Predicted Influenza B N-linked glycosylation (NLG)
sites in the Influenza B-HA1 globular head

| | Predicted N-Linked Glycosylation Sites¹ | | | | | | |
|---|---|---|---|---|---|---|---|
| Influenza | | | | | | | Total |
| Strain² | 145 | 165 | 197 | 233 | 304 | 333 | NLG |
| Victoria_1987 | Yes | Yes | | | Yes | Yes | 4 |
| Vic_LE40 | | Yes | | | Yes | Yes | 3 |
| Vic_OR80 | Yes | Yes | | | Yes | Yes | 4 |
| Vic_AA86 | Yes | Yes | | | Yes | Yes | 4 |
| Vic_HK01 | Yes | Yes | | Yes | Yes | Yes | 5 |
| Vic_NY03 | Yes | Yes | Yes | Yes | Yes | Yes | 6 |
| Vic_{—}ML04 | Yes | Yes | | Yes | Yes | Yes | 5 |
| Vic_OH05 | Yes | Yes | | Yes | Yes | Yes | 5 |
| Vic_BR08 | Yes | Yes | | Yes | Yes | Yes | 5 |
| Vic_{—}NV11 | Yes | Yes | Yes | Yes | Yes | Yes | 6 |
| Vic_CO17 | Yes | Yes | | Yes | Yes | Yes | 5 |
| Yamagata_1988 | Yes | Yes | | | Yes | Yes | 4 |
| Yam_PA90 | Yes | Yes | | Yes | Yes | Yes | 5 |
| Yam_HA94 | Yes | Yes | | | Yes | Yes | 4 |
| Yam_CC04 | Yes | Yes | Yes | | Yes | Yes | 5 |
| Yam_NY04 | Yes | Yes | Yes | | Yes | Yes | 5 |
| Yam_FL06 | Yes | Yes | | | Yes | Yes | 4 |
| Yam_SY06 | Yes | Yes | Yes | | Yes | Yes | 5 |
| Yam_{—}TX11 | Yes | Yes | Yes | | Yes | Yes | 5 |
| Yam_{—}PH13 | Yes | Yes | Yes | | Yes | Yes | 5 |

¹The amino acid residue position of predicted N-linked glycosylation sites (http://www.cbs.dtu.dk/services/NetNGlyc/) and renumbered according to the Influenza B-HA1 protein subunit. Only those potential sites that fall within the anticipated globular head region are shown.
²Strains chosen as exemplary vaccine candidates are shown in bold.

TABLE 13
Summary of SEQ ID NOS:

| Sequence | |
|---|---|
| Number | Description |
| 1-84 | H1N1 All Antigenic Site Sequences |
| 85 | H1N1 CVG1A Consensus |
| 86 | H1N1 CVG2A Consensus |
| 87 | H1N1 CVG3A Consensus |
| 88 | H1N1 CVG4A Consensus |
| 89-172 | H1N1 HA Globular Head Sequences |
| 173 | H1N1 CVG1B Consensus |
| 174 | H1N1 CVG2B Consensus |
| 175 | H1N1 CVG3B Consensus |
| 176 | H1N1 CVG4B Consensus |
| 177 | A/WSN/1933 |
| 178 | A/PR/8/1934 |
| 179 | A/Fort Monmouth/1/1947 |
| 180 | A/USSR/90/1977 |
| 181 | A/Solomon Islands/3/2006 |
| 182 | A/Brisbane/59/2007 |
| 183 | A/Michigan/45/2015 |
| 184 | A/Nebraska/14/2019 |
| 185-267 | H3N2 All Antigenic Site Sequences |
| 268 | H3N2 CVG1A Consensus |
| 269 | H3N2 CVG2A Consensus |
| 270 | H3N2 CVG3A Consensus |
| 271 | H3N2 CVG4A Consensus |
| 272-354 | H3N2 HA Globular Head Sequences |
| 355 | H3N2 CVG1B Consensus |
| 356 | H3N2 CVG2B Consensus |
| 357 | H3N2 CVG3B Consensus |
| 358 | H3N2 CVG4B Consensus |
| 359 | A/Texas/1/1977 full HA |
| 360 | A/Bangkok/1/1979 full HA |
| 361 | A/Beijing/353/1989 full HA |
| 362 | A/Beijing/32/1992 full HA |
| 363 | A/Netherlands/22/2003 full HA |
| 364 | A/Fujian/411/2002 full HA |
| 365 | A/Hong Kong/2671/2019 full HA |
| 366 | A/Cambodia/e0826360/2020 full HA |
| 367-457 | Influenza B All Antigenic Site Sequences |
| 458 | Influenza B CVG1A Consensus |
| 459 | Influenza B CVG2A Consensus |
| 460-550 | Influenza B HA Globular Head Sequences |
| 551 | Influenza B CVG1B Consensus |
| 552 | Influenza B CVG2B Consensus |
| 553 | B/Malaysia/2506/2004 |
| 554 | B/Nevada/03/2011 |
| 555 | B/Texas/06/2011 |
| 556 | B/Phuket/3073/2013 |

References cited, and incorporated herein by reference for their respective teachings:
1. Thompson, W. W., et al., Influenza-associated hospitalizations in the United States. JAMA, 2004. 292 (11): p. 1333-40.
2. CDC, Estimates of deaths associated with seasonal influenza—United States, 1976-2007. MMWR Morb Mortal Wkly Rep, 2010. 59 (33): p. 1057-62.
3. Matias, G., et al., Estimates of mortality attributable to influenza and RSV in the United States during 1997-2009 by influenza type or subtype, age, cause of death, and risk status. Influenza Other Respir Viruses, 2014. 8 (5): p. 507-15.
4. Osterholm, M. T., et al., Efficacy and effectiveness of influenza vaccines: a systematic review and meta-analysis. Lancet Infect Dis, 2012. 12 (1): p. 36-44.
5. Flannery, B., et al., Interim Estimates of 2017-18 Seasonal Influenza Vaccine Effectiveness—United States, February 2018. MMWR Morb Mortal Wkly Rep, 2018. 67 (6): p. 180-185.
6. Young, B., et al., Do antibody responses to the influenza vaccine persist year-round in the elderly? A systematic review and meta-analysis. Vaccine, 2017. 35 (2): p. 212-221.
7. Radin, J. M., et al., Influenza vaccine effectiveness: Maintained protection throughout the duration of influenza seasons 2010-2011 through 2013-2014. Vaccine, 2016. 34 (33): p. 3907-12.
8. Ferdinands, J. M., et al., Intraseason waning of influenza vaccine protection: Evidence from the US Influenza Vaccine Effectiveness Network, 2011-12 through 2014-15. Clin Infect Dis, 2017. 64 (5): p. 544-550.
9. Jimenez-Jorge, S., et al., Effectiveness of influenza vaccine against laboratory-confirmed influenza, in the late 2011-2012 season in Spain, among population targeted for vaccination. BMC Infect Dis, 2013. 13: p. 441.
10. Gerhard, W., et al., Antigenic structure of influenza virus haemagglutinin defined by hybridoma antibodies. Nature, 1981. 290 (5808): p. 713-7.
11. Caton, A. J., et al., The antigenic structure of the influenza virus A/PR/8/34 hemagglutinin (H1 subtype). Cell, 1982. 31 (2 Pt 1): p. 417-27.
12. Brownlee, G. G. and E. Fodor, The predicted antigenicity of the haemagglutinin of the 1918 Spanish influenza pandemic suggests an avian origin. Philos Trans R Soc Lond B Biol Sci, 2001. 356 (1416): p. 1871-6.
13. Skowronski, D. M., et al., Low 2012-13 influenza vaccine effectiveness associated with mutation in the egg-adapted H3N2 vaccine strain not antigenic drift in circulating viruses. PLOS One, 2014. 9 (3): p. e92153.
14. Sun, S., et al., Glycosylation site alteration in the evolution of influenza A(H1N1) viruses. PLOS One, 2011. 6 (7): p. e22844.
15. Skowronski, D. M., et al., Integrated Sentinel Surveillance Linking Genetic, Antigenic, and Epidemiologic Monitoring of Influenza Vaccine-Virus Relatedness and Effectiveness During the 2013-2014 Influenza Season. J Infect Dis, 2015. 212 (5): p. 726-39.
16. Quintel, B. K., et al., Advanced oxidation technology for the development of a next-generation inactivated West Nile virus vaccine. Vaccine, 2019. 37 (30): p. 4214-4221.
17. World Health Organization., Manual for the laboratory diagnosis and virological surveillance of influenza. 2011, Geneva: World Health Organization. xii, 139 p.
18. Hammarlund, E., et al., A flow cytometry-based assay for quantifying non-plaque forming strains of yellow fever virus. PLoS One, 2012. 7 (9): p. e41707.

What is claimed is:

1. A multivalent pan-influenza vaccine, comprising a viral haemagglutinin (HA) protein or HA1-containing portion thereof, and/or comprising a nucleic acid encoding the HA protein or the HA1-containing portion thereof, of or corresponding to a virus strain from each of any three of, or from all four of component virus strain groups H1-CVG1-H1-CVG-4, wherein:
H1-CVG-1 comprises H1N1 virus strains having either (i) a conjoined Sa, Sb, Ca1, Ca2, and Cb HA antigenic sites amino acid (aa) sequence having at least 82% sequence identity with SEQ ID NO:85, and/or (ii) a HA1 Globular Head Region aa sequence having at least 91% sequence identity with SEQ ID NO:173;
H1-CVG-2 comprises H1N1 virus strains having either (i) a conjoined Sa, Sb, Ca1, Ca2, and Cb HA antigenic sites aa sequence having at least 90% sequence identity with SEQ ID NO: 86, and/or (ii) a HA1 Globular Head Region aa sequence having at least 96% sequence identity with SEQ ID NO:174;
H1-CVG-3 comprises H1N1 virus strains having either (i) a conjoined Sa, Sb, Ca1, Ca2, and Cb HA antigenic sites aa sequence having at least 92% sequence identity with SEQ ID NO: 87, and/or (ii) a HA1 Globular Head Region aa sequence having at least 93% sequence identity with SEQ ID NO: 175; and
H1-CVG-4 comprises H1N1 virus strains having either (i) a conjoined Sa, Sb, Ca1, Ca2, and Cb HA antigenic sites aa sequence having at least 88% sequence identity with SEQ ID NO: 88, and/or (ii) a HA1 Globular Head Region aa sequence having at least 96% sequence identity with SEQ ID NO:176.

2. The vaccine of claim 1, wherein:
the virus strain from H1-CVG-1 is WS33 having HA SEQ ID NO: 177, and/or is PR8 having HA SEQ ID NO: 178; and/or
the virus strain from H1-CVG-2 is FM47 having HA SEQ ID NO:179, and/or is USSR77 having HA SEQ ID NO: 180; and/or
the virus strain from H1-CVG-3 is BR07 having HA SEQ ID NO: 182, and/or is SI06 having HA SEQ ID NO: 181; and/or
the virus strain from H1-CVG-4 is NEB19 having HA SEQ ID NO:184, and/or is MCH15 having HA SEQ ID NO: 183.

3. The vaccine of claim 2, wherein:
the virus strain from H1-CVG-1 is WS33 having HA SEQ ID NO:177, and/or is PR8 having HA SEQ ID NO:178; and
the virus strain from H1-CVG-2 is FM47 having HA SEQ ID NO:179, and/or is USSR77 having HA SEQ ID NO:180; and
the virus strain from H1-CVG-3 is BR07 having HA SEQ ID NO:182, and/or is SI06 having HA SEQ ID NO:181; and
the virus strain from H1-CVG-4 is NEB19 having HA SEQ ID NO:184, and/or is MCH15 having HA SEQ ID NO: 183.

4. The vaccine of claim 2, wherein:
the virus strain from H1-CVG-1 is WS33; and/or
the virus strain from H1-CVG-2 is FM47; and/or
the virus strain from H1-CVG-is BR07; and/or
the virus strain from H1-CVG-4 is NEB19.

5. The vaccine of claim 4, wherein:
the virus strain from H1-CVG-1 is WS33; and
the virus strain from H1-CVG-2 is FM47; and
the virus strain from H1-CVG-3 is BR07; and
the virus strain from H1-CVG-4 is NEB19.

6. The vaccine of any one of claims 1-5, wherein:
the Globular Head Region of the virus strain from H1-CVG-1 comprises one or more predicted and/or confirmed N-linked glycosylation site(s) (NLGs); and/or
the Globular Head Region of the virus strain from H1-CVG-2 comprises one or more predicted and/or confirmed NLGs; and/or
the Globular Head Region of the virus strain from H1-CVG-3 comprises one or more predicted and/or confirmed NLGs; and/or
the Globular Head Region of the virus strain from H1-CVG-4 comprises one or more predicted and/or confirmed NLGs (NLGs).

7. The vaccine of claim 6, wherein:
the Globular Head Region of the virus strain from H1-CVG-1 comprises one or more predicted and/or confirmed NLGs; and
the Globular Head Region of the virus strain from H1-CVG-2 comprises one or more predicted and/or confirmed NLGs; and
the Globular Head Region of the virus strain from H1-CVG-3 comprises one or more predicted and/or confirmed NLGs; and
the Globular Head Region of the virus strain from H1-CVG-4 comprises one or more predicted and/or confirmed NLGs (NLGs).

8. The vaccine of claim 6, wherein:
the Globular Head Region of the virus strain from H1-CVG-1 comprises at least two predicted and/or confirmed NLGs; and/or
the Globular Head Region of the virus strain from H1-CVG-2 comprises at least three predicted and/or confirmed NLGs; and/or
the Globular Head Region of the virus strain from H1-CVG-3 comprises at least four predicted and/or confirmed NLGs; and/or
the Globular Head Region of the virus strain from H1-CVG-4 comprises at least two predicted and/or confirmed NLGs.

9. The vaccine of claim 8, wherein:
the Globular Head Region of the virus strain from H1-CVG-1 comprises at least two predicted and/or confirmed NLGs; and
the Globular Head Region of the virus strain from H1-CVG-2 comprises at least three predicted and/or confirmed NLGs; and
the Globular Head Region of the virus strain from H1-CVG-3 comprises at least four predicted and/or confirmed NLGs; and
the Globular Head Region of the virus strain from H1-CVG-4 comprises at least two predicted and/or confirmed NLGs.

10. The vaccine of any one of claims 1-9, wherein the HA proteins or HA1-containing portions thereof from the any three of, or the four component virus groups are present as one or more components that can be administered together, or sequentially.

11. The vaccine of claim 10, wherein the HA proteins or HA1-containing portions thereof from the any three of, or the four component virus groups are combined in a multivalent vaccine composition for coadministration.

12. The vaccine of any one of claims 1-11, further comprising an adjuvant, and/or a pharmaceutically acceptable carrier, diluent, or excipient.

13. The vaccine of claim 12, wherein the adjuvant comprises one or more aluminum salts.

14. The vaccine of any one of claims 1-13, wherein, independently with respect to each of the any three of, or the four component virus groups, the HA protein(s) or the HA1-containing portion(s) thereof is present as one or more of: a component of an inactivated virus or component thereof; a component of a recombinant virus or component thereof; a recombinant HA or component thereof; and/or a component of a nanoparticle vaccine delivery platform/composition.

15. The vaccine of claim 14, wherein, independently with respect to each of the any three of, or the four component virus groups, the HA protein(s) or the HA1-containing portion(s) thereof is present as one or more of: a component of an inactivated virus or component thereof; and/or as a recombinant HA or component thereof.

16. The vaccine of claim 15, wherein, with respect to the any three of, or the four component virus groups, the HA protein(s) or the HA1-containing portion(s) thereof is present as a component of an inactivated virus or component thereof, or as a recombinant HA or component thereof.

17. The vaccine of claim 16, wherein, with respect to the any three of, or the four component virus groups, the HA protein(s) or the HA1-containing portion(s) thereof is present as a component of an inactivated virus or component thereof.

18. The vaccine of any one of claims 1-17, wherein for each of the any three of, or the four component virus groups, the vaccine comprises the HA protein(s), or the HA1-containing portion(s) thereof, of only one viral strain per group.

19. A method of eliciting an immune response, comprising administering an immunogenic vaccine composition according to any one of claims 1-18 to a subject, thereby eliciting in the subject an immune response against influenza.

20. The method of claim 19, wherein eliciting the immune response comprises eliciting an H1N1 influenza virus-specific immune response, and/or a pan-H1N1 influenza virus-specific immune response.

21. The method of claim 20, wherein eliciting the immune response additionally comprises eliciting an immune response to at least one non-H1N1 vaccine strain.

22. The method of any one of claims 19-21, wherein the immune response comprises one or more of an antibody, a B cell, and/or a T cell response.

23. The method of any one of claims 19-22, wherein administration comprises administering the vaccine in one or more components administered together, or sequentially.

24. A multivalent pan-influenza vaccine, comprising a viral haemagglutinin (HA) protein, or HA1-containing portion thereof, and/or comprising a nucleic acid encoding the HA protein or the HA1-containing portion thereof, of or corresponding to a virus strain from each of any three of, or from all four of component virus strain groups H3-CVG-1-H3-CVG-4, wherein:
H3-CVG-1 comprises H3N2 virus strains having either (i) a conjoined A, B, C, D, and E HA antigenic sites amino acid (aa) sequence having at least 88% sequence identity with SEQ ID NO:268, and/or (ii) a HA1 Globular Head Region aa sequence having at least 93% sequence identity with SEQ ID NO:355;
H3-CVG-2 comprises H3N2 virus strains having either (i) a conjoined A, B, C, D, and E HA antigenic sites aa sequence having at least 95% sequence identity with SEQ ID NO: 269, and/or (ii) a HA1 Globular Head Region aa sequence having at least 98% sequence identity with SEQ ID NO:356;
H3-CVG-3 comprises H3N2 virus strains having either (i) a conjoined A, B, C, D, and E HA antigenic sites aa sequence having at least 93% sequence identity with SEQ ID NO: 270, and/or (ii) a HA1 Globular Head Region aa sequence having at least 96% sequence identity with SEQ ID NO:357; and
H3-CVG-4 comprises H3N2 virus strains having either (i) a conjoined A, B, C, D, and E HA antigenic sites aa sequence having at least 89% sequence identity with SEQ ID NO: 271, and/or (ii) a HA1 Globular Head Region aa sequence having at least 95% sequence identity with SEQ ID NO:358.

25. The vaccine of claim 24, wherein:
the virus strain from H3-CVG-1 is TX77 having HA SEQ ID NO:359, and/or is BK79 having HA SEQ ID NO:360; and/or
the virus strain from H3-CVG-2 is BE89 having HA SEQ ID NO:361, and/or is BE92 having HA SEQ ID NO:362; and/or
the virus strain from H3-CVG-3 is FU02 having HA SEQ ID NO:364, and/or is NE03 having HA SEQ ID NO:363; and/or
the virus strain from H3-CVG-4 is HK19 having HA SEQ ID NO:365, and/or is CB20 having HA SEQ ID NO:366.

26. The vaccine of claim 25, wherein:
the virus strain from H3-CVG-1 is TX77 having HA SEQ ID NO:359, and/or is BK79 having HA SEQ ID NO:360; and
the virus strain from H3-CVG-2 is BE89 having HA SEQ ID NO:361, and/or is BE92 having HA SEQ ID NO:362; and
the virus strain from H3-CVG-3 is FU02 having HA SEQ ID NO:364, and/or is NE03 having HA SEQ ID NO:363; and
the virus strain from H3-CVG-4 is HK19 having HA SEQ ID NO:365, and/or is CB20 having HA SEQ ID NO:366.

27. The vaccine of claim 25, wherein:
the virus strain from H3-CVG-1 is TX77; and/or
the virus strain from H3-CVG-2 is BE89; and/or
the virus strain from H3-CVG-is FU02; and/or
the virus strain from H3-CVG-4 is HK19

28. The vaccine of claim 27, wherein:
the virus strain from H3-CVG-1 is TX77; and
the virus strain from H3-CVG-2 is BE89; and
the virus strain from H3-CVG-3 is FU02; and
the virus strain from H3-CVG-4 is HK19.

29. The vaccine of any one of claims 24-28, wherein:
the Globular Head Region of the virus strain from H3-CVG-1 comprises one or more predicted and/or confirmed N-linked glycosylation site(s) (NLGs); and/or
the Globular Head Region of the virus strain from H3-CVG-2 comprises one or more predicted and/or confirmed NLGs; and/or
the Globular Head Region of the virus strain from H3-CVG-3 comprises one or more predicted and/or confirmed NLGs; and/or
the Globular Head Region of the virus strain from H3-CVG-4 comprises one or more predicted and/or confirmed NLGs (NLGs).

30. The vaccine of claim 29, wherein:
the Globular Head Region of the virus strain from H3-CVG-1 comprises one or more predicted and/or confirmed NLGs; and
the Globular Head Region of the virus strain from H3-CVG-2 comprises one or more predicted and/or confirmed NLGs; and
the Globular Head Region of the virus strain from H3-CVG-3 comprises one or more predicted and/or confirmed NLGs; and
the Globular Head Region of the virus strain from H3-CVG-4 comprises one or more predicted and/or confirmed NLGs (NLGs).

31. The vaccine of claim 29, wherein:
the Globular Head Region of the virus strain from H3-CVG-1 comprises at least three predicted and/or confirmed NLGs; and/or
the Globular Head Region of the virus strain from H3-CVG-2 comprises at least four predicted and/or confirmed NLGs; and/or
the Globular Head Region of the virus strain from H3-CVG-3 comprises at least six predicted and/or confirmed NLGs; and/or
the Globular Head Region of the virus strain from H3-CVG-4 comprises at least four predicted and/or confirmed NLGs.

32. The vaccine of claim 31, wherein:
the Globular Head Region of the virus strain from H3-CVG-1 comprises at least three predicted and/or confirmed NLGs; and
the Globular Head Region of the virus strain from H3-CVG-2 comprises at least four predicted and/or confirmed NLGs; and
the Globular Head Region of the virus strain from H3-CVG-3 comprises at least six predicted and/or confirmed NLGs; and
the Globular Head Region of the virus strain from H3-CVG-4 comprises at least four predicted and/or confirmed NLGs.

33. The vaccine of any one of claims 24-32, wherein the HA proteins or HA1-containing portions thereof from the any three of, or the four component virus groups are present in one or more components that can be administered together, or sequentially.

34. The vaccine of claim 33, wherein the HA proteins or HA1-containing portions thereof from the any three of, or the four component virus groups are combined in a multivalent vaccine composition for coadministration.

35. The vaccine of any one of claims 24-34, further comprising an adjuvant, and/or a pharmaceutically acceptable carrier, diluent, or excipient. 36 The vaccine of claim 35, wherein the adjuvant comprises one or more aluminum salts.

37. The vaccine of any one of claims 24-36, wherein, independently with respect to each of the any three of, or the four component virus groups, the HA protein(s) or the HA1-containing portion(s) thereof is present as one or more of: a component of an inactivated virus component thereof; a component of a recombinant virus or component thereof; a recombinant HA or component thereof; and/or a component of a nanoparticle vaccine delivery platform/composition.

38. The vaccine of claim 37, wherein, independently with respect to each of the any three of, or the four component virus groups, the HA protein(s) or the HA1-containing portion(s) thereof is present as one or more of: a component of an inactivated virus or component thereof; and/or as a recombinant HA or component thereof.

39. The vaccine of claim 38, wherein, with respect to the any three of, or the four component virus groups, the HA protein(s) or the HA1-containing portion(s) thereof is present as a component of an inactivated virus or component thereof, or as a recombinant HA or component thereof.

40. The vaccine of claim 39, wherein, with respect to the any three of, or the four component virus groups, the HA protein(s) or the HA1-containing portion(s) thereof is present as a component of an inactivated virus or component thereof.

41. The vaccine of any one of claims 24-40, wherein for each of the any three of, or the four component virus groups, the vaccine comprises the HA protein(s), or the HA1-containing portion(s) thereof, of only one viral strain for each group.

42. A method of eliciting an immune response, comprising administering an immunogenic vaccine composition according to any one of claims 24-41 to a subject, thereby eliciting in the subject an immune response against influenza.

43. The method of claim 42, wherein eliciting the immune response comprises eliciting an H3N2 influenza virus-specific immune response, and/or a pan-H3N2 influenza virus-specific immune response.

44. The method of claim 43, wherein eliciting the immune response additionally comprises eliciting an immune response to at least one non-H3N2 vaccine strain.

45. The method of any one of claims 42-44, wherein the immune response comprises one or more of an antibody, a B cell, and/or a T cell response.

46. The method of any one of claims 24-45, wherein administration comprises administering the vaccine in one or more components administered together, or sequentially.

47. A multivalent pan-influenza vaccine, comprising a viral haemagglutinin (HA) protein, or HA1-containing portion thereof, and/or comprising a nucleic acid encoding the HA protein or the HA1-containing portion thereof, of or corresponding to a virus strain from each of two component virus strain groups Influenza B-CVG-1 and Influenza B-CVG-2, wherein:
Influenza B-CVG-1 comprises Influenza B virus strains having either (i) a conjoined 120 loop, 150 loop, 160 loop, 190 helix, and 230 region HA antigenic sites amino acid (aa) sequence having at least 94% sequence identity with SEQ ID NO:458, and/or (ii) a HA1 Globular Head Region aa sequence having at least 98% sequence identity with SEQ ID NO: 551; and
Influenza B-CVG-2 comprises Influenza B virus strains having either (i) a conjoined 120 loop, 150 loop, 160 loop, 190 helix, and 230 region HA antigenic sites aa sequence having at least 94% sequence identity with SEQ ID NO:459, and/or (ii) a HA1 Globular Head Region aa sequence having at least 96% sequence identity with SEQ ID NO:552.

48. The vaccine of claim 47, wherein:
the virus strain from Influenza B-CVG-1 is Vic_ML04 having HA SEQ ID NO:553, and/or is Vic_NV11 having HA SEQ ID NO:554; and/or
the virus strain from Influenza B-CVG-2 is Yam_TX11 having HA SEQ ID NO:555, and/or is Yam_PH13 having HA SEQ ID NO:556.

49. The vaccine of claim 48, wherein:
the virus strain from Influenza B-CVG-1 is Vic_ML04 having HA SEQ ID NO:553, and/or is Vic_NV11 having HA SEQ ID NO:554; and
the virus strain from Influenza B-CVG-2 is Yam_TX11 having HA SEQ ID NO:555, and/or is Yam_PH13 having HA SEQ ID NO:556.

50. The vaccine of claim 48, wherein:
the virus strain from Influenza B-CVG-1 is Vic_ML04; and/or
the virus strain from Influenza B-CVG-2 is Yam_TX11.

51. The vaccine of claim 50, wherein:
the virus strain from Influenza B-CVG-1 is Vic_ML04; and
the virus strain from Influenza B-CVG-2 is Yam_TX11.

52. The vaccine of any one of claims 47-51, wherein:
the Globular Head Region of the virus strain from Influenza B-CVG-1 comprises one or more predicted and/or confirmed N-linked glycosylation site(s) (NLGs); and/or
the Globular Head Region of the virus strain from Influenza B-CVG-2 comprises one or more predicted and/or confirmed NLGs.

53. The vaccine of claim 52, wherein:
the Globular Head Region of the virus strain from Influenza B-CVG-1 comprises one or more predicted and/or confirmed NLGs; and
the Globular Head Region of the virus strain from Influenza B-CVG-2 comprises one or more predicted and/or confirmed NLGs.

54. The vaccine of claim 52, wherein:
the Globular Head Region of the virus strain from Influenza B-CVG-1 comprises at least five predicted and/or confirmed NLGs; and/or
the Globular Head Region of the virus strain from Influenza B-CVG-2 comprises at least five predicted and/or confirmed NLGs.

55. The vaccine of claim 54, wherein:
the Globular Head Region of the virus strain from Influenza B-CVG-1 comprises at least five predicted and/or confirmed NLGs; and
the Globular Head Region of the virus strain from Influenza B-CVG-2 comprises at least five predicted and/or confirmed NLGs.

56. The vaccine of any one of claims 47-55, wherein the HA protein or HA1-containing portion thereof from each of the two component virus groups are present as one or more components that can be administered together, or sequentially.

57. The vaccine of claim 56, wherein the HA protein or HA1-containing portion thereof from each of the two component virus groups are combined in a multivalent vaccine composition for coadministration.

58. The vaccine of any one of claims 47-57, further comprising an adjuvant, and/or a pharmaceutically acceptable carrier, diluent, or excipient.

59. The vaccine of claim 58, wherein the adjuvant comprises one or more aluminum salts

60. The vaccine of any one of claims 47-59, wherein, independently with respect to each of the two component virus groups, the HA protein(s) or the HA1-containing portion(s) thereof is present as one or more of: a component of an inactivated virus or component thereof; a component of a recombinant virus or component thereof; recombinant HA or component thereof; and/or a component of a nanoparticle vaccine delivery platform/composition.

61. The vaccine of claim 60, wherein, independently with respect to each of the two component virus groups, the HA protein(s) or the HA1-containing portion(s) thereof is present as one or more of: a component of an inactivated virus or component thereof; and/or as a recombinant HA or component thereof.

62. The vaccine of claim 61, wherein, with respect to both component virus groups, the HA protein(s) or the HA1-containing portion(s) thereof is present as a component of an inactivated virus or component thereof, or as a recombinant HA or component thereof.

63. The vaccine of claim 62, wherein, with respect to both component virus groups, the HA protein(s) or the HA1-containing portion(s) thereof is present as a component of an inactivated virus or component thereof.

64. The vaccine of any one of claims 47-63, wherein for each of the component virus groups, the vaccine comprises the HA protein(s), or the HA1-containing portion(s) thereof, of only one viral strain for each group.

65. A method of eliciting an immune response, comprising administering an immunogenic vaccine composition according to any one of claims 47-64 to a subject, thereby eliciting in the subject an immune response against influenza.

66. The method of claim 65, wherein eliciting the immune response comprises eliciting an Influenza B virus-specific immune response, and/or a pan-Influenza B virus-specific immune response.

67. The method of claim 66, wherein eliciting the immune response additionally comprises eliciting an immune response to at least one non-Influenza B vaccine strain.

68. The method of any one of claims 65-67, wherein the immune response comprises one or more of an antibody, a B cell, and/or a T cell response.

69. The method of any one of claims 65-68, wherein administration comprises administering the vaccine in one or more components administered together, or sequentially.


SEQUENCE LISTING (556 sequences)

SEQ ID NO: 1 - protein, 50 aa, synthetic construct
PNKKGSSPKLSKSTGTDQQSLYQNAVNNKGSSKEPGSYAGASRDLLTASS

SEQ ID NO: 2 - protein, 50 aa, synthetic construct
PNKKGDSPKLTNSSSDEQQSLYSNGVNNKGSSNEPGSHRGKSKDLLPARS

SEQ ID NO: 3 - protein, 50 aa, synthetic construct
PNEKEGSPKLKNSNSKDQQNIYQNEVNKKGTSNKPGSHAGKSRDLLPVRS

SEQ ID NO: 4 - protein, 50 aa, synthetic construct
PKEKDGSPNLNNSNIKDQQTLYQKEVNKKGSSNKPGSHAGKSRGLLSERS

SEQ ID NO: 5 - protein, 50 aa, synthetic construct
PKETNGSPKLSKSNIEDQKTLYRKEVNNKESSNEPGSHAGKSRGLLSKRS

SEQ ID NO: 6 - protein, 50 aa, synthetic construct
PNEANGSPNLSRSNIEEQRALYRKDVNNQESSNEPGPHARKSRDLLSNRS

SEQ ID NO: 7 - protein, 50 aa, synthetic construct
PNKKENSPKLSKSTSTDQQSLYQNAVNNKGSSKEPGPYAGANRGLLTVSS

SEQ ID NO: 8 - protein, 50 aa, synthetic construct
PKEKNGSPNLSKSNIEDQKTIYRKEVNNKESSNEPGSHKGKSRGLFSKKS

SEQ ID NO: 9 - protein, 50 aa, synthetic construct; identical to SEQ ID NO: 8

SEQ ID NO: 10 - protein, 50 aa, synthetic construct
PKEKNGSPNLSKSNIEDQKTIYRKEVNNKESSHEPGSHKGKSRNLFSKKS

SEQ ID NO: 11 - protein, 50 aa, synthetic construct
PNKKNGSPNLSKSNIGDQRAIYHTEVNNKESSHEPGSHKGRSRDLFSKKS

SEQ ID NO: 12 - protein, 50 aa, synthetic construct
PNEKNGSPTLSKSNIGDQRAIYHTEVNNKESSHEPGSHKGKSRGLFSKKS

SEQ ID NO: 13 - protein, 50 aa, synthetic construct
PNEKNGLPNLSNSNIRDQRAIYHTEVNNKESSHEPGSHNGKSRGLISKES

SEQ ID NO: 14 - protein, 50 aa, synthetic construct
PNGKNGLPNLSKSNIGNQRALYHTEVNNKESSHEPGSHNGKSRDLISKES

SEQ ID NO: 15 - protein, 50 aa, synthetic construct
PNGKNGLPNLSKSNIGDQRALYHKEANNKESSHEPGSHNGESRDLISRES

SEQ ID NO: 16 - protein, 50 aa, synthetic construct
PNGKNGLPNLSKSNIGDQKALYHTEANNKESSHEPGSHNGESRDLISKES

SEQ ID NO: 17 - protein, 50 aa, synthetic construct
PNKKGNSPKLSKSTSADQQSLYQNAINDKGSSREPGPHAGAKRDLSTASS

SEQ ID NO: 18 - protein, 50 aa, synthetic construct
PNKKGNSPKLSKSTSADQQSLYQNAINDKGTSREPGPHAGAKRDLSTASS

SEQ ID NO: 19 - protein, 50 aa, synthetic construct
PNKKGNSPKLNQSTTADQQSLYQNAINDKGTSREPGPHAGAKRDLSTASS

SEQ ID NO: 20 - protein, 50 aa, synthetic construct
PNKKGNSPKLSKSTESDQQTLYQNNTNNKGSSKDQGSHSGANRELLTANS

SEQ ID NO: 21 - protein, 50 aa, synthetic construct
PNKKGNSPKLNQTTTADQQSLYQNAINDKGTSREPGPHAGAKRDLSTARS

SEQ ID NO: 22 - protein, 50 aa, synthetic construct
PNKKGNSPKLNQTTIAAQESLYQNAINDKGTSREPGPHAGAKRDLSTARS

SEQ ID NO: 23 - protein, 50 aa, synthetic construct; identical to SEQ ID NO: 22

SEQ ID NO: 24 - protein, 50 aa, synthetic construct
  SITE 23: note = misc_feature - Xaa can be any naturally occurring amino acid
PNKKGDSPKLTNSSSDEQQSLYXNGVNNKGSSNEPGSHRGKSRDLLPARS

SEQ ID NO: 25 - protein, 50 aa, synthetic construct
  SITE 6: note = misc_feature - Xaa can be any naturally occurring amino acid
  SITE 49: note = misc_feature - Xaa can be any naturally occurring amino acid
PDEKEXSPKLKNSNSKEQQNLYQNEVNKKGTSNKPGSHEGKNRDLLPVXS

SEQ ID NO: 26 - protein, 50 aa, synthetic construct
PNKKEDSPKLKNSNSKEQQTLYQNEVNKKGSSNEPGSHAGKSRDLLPARS

SEQ ID NO: 27 - protein, 50 aa, synthetic construct
PNKKGGSPKLKNSNSKDQQTLYQNEVNKKGSSNEPGSHAGKSRDLLPARS

SEQ ID NO: 28 - protein, 50 aa, synthetic construct
PNEKDGSPNLNKSNIENQKTLYRKEVNKKESSNEPGSHAGKSRGLLSKRS

SEQ ID NO: 29 - protein, 50 aa, synthetic construct
PKEKDGSPNLNNSNIKDQQTLYQKEVNKKGSSNEPGSHAGKCRGLLSERS

SEQ ID NO: 30 - protein, 50 aa, synthetic construct; identical to SEQ ID NO: 4

SEQ ID NO: 31 - protein, 50 aa, synthetic construct; identical to SEQ ID NO: 4

SEQ ID NO: 32 - protein, 50 aa, synthetic construct
PKEKDGSPNLNKSNIEDQKTLYRKEVNKKESSNEPGSHAGKSRGLLSKRS

SEQ ID NO: 33 - protein, 50 aa, synthetic construct; identical to SEQ ID NO: 5

SEQ ID NO: 34 - protein, 50 aa, synthetic construct
PKEKNGSPNLNKSNIEDQKTLYRKEVNNKESSNEPGSHKGKSRGLFSKKS

SEQ ID NO: 35 - protein, 50 aa, synthetic construct
PKEKDGSPNLSKSNIEDQKTLYRKEVNNKESSNEPGSHKGKSRGLFSKKS

SEQ ID NO: 36 - protein, 50 aa, synthetic construct
  SITE 4: note = misc_feature - Xaa can be any naturally occurring amino acid
PKEXNGSPNLSKSNIEDQKTLYRKEVNNKESSNEPGSHAGKSRGLLSKRS

SEQ ID NO: 37 - protein, 50 aa, synthetic construct
PKEANGSPNLSKSNIEDQRTLYRKEVNNKESSNEPGSHAKKSRGLLSNRS

SEQ ID NO: 38 - protein, 50 aa, synthetic construct
PKEANGSPNLSKSNIEDQRTLYRKDVNNQESSNEPGSHARKSRDLLSNRS

SEQ ID NO: 39 - protein, 50 aa, synthetic construct; identical to SEQ ID NO: 37

SEQ ID NO: 40 - protein, 50 aa, synthetic construct; identical to SEQ ID NO: 6

SEQ ID NO: 41 - protein, 50 aa, synthetic construct
PNKKGNSPKLSKSTSTDQQSLYQNAVNNKGSSKEPGPYAGANRGLLTVSS

SEQ ID NO: 42 - protein, 50 aa, synthetic construct; identical to SEQ ID NO: 8

SEQ ID NO: 43 - protein, 50 aa, synthetic construct; identical to SEQ ID NO: 8

SEQ ID NO: 44 - protein, 50 aa, synthetic construct; identical to SEQ ID NO: 8

SEQ ID NO: 45 - protein, 50 aa, synthetic construct
PKEKNGSPNLSKSNIEDQKTIYRKEVNNKESSHEPGSHKGKSRDLFSKKS

SEQ ID NO: 46 - protein, 50 aa, synthetic construct
PKEKNGSPNLSKSNIEDQKTIYRKEVNNKESSHEPGSHKGKCRDLFSKKS

SEQ ID NO: 47 - protein, 50 aa, synthetic construct; identical to SEQ ID NO: 45

SEQ ID NO: 48 - protein, 50 aa, synthetic construct; identical to SEQ ID NO: 10

SEQ ID NO: 49 - protein, 50 aa, synthetic construct
PNEKEGSPNLSKSNIEDQKTIYRKEVNNKESSHEPGSHKGKSRDLFSKKS

SEQ ID NO: 50 - protein, 50 aa, synthetic construct
PKEKNGSPNLSSSNIEDQKTIYRKEVNNKESSNEPGSHKGKSRGLFSKKS

SEQ ID NO: 51 - protein, 50 aa, synthetic construct
PNKKNGSPNLSKSNIGDQRAIYHTEVNNKESSHEPGSHKGKSRDLFSKKS

SEQ ID NO: 52 - protein, 50 aa, synthetic construct
PNEKNGSPNLSKSNIGDQRAIYHTEVNNKESSHEPESHKGKSRDLFSKKS

SEQ ID NO: 53 - protein, 50 aa, synthetic construct
  SITE 5: note = misc_feature - Xaa can be any naturally occurring amino acid
  SITE 16: note = misc_feature - Xaa can be any naturally occurring amino acid
  SITE 48: note = misc_feature - Xaa can be any naturally occurring amino acid
PNEKXGSPKLSKSNSXDQQSLYQNEVNNKESSNEPGSHEGKSRGLFTXKS

SEQ ID NO: 54 - protein, 50 aa, synthetic construct
PNEKNGLPNLSKSNIGDQRAIYHTEVNNKESSHEPGSHNGKSRGLFSKES

SEQ ID NO: 55 - protein, 50 aa, synthetic construct
  SITE 16: note = misc_feature - Xaa can be any naturally occurring amino acid
PNKKNGLPNLSKSNIXDQRAIYHTEVNNKESSHEPGSHNGKSRGLFSKES

SEQ ID NO: 56 - protein, 50 aa, synthetic construct
PNKKNGLPNLSKSNIGDQRAIYHTEVNNKESSHEPGSHNGKSRDLFSKES

SEQ ID NO: 57 - protein, 50 aa, synthetic construct
PNEKNGLPNLSKSNMGDQRAIYHTEVNNKESSHEPGSHNGKSRGLFTKES

SEQ ID NO: 58 - protein, 50 aa, synthetic construct
PNKKGNSPKLSKSTSTDQQSLYQNAVNNKESSKEPGPYAGANRGLFTASS

SEQ ID NO: 59 - protein, 50 aa, synthetic construct
PNEKNGLPNLSKSNIGDQRAIYHTEVNNKESSHEPGSHNGKSRDLFSKES

SEQ ID NO: 60 - protein, 50 aa, synthetic construct; identical to SEQ ID NO: 59

SEQ ID NO: 61 - protein, 50 aa, synthetic construct
PNEKNGLPNLSKSNIGDQRAIYHTEVNNKESSHEPGSHNGKSRNLISKES

SEQ ID NO: 62 - protein, 50 aa, synthetic construct
PNEKNGLPNLSKSNIGDQRAIYHTEVNNKESSHEPGSHNGKSRDLISKES

SEQ ID NO: 63 - protein, 50 aa, synthetic construct
PNGKNGLPNLSKSNIGDQRALYHTEVNNKESSHEPGSHNGKSRDLISKES

SEQ ID NO: 64 - protein, 50 aa, synthetic construct
PNGKNGLPNLSKSNIGDQRALYHTEANNKESSHEPGSHNGKSRDLISKES

SEQ ID NO: 65 - protein, 50 aa, synthetic construct
PNGKNGLPNLSMSNIGDQRALYHTEVNNKESSHEPGSHNGKSRDLISKES

SEQ ID NO: 66 - protein, 50 aa, synthetic construct
PNGKNGLPNLSKSNIGBQRALYHTEVNNKESSHEPGSHNGKSRDLISKES

SEQ ID NO: 67 - protein, 50 aa, synthetic construct; identical to SEQ ID NO: 64

SEQ ID NO: 68 - protein, 50 aa, synthetic construct; identical to SEQ ID NO: 64

SEQ ID NO: 69 - protein, 50 aa, synthetic construct; identical to SEQ ID NO: 64

SEQ ID NO: 70 - protein, 50 aa, synthetic construct; identical to SEQ ID NO: 64

SEQ ID NO: 71 - protein, 50 aa, synthetic construct; identical to SEQ ID NO: 16

SEQ ID NO: 72 - protein, 50 aa, synthetic construct
PNGKNGLPNLSKSNIGDQKTLYHTEANNKESSHEPGSHNGESRDLISKES

SEQ ID NO: 73 - protein, 50 aa, synthetic construct; identical to SEQ ID NO: 18

SEQ ID NO: 74 - protein, 50 aa, synthetic construct; identical to SEQ ID NO: 18

SEQ ID NO: 75 - protein, 50 aa, synthetic construct
PNKKGNSPKLSKSTTADQQSLYQNAINDKGTSREPGPHAGAKRDLSTASS

SEQ ID NO: 76 - protein, 50 aa, synthetic construct; identical to SEQ ID NO: 75

SEQ ID NO: 77 - protein, 50 aa, synthetic construct; identical to SEQ ID NO: 75

SEQ ID NO: 78 - protein, 50 aa, synthetic construct
PNKKGNSPKLSQSTTADQQSLYQNAINDKGTSREPGPHAGAKRDLSTASS

SEQ ID NO: 79 - protein, 50 aa, synthetic construct; identical to SEQ ID NO: 78

SEQ ID NO: 80 - protein, 50 aa, synthetic construct; identical to SEQ ID NO: 19

SEQ ID NO: 81 - protein, 50 aa, synthetic construct; identical to SEQ ID NO: 21

SEQ ID NO: 82 - protein, 50 aa, synthetic construct; identical to SEQ ID NO: 21

SEQ ID NO: 83 - protein, 50 aa, synthetic construct; identical to SEQ ID NO: 21

SEQ ID NO: 84 - protein, 50 aa, synthetic construct
PNKKGNSPKINQTTIADQQSLYQNAINDKGTSREPGPHAGAKRDLSTARS

SEQ ID NO: 85 - protein, 50 aa, Influenza A Virus
PNKKGDSPKLKNSNSKEQQSLYQNEVNKKGSSNEPGSHAGKSRDLLPARS

SEQ ID NO: 86 - protein, 50 aa, Influenza A Virus
PKEKNGSPNLSKSNIEDQKTLYRKEVNNKESSNEPGSHKGKSRGLFSKKS

SEQ ID NO: 87 - protein, 50 aa, Influenza A Virus; identical to SEQ ID NO: 63

SEQ ID NO: 88 - protein, 50 aa, Influenza A Virus
PNKKGNSPKLNKSTTADQQSLYQNAINDKGTSREPGPHAGAKRDLSTASS

SEQ ID NO: 89 - protein, 252 aa, Influenza A Virus
LEDSHNGKLCKLKGIAPLQLGKCNIAGWLLGNPECDLLLTASSWSYIVETSNSENGTCYPGDFIDYEELREQLSSVSSFEKFEIFPKTSSWPNHETTKGVTAACSYAGASSFYRNLLWLTKKGSSYPKLSKSYVNNKGKEVLVLWGVHHPPTGTDQQSLYQNADAYVSVGSSKYNRRFTPEIAARPKVRDQAGRMNYYWTLLEPGDTITFEATGNLIAPWYAFALNRGSGSGIITSDAPVHDCNTKCQTPHG

SEQ ID NO: 90 - protein, 251 aa, Influenza A Virus
LEDRHNGKLCKLKGIAPLQLGKCNIIGWLLGNPECDSLLPARSWSYIVETPNSENGACYPGDFIDYEELREQLSSVSSLERFEIFPKESSWPNHTFNGVTASCSHRGKSSFYRNLLWLTKKGDSYPKLTNSYVNNKGKEVLVLWGVHHPSSSDEQQSLYSNGNAYVSVASSNYNRRFTPEIAARPKVKDQHGRMNYYWTLLEPGDTIIFEATGNLIAPWYAFALSRGFESGIITSNASMHECNTKCQTPQG

SEQ ID NO: 91 - protein, 252 aa, Influenza A Virus
LEDSHNGKLCRLKGIAPLQLGKCNIAGWLLGNPECDPLLPVRSWSYIVETPNSENGICYPGDFIDYEELREQLSSVSSFERFEIFPKESSWPNHNTTKGVTAACSHAGKSSFYRNLLWLTEKEGSYPKLKNSYVNKKGKEVLVLWGIHHPSNSKDQQNIYQNENAYVSVVTSNYNRRFTPEIAERPKVRDQAGRMNYYWTLLKPGDTIIFEANGNLIAPRYAFALSRGFGSGIITSNASMHECNTKCQTPLG

SEQ ID NO: 92 - protein, 252 aa, Influenza A Virus
LEDSHNGKLCRLKGIAPLQLGKCNIAGWILGNPECESLLSERSWSYIVETPNSENGTCYPGDFIDYEELREQLSSVSSFERFEIFSKESSWPKHNTTRGVTAACSHAGKSSFYRNLLWLTEKDGSYPNLNNSYVNKKGKEVLVLWGVHHPSNIKDQQTLYQKENAYVSVVSSNYNRRFTPEIAERPKVRGQAGRMNYYWTLLKPGDTIMFEANGNLIAPWYAFALSRGFGSGIITSNASMHECDTKCQTPQG

SEQ ID NO: 93 - protein, 252 aa, Influenza A Virus
LEDSHNGKLCRLKGIAPLQLGKCNIAGWILGNPECESLLSKRSWSYIAETPNSENGTCYPGDFADYEELREQLSSVSSFERFEIFPKERSWPKHNITRGVTAACSHAGKSSFYKNLLWLTETNGSYPKLSKSYVNNKEKEVLVLWGVHHPSNIEDQKTLYRKENAYVSVVSSNYNRRFTPEIAERPKVRGQAGRMNYYWTLLEPGDTIIFEANGNLIAPWYAFALSRGFGSGIITSNASMDECDTKCQTPQG

SEQ ID NO: 94 - protein, 251 aa, Influenza A Virus
LEDSHNGKLCRLKGKAPLQLGNCNIAGWVLGNPECESLLSNRSWSYIAETPNSENGTCYPGDFADYEELREQLSSVSSFERFEIFPKERSWPNHTTRGVTAACPHARKSSFYKNLVWLTEANGSYPNLSRSYVNNQEKEVLVLWGVHHPSNIEEQRALYRKDNAYVSVVSSNYNRRFTPEIAKRPKVRDQSGRMNYYWTLLEPGDTIIFEATGNLIAPWYAFALSRGPGSGIITSNAPLDECDTKCQTPQG

SEQ ID NO: 95 - protein, 252 aa, Influenza A Virus
LEDRHNGKLCKLGGIAPLHLGKCNIAGWLLGNPECELLLTVSSWSYIVETSNSDNGTCYPGDFINYEELREQLSSVSSFERFEIFPKTSSWPNHETNRGVTAACPYAGANSFYRNLIWLVKKENSYPKLSKSYVNNKGKEVLVLWGIHHPPTSTDQQSLYQNADAYVFVGSSKYNRKFKPEIAARPKVRGQAGRMNYYWTLIEPGDTITFEATGNLVVPRYAFAMNRGSGSGIIISDAPVHDCNTKCQTPKG

SEQ ID NO: 96 - protein, 252 aa, Influenza A Virus
LEDSHNGKLCRLKGIAPLQLGKCNIAGWILGNPECESLFSKKSWSYIAETPNSENGTCYPGYFADYEELREQLSSVSSFERFEIFPKERSWPKHNVTRGVTASCSHKGKSSFYRNLLWLTEKNGSYPNLSKSYVNNKEKEVLVLWGVHHPSNIEDQKTIYRKENAYVSVVSSNYNRRFTPEIAERPKVRGQAGRINYYWTLLEPGDTIIFEANGNLIAPWHAFALNRGFGSGIITSNASMDECDTKCQTPQG

SEQ ID NO: 97 - protein, 252 aa, Influenza A Virus
LEDSHNGKLCRLKGIAPLQLGKCSIAGWILGNPECESLFSKKSWSYIAETPNSENGTCYPGYFADYEELREQLSSVSSFERFEIFPKERSWPKHNITRGVTASCSHKGKSSFYRNLLWLTEKNGSYPNLSKSYVNNKEKEVLVLWGVHHPSNIEDQKTIYRKENAYVSVVSSNYNRRFTPEIAKRPKVRGQEGRINYYWTLLEPGDTIIFEANGNLIAPWYAFALSRGFGSGIITSNASMDECDTKCQTPQG

SEQ ID NO: 98 - protein, 252 aa, Influenza A Virus
LEDNHNGKLCKLKGIAPLQLGKCSIAGWILGNPECESLFSKKSWSYIAETPNSENGTCYPGYFADYEELREQLSSVSSFERFEIFPKESSWPKHNVTKGVTAACSHKGKSSFYRNLLWLTEKNGSYPNLSKSYVNNKEKEVLVLWGVHHPSNIEDQKTIYRKENAYVSVVSSHYNRRFTPEIAKRPKVRNQEGRINYYWTLLEPGDTIIFEANGNLIAPWYAFALSRGFGSGIITSNASMDECDAKCQTPQG

SEQ ID NO: 99 - protein, 252 aa, Influenza A Virus
LEDSHNGKLCRLKGIAPLQLGNCSIAGWILGNPECESLFSKKSWSYIAETPNSENGTCYPGYFADYEELREQLSSVSSFERFEIFPKESSWPNHTVTKGVTASCSHKGRSSFYRNLLWLTKKNGSYPNLSKSYVNNKEKEVLVLWGVHHPSNIGDQRAIYHTENAYVSVVSSHYNRRFTPEIAKRPKVRDQEGRINYYWTLLEPGDTIIFEANGNLIAPWYAFALSRGFGSGIITSNASMDECDAKCQTPQG

SEQ ID NO: 100 - protein, 252 aa, Influenza A Virus
LEDSHNGKLCRLKGIAPLQLGNCSIAGWILGNPECESLFSKKSWSYIAETPNSENGTCYPGYFADYEELREQLSSVSSFERFEIFPKESSWPNHTVTKGVTASCSHKGKSSFYRNLLWLTEKNGSYPTLSKSYVNNKEKEVLVLWGVHHPSNIGDQRAIYHTENAYVSVVSSHYNRRFTPEIAKRPKVRGQEGRINYYWTLLEPGDTIIFEANGNLIAPWYAFALSRGFGSGIITSNASMDECDAKCQTPQG

SEQ ID NO: 101 - protein, 251 aa, Influenza A Virus
LEDSHNGKLCLLKGIAPLQLGNCSVAGWILGNPECESLISKESWSYIVETPNPENGTCYPGYFADYEELREQLSSVSSFERFEIFPKESSWPNHTVTGVTASCSHNGKSSFYRNLLWLTEKNGLYPNLSNSYVNNKEKEVLVLWGVHHPSNIRDQRAIYHTENAYVSVVSSHYSRRFTPEIAKRPKVRGQEGRINYYWTLLEPGDTIIFEANGNLIAPWYAFALSRGFGSGIITSNAPMNECDAKCQTPQG

SEQ ID NO: 102 - protein, 251 aa, Influenza A Virus
LEDSHNGKLCLLKGIAPLQLGNCSVAGWILGNPECELLISKESWSYIVETPNPENGTCYPGYFADYEELREQLSSVSSFERFEIFPKESSWPNHTVTGVSASCSHNGKSSFYRNLLWLTGKNGLYPNLSKSYVNNKEKEVLVLWGVHHPPNIGNQRALYHTENAYVSVVSSHYSRRFTPEIAKRPKVRDQEGRINYYWTLLEPGDTIIFEANGNLIAPWYAFALSRGFGSGIITSNAPMDECDAKCQTPQG

SEQ ID NO: 103 - protein, 251 aa, Influenza A Virus
LEDSHNGKLCLLKGIAPLQLGNCSVAGWILGNPECELLISRESWSYIVEKPNPENGTCYPGHFADYEELREQLSSVSSFERFEIFPKESSWPNHTTTGVSASCSHNGESSFYKNLLWLTGKNGLYPNLSKSYANNKEKEVLVLWGVHHPPNIGDQRALYHKENAYVSVVSSHYSRKFTPEIAKRPKVRDQEGRINYYWTLLEPGDTIIFEANGNLIAPRYAFALSRGFGSGIINSNAPMDECDAKCQTPQG

SEQ ID NO: 104 - protein, 251 aa, Influenza A Virus
LENSHNGKLCLLKGIAPLQLGNCSVAGWILGNPECELLISKESWSYIVEKPNPENGTCYPGHFADYEELREQLSSVSSFERFEIFPKESSWPNHTVTGVSASCSHNGESSFYRNLLWLTGKNGLYPNLSKSYANNKEKEVLVLWGVHHPPNIGDQKALYHTENAYVSVVSSHYSRKFTPEIAKRPKVRDQEGRINYYWTLLEPGDTIIFEANGNLIAPRYAFALSRGFGSGIINSNAPMDKCDAKCQTPQG

SEQ ID NO: 105 - protein, 252 aa, Influenza A Virus
LEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRDQEGRMNYYWTLVEPGDKITFEATGNLVVPRYAFAMERNAGSGIIISDTPVHDCNTTCQTPKG

SEQ ID NO: 106 - protein, 252 aa, Influenza A Virus
  SITE 191: note = misc_feature - Xaa can be any naturally occurring amino acid
LEDKHNGKLCKLRGVAPLHLGKCNIAGWILGNPECESLSTASSWSYIVETSSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGTSRYSKKFKPEIAIRPKVRDXEGRMNYYWTLVEPGDKITFEATGNLVVPRYAFAMERNAGSGIIISDTPVHDCNTTCQTPKG

SEQ ID NO: 107 - protein, 252 aa, Influenza A Virus
LEDKHNGKLCKLRGVAPLHLGKCNIAGWILGNPECESLSTASSWSYIVETSNSDNGTCYPGDFINYEELREQLSSVSSFERFEIFPKTSSWPNHDSNKGVTAACPHAGAKSFYKNLIWLVKKGNSYPKLNQSYINDKGKEVLVLWGIHHPSTTADQQSLYQNADAYVFVGTSRYSKKFKPEIATRPKVRDQEGRMNYYWTLVEPGDKITFEATGNLVVPRYAFTMERNAGSGIIISDTPVHDCNTTCQTPEG

SEQ ID NO: 108 - protein, 252 aa, Influenza A Virus
LENSHNGKLCSLNGKIPLQLGNCNVAGWILGNPKCDLLLTANSWSYIIETSNSKNGACYPGEFADYEELKEQLSTVSSFERFEIFPKATSWPNHDTTRGTTVACSHSGANSFYRNLLWIVKKGNSYPKLSKSYTNNKGKEVLVIWGVHHPPTESDQQTLYQNNHTYVSVGSSKYYKRFTPEIVARPKVREQAGRMNYYWTLLDQGDTITFEATGNLIAPWHAFALKKGSSSGIMRSDAQVHNCTTKCQTPHG

SEQ ID NO: 109 - protein, 252 aa, Influenza A Virus
LEDKHNGKLCKLRGVAPLHLGKCNIAGWILGNPECESLSTARSWSYIVETSNSDNGTCYPGDFINYEELREQLSSVSSFERFEIFPKTSSWPNHDSNKGVTAACPHAGAKSFYKNLIWLVKKGNSYPKLNQTYINDKGKEVLVLWGIHHPPTTADQQSLYQNADAYVFVGTSRYSKKFKPEIATRPKVRDQEGRMNYYWTLVEPGDKITFEATGNLVVPRYAFTMERNAGSGIIISDTPVHDCNTTCQTPEG

SEQ ID NO: 110 - protein, 252 aa, Influenza A Virus
LEDKHNGKLCKLRGVAPLHLGKCNIAGWILGNPECESLSTARSWSYIVETSNSDNGTCYPGDFINYEELREQLSSVSSFERFEIFPKTSSWPNHDSDKGVTAACPHAGAKSFYKNLIWLVKKGNSYPKLNQTYINDKGKEVLVLWGIHHPPTIAAQESLYQNADAYVFVGTSRYSKKFKPEIATRPKVRDQEGRMNYYWTLVEPGDKITFEATGNLVVPRYAFTMERDAGSGIIISDTPVHDCNTTCQTPEG

SEQ ID NO: 111 - protein, 252 aa, Influenza A Virus; identical to SEQ ID NO: 110

SEQ ID NO: 112 - protein, 252 aa, Influenza A Virus
  SITE 26: note = misc_feature - Xaa can be any naturally occurring amino acid
  SITE 95: note = misc_feature - Xaa can be any naturally occurring amino acid
  SITE 161: note = misc_feature - Xaa can be any naturally occurring amino acid
LEDRHNGKLCKLKGIAPLQLGKCNIXGWLLGNPECDSLLPARSWSYIVETPNSENGACYPGDFIDYEELREQLSSVSSLERFEIFPKESSWPNHXTFNGVTASCSHRGKSSFYRNLLWLTKKGDSYPKLTNSYVNNKGKEVLVLWGVHHPSSSDEQQSLYXNGNAYVSVASSNYNRRFTPEIAARPKVRDQHGRMNYYWTLLEPGDTIIFEATGNLIAPWYAFALSRGFESGIITSNASMHECNTKCQTPQG

SEQ ID NO: 113 - protein, 252 aa, Influenza A Virus
  SITE 42: note = misc_feature - Xaa can be any naturally occurring amino acid
  SITE 97: note = misc_feature - Xaa can be any naturally occurring amino acid
  SITE 124: note = misc_feature - Xaa can be any naturally occurring amino acid
LEDSHNGKLCRLKGIAPLQLGKCNIAGWLLGNPECDPLLPVXSWSYIVETPNSENGICYPGDFIDYEELREQLSSVSSFERFEIFPKESSWPDHNTXNGVTAACSHEGKNSFYRNLLWLTEKEXSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSNASMHECNTKCQTPLG

SEQ ID NO: 114 - protein, 252 aa, Influenza A Virus
LEDSHNGKLCRLKGIAPLQLGKCNIAGWLLGNPECDSLLPARSWSYIVETPNSENGICYPGDFIDYEELREQLSSVSSFERFEIFPKESSWPNHNTTKGVTAACSHAGKSSFYRNLLWLTKKEDSYPKLKNSYVNKKGKEVLVLWGVHHPSNSKEQQTLYQNENAYVSVVSSNYNRRFTPEIAERPKVRDQTGRMNYYWTLLEPGDTIIFEANGNLIAPWYAFALSRGFGSGIITSNASMHECNTKCQTPQG

SEQ ID NO: 115 - protein, 252 aa, Influenza A Virus
LEDSHNGKLCRLKGIAPLQLGKCNIAGWLLGNPECDPLLPARSWSYIVETPNSENGICYPGAFIDYEELREQLSSVSSFERFEIFPKESSWPNHNTNIGVTAACSHAGKSSFYRNLLWLTKKGGSYPKLKNSYVNKKGKEVLVLWGIHHPSNSKDQQTLYQNENAYVSVVSSNYNRRFTPEIAERPEVRDQAGRMNYYWTLLEPGDTIMFEANGNLVAPWYAFALSRGFGSGIITSNASMHECNTKCQTPQG

SEQ ID NO: 116 - protein, 252 aa, Influenza A Virus
LEDSHNGKLCRLKGIAPLQLGKCNIAGWILGNPECESLLSKRSWSYIAETPNSENGTCYPGDFADYEELREQLSSVSSFERFEIFPKERSWPNHNINIGVTAACSHAGKSSFYKNLLWLTEKDGSYPNLNKSYVNKKEKEVLVLWGVHHPSNIENQKTLYRKENAYVSVVSSNYNRRFTPEIAERPKVRGQAGRMNYYWTLLEPGDTIIFEANGNLIAPWYAFALSRGLGSGIITSNASMDECDTKCQTPQG

SEQ ID NO: 117 - protein, 252 aa, Influenza A Virus
LEDSHNGKLCRLKGIAPLQLGKCNIAGWILGNPECESLLSERSWSYIVETPNSENGTCYPGDFIDYEELREQLSSVTSFERFEIFPKETSWPKHNTTKGVTAACSHAGKCSFYRNLLWLTEKDGSYPNLNNSYVNKKGKEVLVLWGVHHPSNIKDQQTLYQKENAYVSVVSSNYNRRFTPEIAERPKVRGQAGRMNYYWTLLEPGDTIIFEANGNLIAPWYAFALSRGFGSGIITSNASMHECNTKCQTPQG

SEQ ID NO: 118 - protein, 252 aa, Influenza A Virus; identical to SEQ ID NO: 92

SEQ ID NO: 119 - protein, 252 aa, Influenza A Virus
LEDSHNGKLCRLKGIAPLQLRKCNIAGWILGNPECESLLSERSWSYIVETPNSENGTCYPGDFTNYEELREQLSSVSSFERFEIFPKESSWPKHNTTRGVTAACSHAGKSSFYRNLLWLTEKDGSYPNLNNSYVNKKGKEVLVLWGVHHPSNIKDQQTLYQKENAYVSVVSSNYNRRFTPEIAERPKVRGQAGRMNYYWTLLKPGDTIMFEANGNLIAPWYAFALSRGFGSGIITSNASMHECDTKCQTPQG

SEQ ID NO: 120 - protein, 252 aa, Influenza A Virus
  SITE 57: note = misc_feature - Xaa can be any naturally occurring amino acid
LEDSHNGKLCRLKGIAPLQLGKCNIAGWILGNPECESLLSKRSWSYIAETPNSENGXCYPGDFADYEELREQLSSVSSFERFEIFPKERSWPKHNINIGVTAACSHAGKSSFYKNLLWLTEKDGSYPNLNKSYVNKKEKEVLVLWGVHHPSNIEDQKTLYRKENAYVSVVSSNYNRRFTPEIAERPKVRGQAGRINYYWTLLEPGDTIIFEANGNLIAPWYAFALSRGIGSGIITSNASMDECDTKCQTPQG

SEQ ID NO: 121 - protein, 252 aa, Influenza A Virus; identical to SEQ ID NO: 93

SEQ ID NO: 122 - protein, 252 aa, Influenza A Virus
LEDSHNGKLCRLKGIAPLQLGKCNIAGWILGNPECESLFSKKSWSYIAETPNSENGTCYPGYFADYEELREQLSSVSSFERFEIFPKERSWPKHNITRGVTAACSHKGKSSFYRNLLWLTEKNGSYPNLNKSYVNNKEKEVLVLWGVHHPSNIEDQKTLYRKENAYVSVVSSNYNRRFTPEIAERPKVRGQAGRINYYWTLLEPGDTIIFEANGNLIAPWHAFALSRGFGSGIITSNASMDECDTKCQTPQG

SEQ ID NO: 123 - protein, 252 aa, Influenza A Virus
LEDSHNGKLCRLKGIAPLQLGKCNIAGWILGNPECESLFSKKSWSYIAETPNSENGTCYPGYFADYEELREQLSSVSSFERFEIFPKERSWPKHNVTRGVTAACSHKGKSSFYRNLLWLTEKDGSYPNLSKSYVNNKEKEVLVLWGVHHPSNIEDQKTLYRKENAYVSVVSSNYNRRFTPEIAERPKVRGQAGRINYYWTLLEPGDTIIFEANGNLIAPWHAFALSRGFGSGIITSNASMDECDTKCQTPQG

SEQ ID NO: 124 - protein, 252 aa, Influenza A Virus
  SITE 122: note = misc_feature - Xaa can be any naturally occurring amino acid
  SITE 229: note = misc_feature - Xaa can be any naturally occurring amino acid
LEDSHNGKLCRLKGIAPLQLGKCNIAGWILGNPECESLLSKRSWSYIAETPNSENGTCYPGDFADYEELREQLSSVSSFERFEIFPKERSWPKHNITRGVTAACSHAGKSSFYKNLLWLTEXNGSYPNLSKSYVNNKEKEVLVLWGVHHPSNIEDQKTLYRKENAYVSVVSSNYNRRFTPEIAERPKVRGQAGRMNYYWTLLEPGDTIIFEANGNLIAPWYAFALSRGXGSGIITSNASMDECDTKCQTPQG

SEQ ID NO: 125 - protein, 252 aa, Influenza A Virus
LEDSHNGKLCRLKGIAPLQLGKCNIAGWILGNPECESLLSNRSWSYIAETPNSENGTCYPGDFADYEELREQLSSVSSFERFEIFPKERSWPKHNITRGVTAACSHAKKSSFYKNLLWLTEANGSYPNLSKSYVNNKEKEVLVLWGVHHPSNIEDQRTLYRKENAYVSVVSSNYNRRFTPEIAERPKVRGQAGRMNYYWTLLEPGDKIIFEANGNLIAPWYAFALSRGLGSGIITSNASMDECDTKCQTPQG

SEQ ID NO: 126 - protein, 252 aa, Influenza A Virus
LEDSHNGKLCRLKGKAPLQLGKCNIAGWVLGNPECESLLSNRSWSYIAETPNSENGTCYPGDFADYEELREQLSSVSSFERFEIFPKERSWPKHNTTRGVTAACSHARKSSFYKNLVWLTEANGSYPNLSKSYVNNQEKEVLVLWGVHHPSNIEDQRTLYRKDNAYVSVVSSNYNRRFTPEIAKRPKVRDQPGRMNYYWTLLEPGDTIIFEATGNLIAPWYAFALSRGPGSGIITSNAPMDECDTKCQTPQG

SEQ ID NO: 127 - protein, 252 aa, Influenza A Virus
LEDSHNGKLCRLKGIAPLQLGKCNIAGWILGNPECESLLSNRSWSYIAETPNSENGTCYPGDFADYEELREQLSSVSSFERFEIFPKERSWPKHNVTRGVTAACSHAKKSSFYKNLLWLTEANGSYPNLSKSYVNNKEKEVLVLWGVHHPSNIEDQRTLYRKENAYVSVVSSNYNRRFTPEIAERPKVRGQPGRMNYYWTLLEPGDTIIFEANGNLIAPWYAFALSRGPGSGIITSNASMDECDTKCQTPQG

SEQ ID NO: 128 - protein, 251 aa, Influenza A Virus; identical to SEQ ID NO: 94

SEQ ID NO: 129 - protein, 252 aa, Influenza A Virus
LEDRHNGKLCKLGGIAPLHLGKCNIAGWLLGNPECELLLTVSSWSYIVETSNSDNGTCYPGDFINYEELREQLSSVSSFERFEIFPKTSSWPNHETNRGVTAACPYAGANSFYRNLIWLVKKGNSYPKLSKSYVNNKGKEVLVLWGIHHPPTSTDQQSLYQNADAYVFVGSSKYNRKFKPEIAARPKVRGQAGRMNYYWTLIEPGDTITFEATGNLVVPRYAFAMNRGSGSGIIISDAPVHDCNTKCQTPKG

SEQ ID NO: 130 - protein, 252 aa, Influenza A Virus
LEDSHNGKLCRLKGIAPLQLGKCSIAGWILGNPECESLFSKKSWSYIAETPNSENGTCYPGYFADYEELREQLSSVSSFERFEIFPKERSWPKHNVTRGVTASCSHKGKSSFYRNLLWLTEKNGSYPNLSKSYVNNKEKEVLVLWGVHHPSNIEDQKTIYRKENAYVSVVSSNYNRRFTPEIAERPKVRGQAGRINYYWTLLEPGDTIIFEANGNLIAPWHAFALNRGFGSGIITSNASMDECDTKCQTPQG

SEQ ID NO: 131 - protein, 252 aa, Influenza A Virus
LEDSHNGKLCRLKGIAPLQLGKCSIAGWILGNPECESLFSKKSWSYIAETPNSENGTCYPGYFADYEELREQLSSVSSFERFEIFPKERSWPKHNVTRGVTASCSHKGKSSFYRNLLWLTEKNGSYPNLSKSYVNNKEKEVLVLWGVHHPSNIEDQKTIYRKENAYVSVVSSNYNRRFTPEIAERPKVRGQEGRINYYWTLLEPGDTIIFEANGNLIAPWYAFALSRGFGSGIITSNASMDECDTKCQTPQG

SEQ ID NO: 132 - protein, 252 aa, Influenza A Virus
LEDSHNGKLCRLKGIAPLQLGKCSIAGWILGNPECESLFSKKSWSYIAETPNSENGTCYPGYFADYEELREQLSSVSSFERFEIFPKERSWPKHNVTRGVTASCSHKGKSSFYRNLLWLTEKNGSYPNLSKSYVNNKEKEVLVLWGVHHPSNIEDQKTIYRKENAYVSVVSSNYNRRFTPEIAKRPKVRGQEGRINYYWTLLEPGDTIIFEANGNLIAPWYAFALSRGFGSGIITSNASMDECDTKCQTPQG

SEQ ID NO: 133 - protein, 252 aa, Influenza A Virus
LEDSHNGKLCRLKGIAPLQLGKCSIAGWILGNPECESLFSKKSWSYIAETPNSENGTCYPGYFADYEELREQLSSVSSFERFEIFPKESSWPKHNVTRGVTASCSHKGKSSFYRNLLWLTEKNGSYPNLSKSYVNNKEKEVLVLWGVHHPSNIEDQKTIYRKENAYVSVVSSHYNRRFTPEIAKRPKVRDQEGRINYYWTLLEPGDTIIFEANGNLIAPWYAFALSRGFGSGIITSNASMDECDAKCQTPQG

SEQ ID NO: 134 - protein, 252 aa, Influenza A Virus
LEDSHNGKLCRLKGIAPLQLGKCSIAGWILGNPECESLFSKKSWSYIAETPNSENGTCYPGYFADYEELREQLSSVSSFERFEIFPKESSWPKHNVTRGVTASCSHKGKCSFYRNLLWLTEKNGSYPNLSKSYVNNKEKEVLVLWGVHHPSNIEDQKTIYRKENAYVSVVSSHYNRRFTPEIAKRPKVRDQEGRINYYWTLLEPGDTIIFEANGNLIAPWYAFALSRGFGSGIITSNASMDECDAKCQTPQG

SEQ ID NO: 135 - protein, 252 aa, Influenza A Virus
LEDSHNGKLCRLKGIAPLQLGKCSIAGWILGNPECESLFSKKSWSYIAETPNSENGTCYPGYFADYEELREQLSSVSSFERFEIFPKESSWPKHSVTRGVTASCSHKGKSSFYRNLLWLTEKNGSYPNLSKSYVNNKEKEVLVLWGVHHPSNIEDQKTIYRKENAYVSVVSSHYNRRFTPEIAKRPKVRDQEGRINYYWTLLEPGDTIIFEANGNLIAPWYAFALSRGFGSGIITSNASMDECDAKCQTPQG

SEQ ID NO: 136 - protein, 252 aa, Influenza A Virus
LEDNHNGKLCKLKGIAPLQLGKCSIAGWILGNPECESLFSKKSWSYIAETPNSENGTCYPGYFADYEELREQLSSVSSFERFEIFPKESSWPKHNVTKGVTASCSHKGKSSFYRNLLWLTEKNGSYPNLSKSYVNNKEKEVLVLWGVHHPSNIEDQKTIYRKENAYVSVVSSHYNRRFTPEIAKRPKVRNQEGRINYYWTLLEPGDTIIFEANGNLIAPWYAFALSRGFGSGIITSNASMDECDAKCQTPQG

SEQ ID NO: 137 - protein, 252 aa, Influenza A Virus
  SITE 251: note = misc_feature - Xaa can be any naturally occurring amino acid
LEDNHNGKLCKLKGIAPLQLGKCSIAGWILGNPECESLFSKKSWSYIAETPNSENGTCYPGYFADYEELREQLSSVSSFERFEIFPKESSWPNHNVTKGVTASCSHKGKSSFYRNLLWLTEKEGSYPNLSKSYVNNKEKEVLVLWGIHHPSNIEDQKTIYRKENAYVSVVSSHYNRRFTPEIAKRPKVRDQEGRINYYWTLLEPGDTIIFEANGNLIAPWYAFALSRGFGSGIITSNASMDECDAKCQTPXG

SEQ ID NO: 138 - protein, 252 aa, Influenza A Virus
LEDSHNGKLCRLKGIAPLQLGKCSIAGWILGNPECESLFSKKSWSYIAETPNSENGTCYPGYFADYEELREQLSSVSSFERFEIFPKERSWPKHNVTRGVTASCSHKGKSSFYRNLLWLTEKNGSYPNLSSSYVNNKEKEVLVLWGVHHPSNIEDQKTIYRKENAYVSVVSSNYNRRFTPEIAERPKVRGQAGRINYYWTLLEPGDTIIFEANGNLIAPWHAFALNRGFGSGIITSNASMDECDTKCQTPQG

SEQ ID NO: 139 - protein, 252 aa, Influenza A Virus
LEDSHNGKLCRLKGIAPLQLGNCSIAGWILGNPECESLFSKKSWSYIAETPNSENGTCYPGYFADYEELREQLSSVSSFERFEIFPKESSWPNHTVTKGVTASCSHKGKSSFYRNLLWLTKKNGSYPNLSKSYVNNKEKEVLVLWGVHHPSNIGDQRAIYHTENAYVSVVSSHYNRRFTPEIAKRPKVRDQEGRINYYWTLLEPGDTIIFEANGNLIAPWYAFALSRGFGSGIITSNASMDECDAKCQTPQG

SEQ ID NO: 140 - protein, 252 aa, Influenza A Virus
LEDSHNGKLCRLKGIAPLQLGNCSIAGWILGNPECESLFSKKSWSYIAETPNSENGTCYPGYFADYEELREQLSSVSSFERFEIFPKESSWPNHTVTKGVTASCSHKGKSSFYRNLLWLTEKNGSYPNLSKSYVNNKEKEVLVLWGVHHPSNIGDQRAIYHTENAYVSVVSSHYNRRFTPEIAKRPKVRDQEGRINYYWTLLEPEDTIIFEANGNLIAPWYAFALSRGFGSGIITSNASMDECDAKCQTPQG

SEQ ID NO: 141 - protein, 252 aa, Influenza A Virus
  SITE 37: note = misc_feature - Xaa can be any naturally occurring amino acid
  SITE 41: note = misc_feature - Xaa can be any naturally occurring amino acid
  REGION 95..96: note = misc_feature - Xaa can be any naturally occurring amino acid
  SITE 123: note = misc_feature - Xaa can be any naturally occurring amino acid
  SITE 154: note = misc_feature - Xaa can be any naturally occurring amino acid
  SITE 220: note = misc_feature - Xaa can be any naturally occurring amino acid
LEDSHNGKLCRLKGIAPLQLGKCNIAGWLLGNPECEXLFTXKSWSYIVETPNSENGTCYPGDFIDYEELREQLSSVSSFERFEIFPKESSWPNHXXTRGVTAACSHEGKSSFYRNLLWLTEKXGSYPKLSKSYVNNKEKEVLVLWGIHHPPNSXDQQSLYQNENAYVSVVSSNYNRRFTPEIAERPKVRGQAGRMNYYWTLLEPGDTIIFEANGNLIAPXYAFALSRGFGSGIITSNASMHECNTKCQTPKG

SEQ ID NO: 142 - protein, 252 aa, Influenza A Virus
LEDSHNGKLCRLKGIAPLQLGNCSIAGWILGNPECESLFSKESWSYIAETPNSENGTCYPGYFADYEELREQLSSVSSFERFEIFPKESSWPNHTVTKGVTAACSHNGKSSFYRNLLWLTEKNGLYPNLSKSYVNNKEKEVLVLWGVHHPSNIGDQRAIYHTENAYVSVVSSHYSRRFTPEIAKRPKVRGQEGRINYYWTLLEPGDTIIFEANGNLIAPWYAFALSRGFGSGIITSNASMDECDAKCQTPQG

SEQ ID NO: 143 - protein, 252 aa, Influenza A Virus
  SITE 154: note = misc_feature - Xaa can be any naturally occurring amino acid
LEDSHNGKLCRLKGIAPLQLGNCSVAGWILGNPKCESLFSKESWSYIAETPNPENGTCYPGYFADYEELREQLSSVSSFERFEIFPKESSWPNHTVTKGVTTSCSHNGKSSFYRNLLWLTKKNGLYPNLSKSYVNNKEKEVLVLWGVHHPSNIXDQRAIYHTENAYVSVVSSHYSRRFTPEIAKRPKVRGQEGRINYYWTLLEPGDTIIFEANGNLIAPWYAFALSRGFGSGIITSNASMDECDAKCQTPQG

SEQ ID NO: 144 - protein, 252 aa, Influenza A Virus
LEDSHNGKLCRLKGIAPLQLGNCSVAGWILGNPECESLFSKESWSYIAETPDPENGTCYPGYFADYEELREQLSSVSSFERFEIFPKESSWPNHTVTKGVTASCSHNGKSSFYRNLLWLTKKNGLYPNLSKSYVNNKEKEVLVLWGVHHPSNIGDQRAIYHTENAYVSVVSSHYSRRFTPEIAKRPKVRDQEGRINYYWTLLEPGDTIIFEANGNLIAPWYAFALSRGFGSGIITSNASMDECDAKCQTPQG

SEQ ID NO: 145 - protein, 252 aa, Influenza A Virus
LEDSHNGKLCRLKGIAPLQLGNCSVAGWILGNPECESLFTKESWSYIAETPNPENGTCYPGYFADYEELREQLSSVSSFERFEIFPKESSWPNHTVTKGVTASCSHNGKSSFYRNLLWLTEKNGLYPNLSKSYVNNKEKEVLVLWGVHHPSNMGDQRAIYHTENAYVSVVSSHYSRRFTPEIAKRPKVRGQEGRINYYWTLLEPGDTIIFEANGNLIAPRYAFALSRGFGSGIITSNASMDECDAKCQTPQG

SEQ ID NO: 146 - protein, 252 aa, Influenza A Virus
LEDRHNGKLCKLRGVAPLHLGKCNIAGWLLGNPECELLFTASSWSYIVETSNSDNGTCYPGDFINYEELREQLSSVSSFERFEIFPKASSWPNHETNRGVTAACPYAGANSFYRNLIWLVKKGNSYPKLSKSYVNNKEKEVLVLWGIHHPPTSTDQQSLYQNADAYVFVGSSKYNKKFKPEIATRPKVRGQAGRMNYYWTLVEPGDTITFEATGNLVVPRYAFAMKRGSGSGIIISDTPVHDCNTTCQTPKG

SEQ ID NO: 147 - protein, 252 aa, Influenza A Virus
LEDSHNGKLCRLKGTAPLQLGNCSVAGWILGNPECESLFSKESWSYIAETPNPENGTCYPGYFADYEELREQLSSVSSFERFEIFPKESSWPNHTVTKGVTASCSHNGKSSFYKNLLWLTEKNGLYPNLSKSYVNNKEKEVLVLWGVHHPSNIGDQRAIYHTENAYVSVVSSHYSRRFTPEIAKRPKVRDQEGRINYYWTLLEPGDTIIFEANGNLIAPWYAFALSRGFGSGIITSNASMGECDAKCQTPQG

SEQ ID NO: 148 - protein, 252 aa, Influenza A Virus; identical to SEQ ID NO: 147

SEQ ID NO: 149 - protein, 252 aa, Influenza A Virus
  SITE 98: note = misc_feature - Xaa can be any naturally occurring amino acid
LEDSHNGKLCLLKGIAPLQLGNCSVAGWILGNPECESLISKESWSYIVETPNPENGTCYPGYFADYEELREQLSSVSSFERFEIFPKESSWPNHTVTXGVSASCSHNGKSSFYRNLLWLTEKNGLYPNLSKSYVNNKEKEVLVLWGVHHPSNIGDQRAIYHTENAYVSVVSSHYSRRFTPEIAKRPKVRNQEGRINYYWTLLEPGDTIIFEANGNLIAPWYAFALSRGFGSGIITSNAPMDECDAKCQTPQG

SEQ ID NO: 150 - protein, 252 aa, Influenza A Virus
  SITE 98: note = misc_feature - Xaa can be any naturally occurring amino acid
LEDSHNGKLCLLKGIAPLQLGNCSVAGWILGNPECELLISKESWSYIVETPNPENGTCYPGYFADYEELREQLSSVSSFERFEIFPKESSWPNHTVTXGVSASCSHNGKSSFYRNLLWLTEKNGLYPNLSKSYVNNKEKEVLVLWGVHHPSNIGDQRAIYHTENAYVSVVSSHYSRRFTPEIAKRPKVRDQEGRINYYWTLLEPGDTIIFEANGNLIAPWYAFALSRGFGSGIITSNAPMDECDAKCQTPQG

SEQ ID NO: 151 - protein, 251 aa, Influenza A Virus
LEDSHNGKLCLLKGIAPLQLGNCSVAGWILGNPECELLISKESWSYIVETPNPENGTCYPGYFADYEELREQLSSVSSFERFEIFPKESSWPNHTVTGVSASCSHNGKSSFYRNLLWLTGKNGLYPNLSKSYVNNKEKEVLVLWGVHHPPNIGDQRALYHTENAYVSVVSSHYSRRFTPEIAKRPKVRDQEGRINYYWTLLEPGDTIIFEANGNLIAPWYAFALSRGFGSGIITSNAPMDECDAKCQTPQG

SEQ ID NO: 152 - protein, 252 aa, Influenza A Virus
  SITE 98: note = misc_feature - Xaa can be any naturally occurring amino acid
LEDSHNGKLCLLKGIAPLQLGNCSVAGWILGNPECELLISKESWSYIVETPNPENGTCYPGYFADYEELREQLSSVSSFERFEIFPKESSWPNHTVTXGVSASCSHNGKSSFYRNLLWLTGKNGLYPNLSKSYANNKEKEVLVLWGVHHPPNIGDQRALYHTENAYVSVVSSHYSRRFTPEIAKRPKVRDQEGRINYYWTLLEPGDTIIFEANGNLIAPWYAFALSRGFGSGIITSNAPMDECDAKCQTPQG

SEQ ID NO: 153 - protein, 252 aa, Influenza A Virus
  SITE 98: note = misc_feature - Xaa can be any naturally occurring amino acid
LEDSHNGKLCLLKGIAPLQLGNCSVAGWILGNPECELLISKESWSYIVETPNPENGTCYPGYFADYEELREQLSSVSSFERFEIFPKGSSWPNHTVTXGVSASCSHNGKSSFYRNLLWLTGKNGLYPNLSMSYVNNKEKEVLVLWGVHHPPNIGDQRALYHTENAYVSVVSSHYSRRFTPEIAKRPKVRDQEGRINYYWTLLEPGDTIIFEANGNLIAPWYAFALSRGFGSGIITSNAPMDECDAKCQTPQG

SEQ ID NO: 154 - protein, 251 aa, Influenza A Virus
  SITE 219: note = misc_feature - Xaa can be any naturally occurring amino acid
LEDSHNGKLCLLKGIAPLQLGNCSVAGWILGNPECELLISKESWSYIVETPNPENGTCYPGYFADYEELREQLSSVSSFERFEIFPKESSWPNHTVTGVSASCSHNGKSSFYRNLLWLTGKNGLYPNLSKSYVNNKEKEVLVLWGVHHPPNIGBQRALYHTENAYVSVVSSHYSRRFTPEIAKRPKVRDQEGRINYYWTLLEPGDTIIFEANGNLIAPXYAFALSRGFGSGIITSNAPMDECDAKCQTPQG

SEQ ID NO: 155 - protein, 251 aa, Influenza A Virus
LEDSHNGKLCLLKGIAPLQLGNCSVAGWILGNPECELLISKESWSYIVETPNPENGTCYPGYFADYEELREQLSSVSSFERFEIFPKESSWPNHTVTGVSASCSHNGKSSFYRNLLWLTGKNGLYPNLSKSYANNKEKEVLVLWGVHHPPNIGDQRALYHTENAYVSVVSSHYSRRFTPEIAKRPKVRDQEGRINYYWTLLEPGDTIIFEANGNLIAPRYAFALSRGFGSGIITSNAPMDECDAKCQTPQG

SEQ ID NO: 156 - protein, 251 aa, Influenza A Virus
LEDSHNGKLCLLKGIAPLQLGNCSVAGWILGNPECELLISKESWSYIVEKPNPENGTCYPGHFADYEELREQLSSVSSFERFEIFPKESSWPNHTVTGVSASCSHNGKSSFYKNLLWLTGKNGLYPNLSKSYANNKEKEVLVLWGVHHPPNIGDQRALYHTENAYVSVVSSHYSRKFTPEIAKRPKVRDQEGRINYYWTLLEPGDTIIFEANGNLIAPRYAFALSRGFGSGIINSNAPMDECDAKCQTPQG

SEQ ID NO: 157 - protein, 252 aa, Influenza A Virus
  SITE 96: note = misc_feature - Xaa can be any naturally occurring amino acid
LEDSHNGKLCLLKGIAPLQLGNCSVAGWILGNPECELLISKESWSYIVETPNPENGTCYPGYFADYEELREQLSSVSSFERFEIFPKESSWPNHTXVTGVSASCSHNGKSSFYRNLLWLTGKNGLYPNLSKSYANNKEKEVLVLWGVHHPPNIGDQRALYHTENAYVSVVSSHYSRRFTPEIAKRPKVRDQEGRINYYWTLLEPGDTIIFEANGNLIAPRFAFALSRGFGSGIITSNAPMDECDAKCQTPQG

SEQ ID NO: 158 - protein, 251 aa, Influenza A Virus; identical to SEQ ID NO: 156

SEQ ID NO: 159 - protein, 252 aa, Influenza A Virus
  SITE 97: note = misc_feature - Xaa can be any naturally occurring amino acid
LEDSHNGKLCLLKGIAPLQLGNCSVAGWILGNPECELLISKESWSYIVEKPNPENGTCYPGHFADYEELREQLSSVSSFERFEIFPKESSWPNHTVXTGVSASCSHNGESSFYRNLLWLTGKNGLYPNLSKSYANNKEKEVLVLWGVHHPPNIGDQKALYHTENAYVSVVSSHYSRKFTPEIAKRPKVRDQEGRINYYWTLLEPGDTIIFEANGNLIAPRYAFALSRGFGSGIINSNAPMDECDAKCQTPQG

SEQ ID NO: 160 - protein, 252 aa, Influenza A Virus
  SITE 97: note = misc_feature - Xaa can be any naturally occurring amino acid
LENSHNGKLCLLKGIAPLQLGNCSVAGWILGNPECELLISKESWSYIVEKPNPENGTCYPGHFADYEELREQLSSVSSFERFEIFPKESSWPNHTVXTGVSASCSHNGESSFYRNLLWLTGKNGLYPNLSKSYANNKEKEVLVLWGVHHPPNIGDQKTLYHTENAYVSVVSSHYSRKFTPEIAKRPKVRDQEGRINYYWTLLEPGDTIIFEANGNLIAPRYAFALSRGFGSGIINSNAPMDKCDAKCQTPQG

SEQ ID NO: 161 - protein, 252 aa, Influenza A Virus
LEDKHNGKLCKLRGVAPLHLGKCNIAGWILGNPECESLSTASSWSYIVETSSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGTSRYSKKFKPEIAIRPKVRDQEGRMNYYWTLVEPGDKITFEATGNLVVPRYAFAMERNAGSGIIISDTPVHDCNTTCQTPKG

SEQ ID NO: 162 - protein, 252 aa, Influenza A Virus; identical to SEQ ID NO: 161

SEQ ID NO: 163 - protein, 252 aa, Influenza A Virus
LEDKHNGKLCKLRGVAPLHLGKCNIAGWILGNPECESLSTASSWSYIVETSSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTTADQQSLYQNADAYVFVGTSRYSKKFKPEIAIRPKVRDQEGRMNYYWTLVEPGDKITFEATGNLVVPRYAFAMERNAGSGIIISDTPVHDCNTTCQTPKG

SEQ ID NO: 164 - protein, 252 aa, Influenza A Virus
LEDKHNGKLCKLRGVAPLHLGKCNIAGWILGNPECESLSTASSWSYIVETSSSDNGTCYPGDFIDYEELREQLSSVSSFERFEIFPKTSSWPNHDSNKGVTAACPHAGAKGFYKNLIWLVKKGNSYPKLSKSYINDKGKEVLVLWGIHHPSTTADQQSLYQNADTYVFVGTSRYSKKFKPEIAIRPKVRDQEGRMNYYWTLVEPGDKITFEATGNLVVPRYAFAMERNAGSGIIISDTPVHDCNTTCQTPKG

SEQ ID NO: 165 - protein, 252 aa, Influenza A Virus
LEDKHNGKLCKLRGVAPLHLGKCNIAGWILGNPECESLSTASSWSYIVETSSSDNGTCYPGDFINYEELREQLSSVSSFERFEIFPKTSSWPNHDSNKGVTAACPHAGAKSFYKNLIWLVKKGNSYPKLSKSYINDKGKEVLVLWGIHHPSTTADQQSLYQNADAYVFVGTSRYSKKFKPEIAIRPKVRDQEGRMNYYWTLVEPGDKITFEATGNLVVPRYAFAMERNAGSGIIISDTPVHDCNTTCQTPEG

SEQ ID NO: 166 - protein, 252 aa, Influenza A Virus
LEDKHNGKLCKLRGVAPLHLGKCNIAGWILGNPECESLSTASSWSYIVETSSSDNGTCYPGDFINYEELREQLSSVSSFERFEIFPKTSSWPNHDSNKGVTAACPHAGAKSFYKNLIWLVKKGNSYPKLSQSYINDKGKEVLVLWGIHHPSTTADQQSLYQNADAYVFVGTSRYSKKFKPEIAIRPKVRDQEGRMNYYWTLVEPGDKITFEATGNLVVPRYAFTMERNAGSGIIISDTPVHDCNTTCQTPEG

SEQ ID NO: 167 - protein, 252 aa, Influenza A Virus
LEDKHNGKLCKLRGVAPLHLGKCNIAGWILGNPECESLSTASSWSYIVETSNSDNGTCYPGDFINYEELREQLSSVSSFERFEIFPKTSSWPNHDSNKGVTAACPHAGAKSFYKNLIWLVKKGNSYPKLSQSYINDKGKEVLVLWGIHHPSTTADQQSLYQNADAYVFVGTSRYSKKFKPEIAIRPKVRDQEGRMNYYWTLVEPGDKITFEATGNLVVPRYAFTMERNAGSGIIISDTPVHDCNTTCQTPEG

SEQ ID NO: 168 - protein, 252 aa, Influenza A Virus; identical to SEQ ID NO: 107

SEQ ID NO: 169 - protein, 252 aa, Influenza A Virus
LEDKHNGKLCKLRGVAPLHLGKCNIAGWILGNPECESLSTARSWSYIVETSNSDNGTCYPGDFINYEELREQLSSVSSFERFEIFPKTSSWPNHDSNKGVTAACPHAGAKSFYKNLIWLVKKGNSYPKLNQTYINDKGKEVLVLWGIHHPSTTADQQSLYQNADAYVFVGTSRYSKKFKPEIATRPKVRDQEGRMNYYWTLVEPGDKITFEATGNLVVPRYAFTMERNAGSGIIISDTPVHDCNTTCQTPEG

SEQ ID NO: 170 - protein, 252 aa, Influenza A Virus; identical to SEQ ID NO: 109

SEQ ID NO: 171 - protein, 252 aa, Influenza A Virus
LEDKHNGKLCKLRGVAPLHLGKCNIAGWILGNPECESLSTARSWSYIVETSNSDNGTCYPGDFINYEELREQLSSVSSFERFEIFPKTSSWPNHDSNKGVTAACPHAGAKSFYKNLIWLVKKGNSYPKLNQTYINDKGKEVLVLWGIHHPPTTADQQSLYQNADAYVFVGTSRYSKKFKPEIATRPKVRDQEGRMNYYWTLVEPGDKITFEATGNLVVPRYAFTMERDAGSGIIISDTPVHDCNTTCQTPEG

SEQ ID NO: 172 - protein, 252 aa, Influenza A Virus
LEDKHNGKLCKLRGVAPLHLGKCNIAGWILGNPECESLSTARSWSYIVETSNSDNGTCYPGDFINYEELREQLSSVSSFERFEIFPKTSSWPNHDSDNGVTAACPHAGAKSFYKNLIWLVKKGNSYPKINQTYINDKGKEVLVLWGIHHPPTIADQQSLYQNADAYVFVGTSRYSKKFKPEIATRPKVRDQEGRMNYYWTLVEPGDKITFEATGNLVVPRYAFTMERDAGSGIIISDTPVHDCNTTCQTPEG

SEQ ID NO: 173 - protein, 252 aa, Influenza A Virus
  SITE 97: note = misc_feature - Xaa can be any naturally occurring amino acid
LEDSHNGKLCRLKGIAPLQLGKCNIAGWLLGNPECDPLLPARSWSYIVETPNSENGICYPGDFIDYEELREQLSSVSSFERFEIFPKESSWPNHNTXNGVTAACSHAGKSSFYRNLLWLTKKGDSYPKLKNSYVNKKGKEVLVLWGVHHPSNSKEQQSLYQNENAYVSVVSSNYNRRFTPEIAERPKVRDQAGRMNYYWTLLEPGDTIIFEANGNLIAPWYAFALSRGFGSGIITSNASMHECNTKCQTPQG

SEQ ID NO: 174 - protein, 252 aa, Influenza A Virus
LEDSHNGKLCRLKGIAPLQLGKCNIAGWILGNPECESLLSKRSWSYIAETPNSENGTCYPGDFADYEELREQLSSVSSFERFEIFPKERSWPKHNVTRGVTAACSHAGKSSFYRNLLWLTEKNGSYPNLSKSYVNNKEKEVLVLWGVHHPSNIEDQKTLYRKENAYVSVVSSNYNRRFTPEIAERPKVRGQAGRMNYYWTLLEPGDTIIFEANGNLIAPWYAFALSRGFGSGIITSNASMDECDTKCQTPQG

SEQ ID NO: 175 - protein, 252 aa, Influenza A Virus
  SITE 151: note = misc_feature - Xaa can be any naturally occurring amino acid
LEDSHNGKLCRLKGIAPLQLGNCSVAGWILGNPECESLFSKESWSYIVETPNPENGTCYPGYFADYEELREQLSSVSSFERFEIFPKESSWPNHTVTKGVTASCSHNGKSSFYRNLLWLTEKNGLYPNLSKSYVNNKEKEVLVLWGVHHPXNIGDQRAIYHTENAYVSVVSSHYSRRFTPEIAKRPKVRDQEGRINYYWTLLEPGDTIIFEANGNLIAPWYAFALSRGFGSGIITSNAPMDECDAKCQTPQG

SEQ ID NO: 176 - protein, 252 aa, Influenza A Virus
  SITE 151: note = misc_feature - Xaa can be any naturally occurring amino acid
  SITE 184: note = misc_feature - Xaa can be any naturally occurring amino acid
  SITE 224: note = misc_feature - Xaa can be any naturally occurring amino acid
LEDKHNGKLCKLRGVAPLHLGKCNIAGWILGNPECESLSTASSWSYIVETSNSDNGTCYPGDFINYEELREQLSSVSSFERFEIFPKTSSWPNHDSNKGVTAACPHAGAKSFYKNLIWLVKKGNSYPKLNKSYINDKGKEVLVLWGIHHPXTTADQQSLYQNADAYVFVGTSRYSKKFKPEIAXRPKVRDQEGRMNYYWTLVEPGDKITFEATGNLVVPRYAFXMERNAGSGIIISDTPVHDCNTTCQTPEG

SEQ ID NO: 177 - protein, 565 aa, Influenza A Virus
MKAKLLVLLYAFVATDADTICIGYHANNSTDTVDTIFEKNVAVTHSVNLLEDRHNGKLCKLKGIAPLQLGKCNIIGWLLGNPECDSLLPARSWSYIVETPNSENGACYPGDFIDYEELREQLSSVSSLERFEIFPKESSWPNHTFNGVTASCSHRGKSSFYRNLLWLTKKGDSYPKLTNSYVNNKGKEVLVLWGVHHPSSSDEQQSLYSNGNAYVSVASSNYNRRFTPEIAARPKVKDQHGRMNYYWTLLEPGDTIIFEATGNLIAPWYAFALSRGFESGIITSNASMHECNTKCQTPQGSINSNLPFQNIHPVTIGECPKYVRSTKLRMVTGLRNIPSIQYRGLFGAIAGFIEGGWTGMIDGWYGYHHQNEQGSGYAADQKSTQNAINGITNKVNSIIEKMNTQFTAVGKEFNNLEKRMENLNKKVDDGFLDIWTYNAELLVLLENGRTLDFHDLNVKNLYEKVKSQLKNNAKEIGNGCFEFYHKCDNECMESVRNGTYDYPKYSEESKLNREKIDGVKLESMGVYQILAIYSTVASSLVLLVSLGAISFWMCSNGSLQCRICI

SEQ ID NO: 178 - protein, 566 aa, Influenza A Virus
MKANLLVLLCALAAADADTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTTKGVTAACSHAGKSSFYRNLLWLTEKEGSYPKLKNSYVNKKGKEVLVLWGIHHPSNSKDQQNIYQNENAYVSVVTSNYNRRFTPEIAERPKVRDQAGRMNYYWTLLKPGDTIIFEANGNLIAPRYAFALSRGFGSGIITSNASMHECNTKCQTPLGAINSSLPFQNIHPVTIGECPKYVRSAKLRMVTGLRNIPSIQSRGLFGAIAGFIEGGWTGMIDGWYGYHHQNEQGSGYAADQKSTQNAINGITNKVNSVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKLNREKVDGVKLESMGIYQILAIYSTVASSLVLLVSLGAISFWMCSNGSLQCRICI

SEQ ID NO: 179 - protein, 566 aa, Influenza A Virus
MKAKLLILLCALSATDADTICIGYHANNSTDTVDTVLEKNVTVTHSVNLLEDSHNGKLCRLKGIAPLQLGKCNIAGWILGNPECESLLSKRSWSYIAETPNSENGTCYPGDFADYEELREQLSSVSSFERFEIFPKERSWPKHNITRGVTAACSHAGKSSFYKNLLWLTETNGSYPKLSKSYVNNKEKEVLVLWGVHHPSNIEDQKTLYRKENAYVSVVSSNYNRRFTPEIAERPKVRGQAGRMNYYWTLLEPGDTIIFEANGNLIAPWYAFALSRGFGSGIITSNASMDECDTKCQTPQGAINSSLPFQNIHPVTIGECPKYVKSTKLRMVTGLRNIPSIQSRGLFGAIAGFIEGGWTGMIDGWYGYHHQNEQGSGYAADQKSTQNAINGITNKVNSVIEKMNTQFTAVGKEFNKLEKRMENLNKKVDDGFLDIWTYNAELLVLLENERTLDFHDSNVKNLYEKVKNQLRNNAKEIGNGCFEFYHKCNNECMESVKNGTYDYPKYSEESKLNREKIDGVKLESMGVYQILAIYSTVASSLVLLVSLGAISFWMCSNGSLQCRICI

SEQ ID NO: 180 - protein, 566 aa, Influenza A Virus
MKAKLLVLLCALSATDADTICIGYHANNSTDTVDTVLEKNVTVTHSVNLLEDSHNGKLCRLKGIAPLQLGKCNIAGWILGNPECESLFSKKSWSYIAETPNSENGTCYPGYFADYEELREQLSSVSSFERFEIFPKERSWPKHNVTRGVTASCSHKGKSSFYRNLLWLTEKNGSYPNLSKSYVNNKEKEVLVLWGVHHPSNIEDQKTIYRKENAYVSVVSSNYNRRFTPEIAERPKVRGQAGRINYYWTLLEPGDTIIFEANGNLIAPWHAFALNRGFGSGIITSNASMDECDTKCQTPQGAINSSLPFQNIHPVTIGECPKYVRSTKLRMVTGLRNIPSIQSRGLFGAIAGFIEGGWTGMIDGWYGYHHQNEQGSGYAADQKSTQNAINGITNKVNSVIEKMNTQFTAVGKEFNKLEKRMENLNKKVDDGFLDIWTYNAELLVLLENERTLDFHDSNVKNLYEKVKSQLKNNAKEIGNGCFEFYHKCNNECMESVKNGTYDYPKYSEESKLNREKIDGVKLESMGVYQILAIYSTVASSLVLLVSLGAISFWMCSNGSLQCRICI

SEQ ID NO: 181 - protein, 565 aa, Influenza A Virus
MKVKLLVLLCTFTATYADTICIGYHANNSTDTVDTVLEKNVTVTHSVNLLEDSHNGKLCLLKGIAPLQLGNCSVAGWILGNPECELLISRESWSYIVEKPNPENGTCYPGHFADYEELREQLSSVSSFERFEIFPKESSWPNHTTTGVSASCSHNGESSFYKNLLWLTGKNGLYPNLSKSYANNKEKEVLVLWGVHHPPNIGDQRALYHKENAYVSVVSSHYSRKFTPEIAKRPKVRDQEGRINYYWTLLEPGDTIIFEANGNLIAPRYAFALSRGFGSGIINSNAPMDECDAKCQTPQGAINSSLPFQNVHPVTIGECPKYVRSAKLRMVTGLRNIPSIQSRGLFGAIAGFIEGGWTGMVDGWYGYHHQNEQGSGYAADQKSTQNAINGITNKVNSVIEKMNTQFTAVGKEFNKLERRMENLNKKVDDGFIDIWTYNAELLVLLENERTLDFHDSNVKNLYEKVKSQLKNNAKEIGNGCFEFYHKCNDECMESVKNGTYDYPKYSEESKLNREKIDGVKLESMGVYQILAIYSTVASSLVLLVSLGAISFWMCSNGSLQCRICI

SEQ ID NO: 182 - protein, 565 aa, Influenza A Virus
MKVKLLVLLCTFTATYADTICIGYHANNSTDTVDTVLEKNVTVTHSVNLLENSHNGKLCLLKGIAPLQLGNCSVAGWILGNPECELLISKESWSYIVEKPNPENGTCYPGHFADYEELREQLSSVSSFERFEIFPKESSWPNHTVTGVSASCSHNGESSFYRNLLWLTGKNGLYPNLSKSYANNKEKEVLVLWGVHHPPNIGDQKALYHTENAYVSVVSSHYSRKFTPEIAKRPKVRDQEGRINYYWTLLEPGDTIIFEANGNLIAPRYAFALSRGFGSGIINSNAPMDKCDAKCQTPQGAINSSLPFQNVHPVTIGECPKYVRSAKLRMVTGLRNIPSIQSRGLFGAIAGFIEGGWTGMVDGWYGYHHQNEQGSGYAADQKSTQNAINGITNKVNSVIEKMNTQFTAVGKEFNKLERRMENLNKKVDDGFIDIWTYNAELLVLLENERTLDFHDSNVKNLYEKVKSQLKNNAKEIGNGCFEFYHKCNDECMESVKNGTYDYPKYSEESKLNREKIDGVKLESMGVYQILAIYSTVASSLVLLVSLGAISFWMCSNGSLQCRICI

SEQ ID NO: 183 - protein, 566 aa, Influenza A Virus
MKAILVVLLYTFTTANADTLCIGYHANNSTDTVDTVLEKNVTVTHSVNLLEDKHNGKLCKLRGVAPLHLGKCNIAGWILGNPECESLSTASSWSYIVETSNSDNGTCYPGDFINYEELREQLSSVSSFERFEIFPKTSSWPNHDSNKGVTAACPHAGAKSFYKNLIWLVKKGNSYPKLNQSYINDKGKEVLVLWGIHHPSTTADQQSLYQNADAYVFVGTSRYSKKFKPEIATRPKVRDQEGRMNYYWTLVEPGDKITFEATGNLVVPRYAFTMERNAGSGIIISDTPVHDCNTTCQTPEGAINTSLPFQNIHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLESTRIYQILAIYSTVASSLVLVVSLGAISFWMCSNGSLQCRICI

SEQ ID NO: 184 - protein, 566 aa, Influenza A Virus
MKAILVVLLYTFTTANADTLCIGYHANNSTDTVDTVLEKNVTVTHSVNLLEDKHNGKLCKLRGVAPLHLGKCNIAGWILGNPECESLSTARSWSYIVETSNSDNGTCYPGDFINYEELREQLSSVSSFERFEIFPKTSSWPNHDSDKGVTAACPHAGAKSFYKNLIWLVKKGNSYPKLNQTYINDKGKEVLVLWGIHHPPTIAAQESLYQNADAYVFVGTSRYSKKFKPEIATRPKVRDQEGRMNYYWTLVEPGDKITFEATGNLVVPRYAFTMERDAGSGIIISDTPVHDCNTTCQTPEGAINTSLPFQNVHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLESTRIYQILAIYSTVASSLVLVVSLGAISFWMCSNGSLQCRICI

SEQ ID NO: 185 - protein, 131 aa, synthetic construct
TGTVTQNGNAKGPGSGRNMTGTKSGSTVLNSTNQETSLVQAQSSSTKINNPDTISEFVKIACKYVKQNNVPTITNNDNFDKLIIRTRRSTIIPNIGSLSSRIKGVVNSNRLIDIHVQNETLFVSKFRMRTS

SEQ ID NO: 186 - protein, 131 aa, synthetic construct
TGTVTQNGNAKGPGSGRNMTGTKSGSTVLNSTNQETSLVQAQSSSTKINNPDTISEFVKIACKYVKQNNVPTITNNDNFDKLIVRTRRSTIIPNIGSLSSRIKGVVNSNRLIDIHVQNETLFVSKFRMRTS

SEQ ID NO: 187 - protein, 131 aa, synthetic construct
NGTVTQNGSAKGPDSGRNMTGYKSGSTVLNSTNQETSLVQAQSSSTKINNPGTISEFVKIACKYVKQNNVPTITNNDNFDKLIVRTKRSTIIPNIGSLSSRIKGIVNSNRLIDIHGQNETLFVSKFRMRTS

SEQ ID NO: 188 - protein, 131 aa, synthetic construct
NGNVTQNGNAKGPDIGRNNTGYKSGSTVQNSTDKETDLVQAQSSSTKINNPGTSSEFVKIACKYVKQNNVPTITNNDNSDKLIVKTKRSTIIPNVGSLSSRIKGIVNSNRLINIHGQNEKLFVSKFRMRTS

SEQ ID NO: 189 - protein, 131 aa, synthetic construct
NGNVTQNGYAKGPDNSRNMTGYKSESTVLNSTDKETNLVQAQSSSTRIDSPGTSSEFVKIACKYVKQNNVPTITNNDNFDKLIVRTKRSTIIPNVGSLSSGIKGILNSNRLKNIHGQNEKLFVSKFRIRTS

SEQ ID NO: 190 - protein, 131 aa, synthetic construct
NGNVTQSGYAKGSDKSRNMTGYESESKVLNSTDKETNLVRAQSSSTRIDSPGTSSEFVKIACKYVKQNNVPTITNNGNFDKLIVRTKRSTIIPNIGSLSSGIKGILNSNRLKNIHGQNEKLFVSKFRIRTS

SEQ ID NO: 191 - protein, 131 aa, synthetic construct
NDNVTQSGCAKGSVNSRNMIGHESEQKALNSTDRETNLVRAQSSSTRIDSPGTSSEFVKIACRYVKQNNVPTITNNGKFDKLIVRTKRSTVIPNIGSLSSRIKGILNSIRLKNIHGQNEKLFVSKYRIRTS

SEQ ID NO: 192 - protein, 131 aa, synthetic construct
NDNVAQSEYAKGSVKSRNMTGHKSEYKALNSTDRETNLVRAQSSSTRIDSPGTSSEFVRIACRYVKQNNVPTITNNGKFDKLIVRTKRSTVIPNIGSLSSRIKGILNSTRLKNIHGQNKELFVSKYRIRTS

SEQ ID NO: 193 - protein, 131 aa, synthetic construct
NDNVAQDGYAKGSVNSRNMTGHKSEYKALNSTDRDTSLVRAQSSSTRIDSPGTSSEFVRIACRYVKQNNVPTITNNGKFDKLIVRTKRSTVTPNIGSQSSRIKGILNSTRLKNIHGQNKELFVSKYRIRNS

SEQ ID NO: 194 - protein, 131 aa, synthetic construct
  SITE 2: note = misc_feature - Xaa can be any naturally occurring amino acid
NXNVAQDTYAKGSVKSRNMTGHKLEYKALNSTDSDTSIVQAQSSSTRIDSPGNNSEFVRIACRYVKQNNVPTTTNNDKFDKLIVRTKRSTVIPNIGSISSRIKGILNSTRLKNIHGQNKELFVSKYRIRSS

SEQ ID NO: 195 - protein, 131 aa, synthetic construct
NSNVAQNTYAKSSIKSRNMTGHQLKYKALNSTDSDTSIAQAQSSSTRIDSPGKNSEFVRIACRYVKQNNVPTNTNNDKFDKLIVRTKRSTVIPNIGSISSRIKGILNSKRLENIHGQNKELFVSKYRIRSS

SEQ ID NO: 196 - protein, 131 aa, synthetic construct
NSNVAQNTSSKRSIKSRNMTGHQLKYRALNSTDSDTSLTQAQSSSTRIDSPGKNSEFVRIACRYVKQNNVPTNTNNDKFDKLIVRTKRSTVIPNIGSISSRIKGILNSTQLENIHGQNKELFVSKYRIRSS

SEQ ID NO: 197 - protein, 131 aa, synthetic construct
NSNVAQNTSAKRSIKSRNMTGHQLNYKALNNTDSDISIAQAQSSSTRIDSPGKNSEFVRIACRYVKQNNVPTNTNNEKFDKLIVRTKRSTVIPNIGSVSSGIKGILNSTQLENIHGQNKELFVSTYRIRSS

SEQ ID NO: 198 - protein, 131 aa, synthetic construct
NSNVTQNTSAKRSNKSRNMTGTHLKYKALNGTDSDISLAQAQSSSTGIDSPGKNSEFVRIACRYVKQNNVPTNTNNEKFDKLIVRTKRSTVIPNIGSVSSRIKGILNSTQLENIQGQNKKLFVSKYRIRSS

SEQ ID NO: 199 - protein, 131 aa, synthetic construct
NSNVTQNTSAKRSNKSRNMTGTHLKYKALNGTDSDISLAQAQSSSTGIDSPGKNSEFVRIACRYVKQNNVPTTTNNEKFDKLIVRTKRSTVIPNIGSISSRIKGILNSTQLENIQGQNKKLFVSKYRIRSS

SEQ ID NO: 200 - protein, 131 aa, synthetic construct
NSNVTQNTSSKRSNNSRNMTGTHLKFKALNGTNNDISLTQAQSSSTGIDSPGKNSEFVRIACRYVKQNNVPTNTNNEKFDKLIVRTKRSTVIPNIGSIPSRIKGILNSTQLENIQGQNKKLFVSKYRIRSS

SEQ ID NO: 201 - protein, 131 aa, synthetic construct
DSNVTQNTSSKRSNNSRNMTGTHLKFKALNVTDNDIFLAQAQSSSTGIDSPGKNSEFVRIACRYVKQNNVPTNTNNEKFDKLIVRTKRSTVIPNIGSIPSRIKGILNSTQLENIQGQNKKLFVSKYRIRSS

SEQ ID NO: 202 - protein, 131 aa, synthetic construct
NSNVTQNTSAIRSNNSRNMTGTHLKFKALNGTDNDIFPAQAQSSSTEIDSPGKNSEFVRIACRYVKQNNVPTNTNNEKFDKLIVRTKRSTVIPNIGSIPSRIKGILNSTQLENIQGQNKKLFVSKYRIRSS

SEQ ID NO: 203 - protein, 131 aa, synthetic construct
NSNVTQNTSAIRSKNSRNMTGTHLNFKALNGTDKDIFLAQAQSSSTEIDSPGKNSEFVRIACRYVKQNNVPTNTNNEQFDKLIVRTKRSTVSPNIGSIPSRIKGILNSTQLKNIQGQNKKLFVSKYRIRSS

SEQ ID NO: 204 - protein, 131 aa, synthetic construct
NSNVTQNTSAIRSNNSRNMTGTRLNFKALNVTDKEIFLAQSQNSSIEIDSPGKNSEFVRIACRYVKQSNVPTNTNNEQFDKLIVRTKRSAVIPNIGYIPSRIKGILNSTQLENIQGQNKKLFVSKYRIRSS

SEQ ID NO: 205 - protein, 131 aa, synthetic construct
NSNVTQNTSAIRSNNSRNMNGTHLNFKALNVTDKDIFLAQPQNSSIEIDSPGKKSEFVRIACRYVKQSNVPTNTNNEQFDKLIVRTKRSAVIPNIGFIPSRIKGILNSTQLENIQGQNKKLFVSKYRIRSS

SEQ ID NO: 206 - protein, 131 aa, synthetic construct
  SITE 97: note = misc_feature - Xaa can be any naturally occurring amino acid
NSNVTQNTSSRGSNSSRNMAGTHLNSKALNVTDKDIFLAQSQNSSIEIDSPGKKSEFVRIACRYVKQSNVPTNTNNEQFDKLIVRTKRSAVIPNIGXIPSRIKGILNSTQLENIQGQNKKLFVSKYRIRSS

SEQ ID NO: 207 - protein, 131 aa, synthetic construct
  SITE 107: note = misc_feature - Xaa can be any naturally occurring amino acid
NSNVTQNTSSIGSKSSRNMAGTHLNSKALNGTDKNISLAQSQNSSIEIDSPGKKSEFVRIACRYVKQSNVPTNTNNEQFDKLIVRTKRSAVIPNIGSIPSRIKGILXSTQLENIQGQNKKLFVNKYRIRSS

SEQ ID NO: 208 - protein, 131 aa, synthetic construct
NSNVTQNTSAIRSSSSRNMTGTHLNYKALNGTDKDIFPAQSQNSSIEIDSPGKKSEFVRIACRYVKHSSVPTNTNNEQFDKLIVRTKRSAVIPNIGSIPSRIKGILNSTQLENIQGQNKKLFVSKYRIRSS

SEQ ID NO: 209 - protein, 131 aa, synthetic construct
NSNVTQNTSAIGSSSSRNMTGTHLNYKALNGTDKDIFPAQSQNSSIEIDSPGKKSEFVRIACRYVKHSNVPTKTNKEQFDKLIVRTKRSAVIPNIGSIPSRIKGILNSTQLENIQGQNKKLFVSKYRIRSS

SEQ ID NO: 210 - protein, 131 aa, synthetic construct
NSNVTQNTSSIGSKSSRNMAGTHLNSKALNGTDKDISLAQSQNSSIEIDSPGKKSEFVRIACRYVKQSNVPTNTNNEQFDKLIVRTKRSAVIPNIGSIPSRIKGILNSTQLENIQGQNKKLFVNKYRIRSS

SEQ ID NO: 211 - protein, 131 aa, synthetic construct; identical to SEQ ID NO: 210

SEQ ID NO: 212 - protein, 131 aa, synthetic construct
NSNVTQNKFSIGSSSSRNMAGTHLNYIALNVTDKDISLAQSQNSSIEIDSPGKKSEFVRIACRYVKQSNVPTKTNKEQFDKLIVRTKRSAVIPNIGSIPSRIKGILNSTQLGNIQGQNKKLFVSRYRIRSS

SEQ ID NO: 213 - protein, 131 aa, synthetic construct
NSNVTQNKFSIGSSSSRNMAGTHLNYTALNGTDKDISLAQSQNSSIEIDSPGKKSEFVRIACRYVKQSNVPTKTNKEQFDKLIVRTKRSAVIPNIGSIPSRIKGILNSTQLGNIQGQNKKLFVSRYRIRSS

SEQ ID NO: 214 - protein, 131 aa, synthetic construct
  SITE 97: note = misc_feature - Xaa can be any naturally occurring amino acid
NSNVKQNTSAIGSSSSRNMTGTHLNYKALNRTDKDISLAQPQNSSIEIDSPGKKSEFVRIACRYVKQSNVPTKTNNEQFDKLIVRTKRSAVIPNIGXIPSRIKGILNSTQLGNIQGQNKELFVSRNRIRSS

SEQ ID NO: 215 - protein, 131 aa, synthetic construct; identical to SEQ ID NO: 186

SEQ ID NO: 216 - protein, 131 aa, synthetic construct; identical to SEQ ID NO: 186

SEQ ID NO: 217 - protein, 131 aa, synthetic construct
TGTVTQNGNAKGPGSGRNMTGTKSGSTVLNSTNQETSLVQAQSSSTKINNPDTISEFVKIACKYVKQNNVPTITNNDNFDKLIVRTRRSTIIPNIGSLSSRIKGVVNSNRLINIHVQDETLFVSKFRMRTS

SEQ ID NO: 218 - protein, 131 aa, synthetic construct
TGTVTQNGNAKGPGSGRNMTGTKSGSTVLNSTNQETSLVQAQSSSTKINNPDTISEFVKIACKYVKQNNVPTITNNDNFDKLIVRTRRSTIIPNIGSLSSRIKGVVNSNRLIDIHGQNETLFVSKFRMRTS

SEQ ID NO: 219 - protein, 131 aa, synthetic construct
NGTVTQNGNAKGPDSGRNMTGYKSGSTVLNSTDQETSLVQAQSSSTKINNPGTISEFVKIACKYVKQNNVPTITNNDNFDKLIVRTKRSTIIPNIGSLSSRIKGIVNSNRLIDIHGQNETLFVSKFRMRTS

SEQ ID NO: 220 - protein, 131 aa, synthetic construct; identical to SEQ ID NO: 219

SEQ ID NO: 221 - protein, 131 aa, synthetic construct
NGNVTQNGNAKGPDSGRNMTGYKSGSTVLNSTDQETNLVQAQSSSTKINNPGTISEFVKIACKYVKQNNVPTITNNDNFDKLIVRTKRSTIIPNIGSLSSRIKGIVNSNRLINIHGQNEKLFVSKFRMRTS

SEQ ID NO: 222 - protein, 131 aa, synthetic construct
NGNVTQNGNAKGPDSGRNMTGYKSGSTVQNSTDQETNLVQAQSSSTKINNPGTSSEFVKIACKYVKQNNVPTITNNDNFDKLIVRTKRSTIIPNVGSLSSRIKGIVNSNRLINIHGQNEKLFVSKFRMRTS

SEQ ID NO: 223 - protein, 131 aa, synthetic construct
NGNVTQNGSAKGPDNGRNMTGYKSGSTVQNSTDKETDLVQAQSSSTKIDNPGTSSEFVKIACKYVKQNNVPTITNNDNSDKLIVKTKRSTVIPNVGSLSSRVKGIVNSNRLINIHGQNEKLFVSKFRMRTS

SEQ ID NO: 224 - protein, 131 aa, synthetic construct
  SITE 9: note = misc_feature - Xaa can be any naturally occurring amino acid
NGNVTQNGXAKGPDNGRNMTGYKSGSTVQNSTDKETDLVQAQSSSTKIDNPGTSSEFVKIACKYVKQNNVPTITNNDNFDKLIVRTKRSTIIPNVGSLSSRIKGIVNSNRLKNIHGQNEKLFVSKFRMRTS

SEQ ID NO: 225 - protein, 131 aa, synthetic construct
NGNVTQNGYAKGPDNSRNMTGYKSESTVLNSTDKETNLVQAQSSSTRIDNPGTSSEFVKIACKYVKQNNVPTFTNNDNFDKLIVRTKRSTIIPNVGSLSSRIKGVLNSNRLKNIHGQNEKLFVSKFRIRTS

SEQ ID NO: 226 - protein, 131 aa, synthetic construct; identical to SEQ ID NO: 190

SEQ ID NO: 227 - protein, 131 aa, synthetic construct
NGNVTQSGYAKGSDNSRNMTGYESESKVLNSTDKETNLVRAQSSSTRIDSPGTSSEFVKIACKYVKQNNVPTITNNGNFDKLIVRTKRSTIIPNIGSLSSRIKGILNSNRLKNIHGQNEKLFVSKFRIRTS

SEQ ID NO: 228 - protein, 131 aa, synthetic construct
NSNVTQSGYAKGSDNSRNMTGYESESKVLNSTDKETNLVRAQSSSTRIDSPGTSSEFVKIACKYVKQNNVPTITNNGNFDKLIVRTKRSTIIPNIGSLSSRIKGILNSNRLKNVHGQNEKLFVSKFRIRTS

SEQ ID NO: 229 - protein, 131 aa, synthetic construct
NGNVTQSGYAKGSDNSRNMTGYESESKVLNSTDKETNLVRAQSSSTRIDSPGTSSEFVKIACRYVKQNNVPTITNNGKFDKLIVRTKRSTVIPNIGSLSSRIKGILNSTRLKNIHGQNEKLFVSKFRIRTS

SEQ ID NO: 230 - protein, 131 aa, synthetic construct
NGNVTQSGYAKGSVNSRNMTGYESESKVLNSTDKETNLVRAQSSSTRIDSPGTSSEFVKIACRYVKQNNVPTITNNGKFDKLIVRTKRSTVIPNIGSLSSRIKGILNSTRLKNIHGQNEKLFVSKFRIRTS

SEQ ID NO: 231 - protein, 131 aa, synthetic construct
NGNVTQSGYTKGSVNSRNMTGYESESKVLNSTDKETNLVRAQSSSTRIDSPGTSSEFVKIACRYVKQNNVPTITNNGKFDKLIVKTKRSTVIPNIGSLSSRIKGILNSTRLKNIHGQNEKLFVSKFRIRTS

SEQ ID NO: 232 - protein, 131 aa, synthetic construct
NGNVTQSGYAKGSVNSRNMTGYESESKALNSTDKETNLVRAQSSSTRIDSPGTSSEFVKIACRYVKQNNVPTITNNGKFDKLIVRTKRSTVIPNIGSLSSRIKGILNSTRLKNIHGQNEKLFVSKFRIRTS

SEQ ID NO: 233 - protein, 131 aa, synthetic construct
NGNVTQSGYAKGSVNSRNMTGYESEYKALNSTDKETNLVRAQSSSTRIDSPGTSSEFVKIACRYVKQNNVPTITNNGKFDKLIVRTKRSTVIPNIGSLSSRIKGILNSTRLKNIHGQNEKLFVSKFRIRTS

SEQ ID NO: 234 - protein, 131 aa, synthetic construct
NDNVTQSGYAKGSVNSRNMTGHESEYKALNITDRETNLVRAQSSSTRIDSPGTSSEFVKIACRYVKQNNVPTITNNGKFDKLIVRTKRSTVIPNIGSLSSRIKGILNSTRLKNIHGQNEKLFVSKYRIRTS

SEQ ID NO: 235 - protein, 131 aa, synthetic construct
NDNVTQSGYAKGSVNSRNMTGHESEYKALNSTDRETNLVRAQSSSTRIDSPGTSSEFVKIACRYVKQNNVPTITNNGKFDKLIVRTKRSTVIPNIGSLSSRIKGILNSTRLKNIHGQNEKLFVSKYRIRTS

SEQ ID NO: 236 - protein, 131 aa, synthetic construct
NDNVAQSGYAKGSVNSRNMTGHESEYKALNSTDRETNLVRAQSSSTRIDSPGTSSEFVRIACRYVKQNNVPTITNNGKFDKLIVRTKRSTVIPNIGSLSSRIKGILNSTRLKNIHGQNKELFVSKYRIRTS

SEQ ID NO: 237 - protein, 131 aa, synthetic construct; identical to SEQ ID NO: 236

SEQ ID NO: 238 - protein, 131 aa, synthetic construct
NDNVAQSEYAKGSVKSRNMTGHESDYKALNSTDRETSLVRAQSSSTRIDSPGTSSEFVRIACRYVKQNNVPTITNNGKFDKLIVRTKRSTVIPNIGSLSSRIKGILNSTRLKNIHGQNKELFVSKYRIRTS

SEQ ID NO: 239 - protein, 131 aa, synthetic construct
NDNVAQSDYAKGSVKSRNMTGHESEYKALNSTDRETSLVRAQSSSTRIDSPGTSSEFVRIACRYVKQNNVPTITNNGKFDKLIVRTKRSTVIPNIGSLSSRIKGILNSTRLKNIHGQNKELFVSKYRIRTS

SEQ ID NO: 240 - protein, 131 aa, synthetic construct
NDNVAQDKYAKGSVNSRNMTGHKLEYKALNSTDSDTSLVRAQSSSTRIDSPGNSSEFVRIACRYVKQNNVPTITNNGKFDKLIVRTKRSTVIPNIGSLSSRIKGILNSTRLKNIHGQNKELFVSKYRIRNS

SEQ ID NO: 241 - protein, 131 aa, synthetic construct
NDNVAQDKYAKGSVNSRNMTGHKLEYKALNSTDSDTSLVRAQSSSTRIDSPGNSSEFVRIACRYVKQNNVPTITNNGKFDKLIVRTKRSTVIPNIGFLSSRIKGILNSTRLKNIHGQNKELFVSKYRIRNS

SEQ ID NO: 242 - protein, 131 aa, synthetic construct
NGNVAQDKYAKGSVNSRNMTGHKLEYKALNSTDSDTSLVQAQSSSTRIDSPGNNSEFVRIACRYVKQNNVPTTTNNDKFDKLIVRTKRSTVIPNIGSISSRIKGILNSTRLKNIHGQNKELFVSKYRIRNS

SEQ ID NO: 243 - protein, 131 aa, synthetic construct
NGNVAQDTYAKGSVKSRNMTGHKLEYKALNSTDSDTSLVQAQSSSTRIDSPGNNSEFVRIACRYVKQNNVPTTTNNDKFDKLIVRTKRSTVIPNIGSVSSRIKGILNSTRLKNIHGQNKELFVSKYRIRSS

SEQ ID NO: 244 - protein, 131 aa, synthetic construct
NGNVAQNTYAKRSVKSRNMTGHKLEYKALNSTDSDTSLVQAQSSSTRIDSPDNNSEFVRIACRYVKQNNVPTTTNNDKFDKLIVRTKRSTVIPNIGSVSSRIKGILNSTRLKNIHGQNKELFVSKYRIRSS

SEQ ID NO: 245 - protein, 131 aa, synthetic construct
NSNVAQNTYAKRSIKSRNMTGHQLKYKALNSTDSDTSLAQAQSSSTRIDSPGKNSEFVRIACRYVKQNNVPTNTNNDKFDKLIVRTKRSTVIPNIGSVSSRIKGILNSTRLENIHGQNKELFVSKYRIRSS

SEQ ID NO: 246 - protein, 131 aa, synthetic construct
NSNVAQNTSAKRSIKSRNMTGHQLKYKALNSTDSDISLAQAQSSSTRIDSPGKNSEFVRIACRYVKQNNVPTNTNNEKFDKLIVRTKRSTVIPNIGSVSSRIKGILNSTQLENIHGQNKELFVSKYRIRSS

SEQ ID NO: 247 - protein, 131 aa, synthetic construct
  SITE 14: note = misc_feature - Xaa can be any naturally occurring amino acid
  SITE 108: note = misc_feature - Xaa can be any naturally occurring amino acid
NSNVAQNTSAKRSXKSRNMTGHQLKYKALNSTDSDISLAQAQSSSTRIDSPGKNSEFVRIACRYVKQNNVPTNTNNEKFDKLIVRTKRSTVIPNIGSVSSRIKGILNXTQLENIHGQNKELFVSTYRIRSS

SEQ ID NO: 248 - protein, 131 aa, synthetic construct
  SITE 31: note = misc_feature - Xaa can be any naturally occurring amino acid
NSNVAQNTSAKRSNKSRNMTGHQLKYKALNXTDSDISLAQAQSSSTRIDSPGKNSEFVRIACRYVKQNNVPTNTNNEKFDKLIVRTKRSTVIPNIGSVSSRIKGILNSTQLENIHGQNKELFVSKYRIRSS

SEQ ID NO: 249 - protein, 131 aa, synthetic construct
NSNVAQNTSAKRSDKSRNMTGHQLKYKALNGTDSDISLAQAQSSSTRIDSPGKNSEFVRIACRYVKQNNVPTNTNNEKFDKLIVRTKRSTVIPNIGSVSSRIKGILNSTQLENIHGQNKELFVSKYRIRSS

SEQ ID NO: 250 - protein, 131 aa, synthetic construct; identical to SEQ ID NO: 198

SEQ ID NO: 251 - protein, 131 aa, synthetic construct
NSNVTQNTSAKRSNNSRNMTGTHLKFKALNGTDNDISLAQAQSSSTGIDSPGKNSEFVRIACRYVKQNNVPTNTNNEKFDKLIVRTKRSTVIPNIGSIPSRIKGILNSTQLENIQGQNKKLFVSKYRIRSS

SEQ ID NO: 252 - protein, 131 aa, synthetic construct
  SITE 37: note = misc_feature - Xaa can be any naturally occurring amino acid
NSNVTQNTSAKRSNNSRNMTGTHLKFKALNGTDNDIXLAQAQSSSTGIDSPGKNSEFVRIACRYVKQNNVPTNTNNEKFDKLIVRTKRSTVIPNIGSIPSRIKGILNSTQLENIQGQNKKLFVSKYRIRSS

SEQ ID NO: 253 - protein, 131 aa, synthetic construct
NSNVTQNTSAKRSNNSRNMTGTHLKFKALNGTDNDIFLAQAQSSSTGIDSPGKNSEFVRIACRYVKQNNVPTNTNNEKFDKLIVRTKRSTVIPNIGSIPSRIKGILNSTQLENIQGQNKKLFVSKYRIRSS

SEQ ID NO: 254 - protein, 131 aa, synthetic construct
NSNVTQNTSAIRSNNSRNMTGTHLKFKALNGTDNDIFLAQAQSSSTEIDSPGKNSEFVRIACRYVKQNNVPTNTNNEKFDKLIVRTKRSTVIPNIGSIPSRIKGILNSTQLENIQGQNKKLFVSKYRIRSS

SEQ ID NO: 255 - protein, 131 aa, synthetic construct
NSNVTQNTSAIRSNNSRNMTGTHLKFKALNGTDNDIFLAQAQSSSTEIDSPGKNSEFVRIACRYVKQNNVPTNTNNEQFDKLIVRTKRSTVIPNIGSIPSRIKGILNSTQLENIQGQNKKLFVSKYRIRSS

SEQ ID NO: 256 - protein, 131 aa, synthetic construct
NSNVTQNTSAIRSNNSRNMTGTHLNFKALNGTDKDIFLAQAQSSSTEIDSPGKNSEFVRIACRYVKQNNVPTNTNNEQFDKLIVRTKRSTVIPNIGSIPSRIKGILNSTQLENIQGQNKKLFVSKYRIRSS

SEQ ID NO: 257 - protein, 131 aa, synthetic construct
  SITE 56: note = misc_feature - Xaa can be any naturally occurring amino acid
NSNVTQNTSAIRSNNSRNMTGTHLNFKALNGTDKDIFLAQAQSSSTEINSPGKNSXFVRIACRYVKQNNVPTNTNNEQFDKLIVRTKRSAVIPNIGSIPSRIKGILNSTQLENIQGQNKKLFVSKHRIRSS

SEQ ID NO: 258 - protein, 131 aa, synthetic construct
NSNVTQNTSAIRSNNSRNMTGTHLNFKALNGTDKDIFLAQAQSSSTEIDSPGKNSEFVRIACRYVKQNNVPTNTNNEQFDKLIVRTKRSAVIPNIGSIPSRIKGILNSTQLENIQGQNKKLFVSKYRIRSS

SEQ ID NO: 259 - protein, 131 aa, synthetic construct
NSNVTQNTSAIRSNNSRNMTGTHLNFKALNGTDKDIFLAQSQNSSTEIDSPGKNSEFVRIACRYVKQSNVPTNTNNEQFDKLIVRTKRSAVIPNIGSIPSRIKGILNSTQLENIQGQNKKLFVSKYRIRSS

SEQ ID NO: 260 - protein, 131 aa, synthetic construct
  SITE 12: note = misc_feature - Xaa can be any naturally occurring amino acid
NSNVTQNTSAIXSNSSRNMTGTHLNFKALNGTDKDIFLAQSQNSSIEIDSPGKKSEFVRIACRYVKQSNVPTNTNNEQFDKLIVRTKRSAVIPNIGSIPSRIKGILNSTQLENIQGQNKKLFVSKYRIRSS

SEQ ID NO: 261 - protein, 131 aa, synthetic construct
  SITE 12: note = misc_feature - Xaa can be any naturally occurring amino acid
  SITE 67: note = misc_feature - Xaa can be any naturally occurring amino acid
NSNVTQNTSAIXSNSSRNMTGTHLNYKALNGTDKDIFLAQSQNSSIEIDSPGKKSEFVRIACRYVKXSNVPTNTNNEQFDKLIVRTKRSAVIPNIGSIPSRIKGILNSTQLENIQGQNKKLFVSKYRIRSS

SEQ ID NO: 262 - protein, 131 aa, synthetic construct
NSNVTQNTSAIRSSSSRNMTGTHLNYTALNGTDKDIFLAQSQNSSIEIDSPGKKSEFVRIACRYVKHSNVPTNTNNEQFDKLIVRTKRSAVIPNIGSIPSRIKGILNSTQLENIQGQNKKLFVSKYRIRSS

SEQ ID NO: 263 - protein, 131 aa, synthetic construct
  SITE 76: note = misc_feature - Xaa can be any naturally occurring amino acid
NSNVTQNTSAIRSSSSRNMTGTHLNYTALNGTDKDIFLAQSQNSSIEIDSPGKKSEFVRIACRYVKHSNVPTNTNXEQFDKLIVRTKRSAVIPNIGSIPSRIKGILNSTQLENIQGQNKKLFVSKYRIRSS

SEQ ID NO: 264 - protein, 131 aa, synthetic construct
  SITE 76: note = misc_feature - Xaa can be any naturally occurring amino acid
NSNVTQNTSAIRSSSSRNMTGTHLNYTALNGTDKDIFLAQSQNSSIEIDSPGKKSEFVRIACRYVKHSNVPTKTNXEQFDKLIVRTKRSAVIPNIGSIPSRIKGILNSTQLENIQGQNKKLFVSKYRIRSS

SEQ ID NO: 265 - protein, 131 aa, synthetic construct
NSNVKQNTSAIKSSSSRNMTGTHLNYTALNGTDKDIFLAQSQNSSIEIDSPGKKSEFVRIACRYVKHSNVPTNTNNEQFDKLIVRTKRSAVIPNIGSIPSRIKGILNSTQLENIQGQNKKLFVSKYRIQSS

SEQ ID NO: 266 - protein, 131 aa, synthetic construct
  SITE 26: note = misc_feature - Xaa can be any naturally occurring amino acid
NSNVTQNTSSIGSKSSRNMAGTHLNXKALNGTDKDISLAQSQNSSIEIDSPGKKSEFVRIACRYVKQSNVPTNTNNEQFDKLIVRTKRSAVIPNIGSIPSRIKGILNSTQLENIQGQNKKLFVNKYRIRSS

SEQ ID NO: 267 - protein, 131 aa, synthetic construct
NSNVTQNKSSIGSSSSRNMAGTHLNYTALNGTDKDISLAQSQNSSIEIDSPGKKSEFVRIACRYVKQSNVPTKTNKEQFDKLIVRTKRSAVIPNIGSIPSRIKGILNSTQLGNIQGQNKKLFVSRYRIRSS

SEQ ID NO: 268 - protein, 131 aa, Influenza A Virus
  SITE 9: note = misc_feature - Xaa can be any naturally occurring amino acid
  SITE 112: note = misc_feature - Xaa can be any naturally occurring amino acid
NGNVTQNGXAKGPDNGRNMTGYKSGSTVLNSTDKETNLVQAQSSSTKIDNPGTSSEFVKIACKYVKQNNVPTITNNDNFDKLIVRTKRSTIIPNIGSLSSRIKGIVNSNRLXNIHGQNEKLFVSKFRMRTS

SEQ ID NO: 269 - protein, 131 aa, Influenza A Virus
  REGION 7..8: note = misc_feature - Xaa can be any naturally occurring amino acid
NDNVAQXXYAKGSVNSRNMTGHKSEYKALNSTDRDTNLVRAQSSSTRIDSPGTSSEFVRIACRYVKQNNVPTITNNGKFDKLIVRTKRSTVIPNIGSLSSRIKGILNSTRLKNIHGQNKELFVSKYRIRTS

SEQ ID NO: 270 - protein, 131 aa, Influenza A Virus
  SITE 5: note = misc_feature - Xaa can be any naturally occurring amino acid
  SITE 23: note = misc_feature - Xaa can be any naturally occurring amino acid
  SITE 31: note = misc_feature - Xaa can be any naturally occurring amino acid
NSNVXQNTSAKRSNKSRNMTGHXLKYKALNXTDSDISLAQAQSSSTRIDSPGKNSEFVRIACRYVKQNNVPTNTNNEKFDKLIVRTKRSTVIPNIGSVSSRIKGILNSTQLENIHGQNKELFVSKYRIRSS

SEQ ID NO: 271 - protein, 131 aa, Influenza A Virus
NSNVTQNTSAIRSNNSRNMTGTHLNFKALNGTDKDIFLAQSQNSSIEIDSPGKKSEFVRIACRYVKQSNVPTNTNNEQFDKLIVRTKRSAVIPNIGSIPSRIKGILNSTQLENIQGQNKKLFVSKYRIRSS

SEQ ID NO: 272 - protein, 100 aa, Influenza A Virus
LNWLTKSGSTYPVLNVTMPNNDNFDKLYIWGIHHPSTNQEQTSLYVQASGRVTVSTRRSQQTIIPNIGSRPWVRGLSSRISIYWTIVKPGDVLVINSNGN

SEQ ID NO: 273 - protein, 100 aa, Influenza A Virus
LNWVTKSGSTYPVLNVTMPNNDNFDKLYIWGVHHPSTNQEQTSLYVQASGRVTVSTRRSQQTIIPNIGSRPWVRGLSSRISIYWTIVKPGDVLVINSNGN

SEQ ID NO: 274 - protein, 100 aa, Influenza A Virus
LNWLYKSGSTYPVLNVTMPNNDNFDKLYIWGVHHPSTNQEQTSLYVQASGRVTVSTKRSQQTIIPNIGSRPWVRGLSSRISIYWTIVKPGDILVINSNGN

SEQ ID NO: 275 - protein, 100 aa, Influenza A Virus
LNWLYKSESTYPVLNVTMPNNDNFDKLYIWGVHHPSTDKEQTNLYVQASGRVTVSTKRSQQTIIPNVGSRPWVRGLSSGISIYWTIVKPGDILLINSNGN

SEQ ID NO: 276 - protein, 100 aa, Influenza A Virus; identical to SEQ ID NO: 275

SEQ ID NO: 277 - protein, 100 aa, Influenza A Virus
LNWLYESESKYPVLNVTMPNNGNFDKLYIWGVHHPSTDKEQTNLYVRASGRVTVSTKRSQQTIIPNIGSRPWVRGLSSGISIYWTIVKPGDILLINSNGN

SEQ ID NO: 278 - protein, 100 aa, Influenza A Virus
LNWLHESEQKYPALNVTMPNNGKFDKLYIWGVHHPSTDREQTNLYVRASGRVTVSTKRSQQTVIPNIGSRPWVRGLSSRISIYWTIVKPGDILLINSIGN

SEQ ID NO: 279 - protein, 100 aa, Influenza A Virus
LNWLHKSEYKYPALNVTMPNNGKFDKLYIWGVHHPSTDREQTNLYVRASGRVTVSTKRSQQTVIPNIGSRPWVRGLSSRISIYWTIVKPGDILLINSTGN

SEQ ID NO: 280 - protein, 100 aa, Influenza A Virus
LNWLHKSEYKYPALNVTMPNNGKFDKLYIWGVHHPSTDRDQTSLYVRASGRVTVSTKRSQQTVTPNIGSRPWVRGQSSRISIYWTIVKPGDILLINSTGN

SEQ ID NO: 281 - protein, 100 aa, Influenza A Virus
LNWLHKLEYKYPALNVTMPNNDKFDKLYIWGVHHPSTDSDQTSIYVQASGRVTVSTKRSQQTVIPNIGSRPWVRGISSRISIYWTIVKPGDILLINSTGN

SEQ ID NO: 282 - protein, 100 aa, Influenza A Virus
LNWLHQLKYKYPALNVTMPNNDKFDKLYIWGVHHPSTDSDQTSIYAQASGRVTVSTKRSQQTVIPNIGSRPWVRGISSRISIHWTIVKPGDILLINSKGN

SEQ ID NO: 283 - protein, 100 aa, Influenza A Virus
LNWLHQLKYRYPALNVTMPNNDKFDKLYIWGVHHPSTDSDQTSLYTQASGRVTVSTKRSQQTVIPNIGSRPWVRGISSRISIYWTIVKPGDILLINSTGN

SEQ ID NO: 284 - protein, 100 aa, Influenza A Virus
LNWLHQLNYKYPALNVTMPNNEKFDKLYIWGVHHPNTDSDQISIYAQASGRVTVSTKRSQQTVIPNIGSRPWVRGVSSGISIYWTIVKPGDILLINSTGN

SEQ ID NO: 285 - protein, 100 aa, Influenza A Virus
LNWLTHLKYKYPALNVTMPNNEKFDKLYIWGVHHPGTDSDQISLYAQASGRITVSTKRSQQTVIPNIGSRPRIRDVSSRISIYWTIVKPGDILLINSTGN

SEQ ID NO: 286 - protein, 100 aa, Influenza A Virus
LNWLTHLKYKYPALNVTMPNNEKFDKLYIWGVHHPGTDSDQISLYAQASGRITVSTKRSQQTVIPNIGSRPRVRDISSRISIYWTIVKPGDILLINSTGN

SEQ ID NO: 287 - protein, 100 aa, Influenza A Virus
LNWLTHLKFKYPALNVTMPNNEKFDKLYIWGVHHPGTNNDQISLYTQASGRITVSTKRSQQTVIPNIGSRPRVRDIPSRISIYWTIVKPGDILLINSTGN

SEQ ID NO: 288 - protein, 100 aa, Influenza A Virus
LNWLTHLKFKYPALNVTMPNNEKFDKLYIWGVHHPVTDNDQIFLYAQASGRITVSTKRSQQTVIPNIGSRPRIRNIPSRISIYWTIVKPGDILLINSTGN

SEQ ID NO: 289 - protein, 100 aa, Influenza A Virus
LNWLTHLKFKYPALNVTMPNNEKFDKLYIWGVHHPGTDNDQIFPYAQASGRITVSTKRSQQTVIPNIGSRPRVRNIPSRISIYWTIVKPGDILLINSTGN

SEQ ID NO: 290 - protein, 100 aa, Influenza A Virus
LNWLTHLNFKYPALNVTMPNNEQFDKLYIWGVLHPGTDKDQIFLYAQASGRITVSTKRSQQTVSPNIGSRPRVRNIPSRISIYWTIVKPGDILLINSTGN

SEQ ID NO: 291 - protein, 100 aa, Influenza A Virus
LNWLTRLNFKYPALNVTMPNNEQFDKLYIWGVHHPVTDKEQIFLYAQSSGRITVSTKRSQQAVIPNIGYRPRIRNIPSRISIYWTIVKPGDILLINSTGN

SEQ ID NO: 292 - protein, 100 aa, Influenza A Virus
LNWLTHLNFKYPALNVTMPNNEQFDKLYIWGVHHPVTDKDQIFLYAQPSGRITVSTKRSQQAVIPNIGFRPRIRNIPSRISIYWTIVKPGDILLINSTGN

SEQ ID NO: 293 - protein, 100 aa, Influenza A Virus
  SITE 69: note = misc_feature - Xaa can be any naturally occurring amino acid
LNWLTHLNSKYPALNVTMPNNEQFDKLYIWGVHHPVTDKDQIFLYAQSSGRITVSTKRSQQAVIPNIGXRPRIRDIPSRISIYWTIVKPGDILLINSTGN

SEQ ID NO: 294 - protein, 100 aa, Influenza A Virus
  SITE 96: note = misc_feature - Xaa can be any naturally occurring amino acid
LNWLTHLNSKYPALNVTMPNNEQFDKLYIWGVHHPGTDKNQISLYAQSSGRITVSTKRSQQAVIPNIGSRPRIRDIPSRISIYWTIVKPGDILLIXSTGN

SEQ ID NO: 295 - protein, 100 aa, Influenza A Virus
LNWLTHLNYKYPALNVTMPNNEQFDKLYIWGVHHPGTDKDQIFPYAQSSGRITVSTKRSQQAVIPNIGSRPRIRDIPSRISIYWTIVKPGDILLINSTGN

SEQ ID NO: 296 - protein, 100 aa, Influenza A Virus
LNWLTHLNYKYPALNVTMPNKEQFDKLYIWGVHHPGTDKDQIFPYAQSSGRITVSTKRSQQAVIPNIGSRPRIRGIPSRISIYWTIVKPGDILLINSTGN

SEQ ID NO: 297 - protein, 100 aa, Influenza A Virus
LNWLTHLNSKYPALNVTMPNNEQFDKLYIWGVHHPGTDKDQISLYAQSSGRITVSTKRSQQAVIPNIGSRPRIRDIPSRISIYWTIVKPGDILLINSTGN

SEQ ID NO: 298 - protein, 100 aa, Influenza A Virus; identical to SEQ ID NO: 297

SEQ ID NO: 299 - protein, 100 aa, Influenza A Virus
LNWLTHLNYIYPALNVTMPNKEQFDKLYIWGVHHPVTDKDQISLYAQSSGRITVSTKRSQQAVIPNIGSRPRIRNIPSRISIYWTIVKPGDILLINSTGN

SEQ ID NO: 300 - protein, 100 aa, Influenza A Virus
LNWLTHLNYTYPALNVTMPNKEQFDKLYIWGVHHPGTDKDQISLYAQSSGRITVSTKRSQQAVIPNIGSRPRIRDIPSRISIYWTIVKPGDILLINSTGN

SEQ ID NO: 301 - protein, 100 aa, Influenza A Virus
  SITE 69: note = misc_feature - Xaa can be any naturally occurring amino acid
LNWLTHLNYKYPALNVTMPNNEQFDKLYIWGVHHPRTDKDQISLFAQPSGRITVSTKRSQQAVIPNIGXRPRIRDIPSRISIYWTIVKPGDILLINSTGN

SEQ ID NO: 302 - protein, 100 aa, Influenza A Virus
LNWLTKSGSTYPVLNVTMPNNDNFDKLYIWGVHHPSTNQEQTSLYVQASGRVTVSTRRSQQTIIPNIGSRPWVRGLSSRISIYWTIVKPGDVLVINSNGN

SEQ ID NO: 303 - protein, 100 aa, Influenza A Virus; identical to SEQ ID NO: 302

SEQ ID NO: 304 - protein, 100 aa, Influenza A Virus; identical to SEQ ID NO: 302

SEQ ID NO: 305 - protein, 100 aa, Influenza A Virus; identical to SEQ ID NO: 302

SEQ ID NO: 306 - protein, 100 aa, Influenza A Virus
LNWLYKSGSTYPVLNVTMPNNDNFDKLYIWGVHHPSTDQEQTSLYVQASGRVTVSTKRSQQTIIPNIGSRPWVRGLSSRISIYWTIVKPGDILVINSNGN

SEQ ID NO: 307 - protein, 100 aa, Influenza A Virus; identical to SEQ ID NO: 306

SEQ ID NO: 308 - protein, 100 aa, Influenza A Virus
LNWLYKSGSTYPVLNVTMPNNDNFDKLYIWGVHHPSTDQEQTNLYVQASGRVTVSTKRSQQTIIPNIGSRPWVRGLSSRISIYWTIVKPGDILVINSNGN

SEQ ID NO: 309 - protein, 100 aa, Influenza A Virus
LNWLYKSGSTYPVQNVTMPNNDNFDKLYIWGVHHPSTDQEQTNLYVQASGRVTVSTKRSQQTIIPNVGSRPWVRGLSSRISIYWTIVKPGDILVINSNGN

SEQ ID NO: 310 - protein, 100 aa, Influenza A Virus
LNWLYKSGSTYPVQNVTMPNNDNSDKLYIWGVHHPSTDKEQTDLYVQASGKVTVSTKRSQQTVIPNVGSRPWVRGLSSRVSIYWTIVKPGDILVINSNGN

SEQ ID NO: 311 - protein, 100 aa, Influenza A Virus
LNWLYKSGSTYPVQNVTMPNNDNFDKLYIWGVHHPSTDKEQTDLYVQASGRVTVSTKRSQQTIIPNVGSRPWVRGLSSRISIYWTIVKPGDILVINSNGN

SEQ ID NO: 312 - protein, 100 aa, Influenza A Virus
LNWLYKSESTYPVLNVTMPNNDNFDKLYIWGVHHPSTDKEQTNLYVQASGRVTVSTKRSQQTIIPNVGSRPWVRGLSSRISIYWTIVKPGDVLLINSNGN

SEQ ID NO: 313 - protein, 100 aa, Influenza A Virus; identical to SEQ ID NO: 277

SEQ ID NO: 314 - protein, 100 aa, Influenza A Virus
LNWLYESESKYPVLNVTMPNNGNFDKLYIWGVHHPSTDKEQTNLYVRASGRVTVSTKRSQQTIIPNIGSRPWVRGLSSRISIYWTIVKPGDILLINSNGN

SEQ ID NO: 315 - protein, 100 aa, Influenza A Virus; identical to SEQ ID NO: 314

SEQ ID NO: 316 - protein, 100 aa, Influenza A Virus
LNWLYESESKYPVLNVTMPNNGKFDKLYIWGVHHPSTDKEQTNLYVRASGRVTVSTKRSQQTVIPNIGSRPWVRGLSSRISIYWTIVKPGDILLINSTGN

SEQ ID NO: 317 - protein, 100 aa, Influenza A Virus; identical to SEQ ID NO: 316

SEQ ID NO: 318 - protein, 100 aa, Influenza A Virus
LNWLYESESKYPVLNVTMPNNGKFDKLYIWGVHHPSTDKEQTNLYVRASGKVTVSTKRSQQTVIPNIGSRPWVRGLSSRISIYWTIVKPGDILLINSTGN

SEQ ID NO: 319 - protein, 100 aa, Influenza A Virus
LNWLYESESKYPALNVTMPNNGKFDKLYIWGVHHPSTDKEQTNLYVRASGRVTVSTKRSQQTVIPNIGSRPWVRGLSSRISIYWTIVKPGDILLINSTGN

SEQ ID NO: 320 - protein, 100 aa, Influenza A Virus
LNWLYESEYKYPALNVTMPNNGKFDKLYIWGVHHPSTDKEQTNLYVRASGRVTVSTKRSQQTVIPNIGSRPWVRGLSSRISIYWTIVKPGDILLINSTGN

SEQ ID NO: 321 - protein, 100 aa, Influenza A Virus
LNWLHESEYKYPALNVTMPNNGKFDKLYIWGVHHPITDREQTNLYVRASGRVTVSTKRSQQTVIPNIGSRPWVRGLSSRISIYWTIVKPGDILLINSTGN

SEQ ID NO: 322 - protein, 100 aa, Influenza A Virus
LNWLHESEYKYPALNVTMPNNGKFDKLYIWGVHHPSTDREQTNLYVRASGRVTVSTKRSQQTVIPNIGSRPWVRGLSSRISIYWTIVKPGDILLINSTGN

SEQ ID NO: 323 - protein, 100 aa, Influenza A Virus; identical to SEQ ID NO: 322

SEQ ID NO: 324 - protein, 100 aa, Influenza A Virus; identical to SEQ ID NO: 322

SEQ ID NO: 325 - protein, 100 aa, Influenza A Virus
LNWLHESDYKYPALNVTMPNNGKFDKLYIWGVHHPSTDREQTSLYVRASGRVTVSTKRSQQTVIPNIGSRPWVRGLSSRISIYWTIVKPGDILLINSTGN

SEQ ID NO: 326 - protein, 100 aa, Influenza A Virus
LNWLHESEYKYPALNVTMPNNGKFDKLYIWGVHHPSTDREQTSLYVRASGRVTVSTKRSQQTVIPNIGSRPWVRGLSSRISIYWTIVKPGDILLINSTGN

SEQ ID NO: 327 - protein, 100 aa, Influenza A Virus
LNWLHKLEYKYPALNVTMPNNGKFDKLYIWGVHHPSTDSDQTSLYVRASGRVTVSTKRSQQTVIPNIGSRPWVRGLSSRISIYWTIVKPGDILLINSTGN

SEQ ID NO: 328 - protein, 100 aa, Influenza A Virus
LNWLHKLEYKYPALNVTMPNNGKFDKLYIWGVHHPSTDSDQTSLYVRASGRVTVSTKRSQQTVIPNIGFRPWVRGLSSRISIYWTIVKPGDILLINSTGN

SEQ ID NO: 329 - protein, 100 aa, Influenza A Virus
LNWLHKLEYKYPALNVTMPNNDKFDKLYIWGVHHPSTDSDQTSLYVQASGRVTVSTKRSQQTVIPNIGSRPWVRGISSRISIYWTIVKPGDILLINSTGN

SEQ ID NO: 330 - protein, 100 aa, Influenza A Virus
LNWLHKLEYKYPALNVTMPNNDKFDKLYIWGVHHPSTDSDQTSLYVQASGRVTVSTKRSQQTVIPNIGSRPWVRGVSSRISIYWTIVKPGDILLINSTGN

SEQ ID NO: 331 - protein, 100 aa, Influenza A Virus; identical to SEQ ID NO: 330

SEQ ID NO: 332 - protein, 100 aa, Influenza A Virus
LNWLHQLKYKYPALNVTMPNNDKFDKLYIWGVHHPSTDSDQTSLYAQASGRVTVSTKRSQQTVIPNIGSRPWVRGVSSRISIYWTIVKPGDILLINSTGN

SEQ ID NO: 333 - protein, 100 aa, Influenza A Virus
LNWLHQLKYKYPALNVTMPNNEKFDKLYIWGVHHPSTDSDQISLYAQASGRVTVSTKRSQQTVIPNIGSRPWVRGVSSRISIYWTIVKPGDILLINSTGN

SEQ ID NO: 334 - protein, 100 aa, Influenza A Virus
  SITE 49: note = misc_feature - Xaa can be any naturally occurring amino acid
  SITE 97: note = misc_feature - Xaa can be any naturally occurring amino acid
LNWLHQLKYKYPALNVTMPNNEKFDKLYIWGVHHPSTDSDQISLYAQAXGRVTVSTKRSQQTVIPNIGSRPWVRGVSSRISIYWTIVKPGDILLINXTGN

SEQ ID NO: 335 - protein, 100 aa, Influenza A Virus
  SITE 36: note = misc_feature - Xaa can be any naturally occurring amino acid
LNWLHQLKYKYPALNVTMPNNEKFDKLYIWGVHHPXTDSDQISLYAQASGRVTVSTKRSQQTVIPNIGSRPWVRGVSSRISIYWTIVKPGDILLINSTGN

SEQ ID NO: 336 - protein, 100 aa, Influenza A Virus
LNWLHQLKYKYPALNVTMPNNEKFDKLYIWGVHHPGTDSDQISLYAQASGRVTVSTKRSQQTVIPNIGSRPWVRGVSSRISIYWTIVKPGDILLINSTGN

SEQ ID NO: 337 - protein, 100 aa, Influenza A Virus
LNWLTHLKYKYPALNVTMPNNEKFDKLYIWGVHHPGTDSDQISLYAQASGRITVSTKRSQQTVIPNIGSRPRVRDVSSRISIYWTIVKPGDILLINSTGN

SEQ ID NO: 338 - protein, 100 aa, Influenza A Virus
LNWLTHLKFKYPALNVTMPNNEKFDKLYIWGVHHPGTDNDQISLYAQASGRITVSTKRSQQTVIPNIGSRPRVRDIPSRISIYWTIVKPGDILLINSTGN

SEQ ID NO: 339 - protein, 100 aa, Influenza A Virus
  SITE 43: note = misc_feature - Xaa can be any naturally occurring amino acid
LNWLTHLKFKYPALNVTMPNNEKFDKLYIWGVHHPGTDNDQIXLYAQASGRITVSTKRSQQTVIPNIGSRPRVRDIPSRISIYWTIVKPGDILLINSTGN

SEQ ID NO: 340 - protein, 100 aa, Influenza A Virus
LNWLTHLKFKYPALNVTMPNNEKFDKLYIWGVHHPGTDNDQIFLYAQASGRITVSTKRSQQTVIPNIGSRPRVRNIPSRISIYWTIVKPGDILLINSTGN

SEQ ID NO: 341 - protein, 100 aa, Influenza A Virus; identical to SEQ ID NO: 340

SEQ ID NO: 342 - protein, 100 aa, Influenza A Virus
LNWLTHLKFKYPALNVTMPNNEQFDKLYIWGVHHPGTDNDQIFLYAQASGRITVSTKRSQQTVIPNIGSRPRVRNIPSRISIYWTIVKPGDILLINSTGN

SEQ ID NO: 343 - protein, 100 aa, Influenza A Virus
LNWLTHLNFKYPALNVTMPNNEQFDKLYIWGVHHPGTDKDQIFLYAQASGRITVSTKRSQQTVIPNIGSRPRVRNIPSRISIYWTIVKPGDILLINSTGN

SEQ ID NO: 344 - protein, 100 aa, Influenza A Virus
LNWLTHLNFKYPALNVTMPNNEQFDKLYIWGVHHPGTDKDQIFLYAQASGRITVSTKRSQQAVIPNIGSRPRVRNIPSRISIYWTIVKPGDILLINSTGN

SEQ ID NO: 345 - protein, 100 aa, Influenza A Virus; identical to SEQ ID NO: 344

SEQ ID NO: 346 - protein, 100 aa, Influenza A Virus
LNWLTHLNFKYPALNVTMPNNEQFDKLYIWGVHHPGTDKDQIFLYAQSSGRITVSTKRSQQAVIPNIGSRPRIRNIPSRISIYWTIVKPGDILLINSTGN

SEQ ID NO: 347 - protein, 100 aa, Influenza A Virus; identical to SEQ ID NO: 346

SEQ ID NO: 348 - protein, 100 aa, Influenza A Virus
LNWLTHLNYKYPALNVTMPNNEQFDKLYIWGVHHPGTDKDQIFLYAQSSGRITVSTKRSQQAVIPNIGSRPRIRDIPSRISIYWTIVKPGDILLINSTGN

SEQ ID NO: 349 - protein, 100 aa, Influenza A Virus
LNWLTHLNYTYPALNVTMPNNEQFDKLYIWGVHHPGTDKDQIFLYAQSSGRITVSTKRSQQAVIPNIGSRPRIRDIPSRISIYWTIVKPGDILLINSTGN

SEQ ID NO: 350 - protein, 100 aa, Influenza A Virus
  SITE 21: note = misc_feature - Xaa can be any naturally occurring amino acid
LNWLTHLNYTYPALNVTMPNXEQFDKLYIWGVHHPGTDKDQIFLYAQSSGRITVSTKRSQQAVIPNIGSRPRIRDIPSRISIYWTIVKPGDILLINSTGN

SEQ ID NO: 351 - protein, 100 aa, Influenza A Virus; identical to SEQ ID NO: 350
  SITE 21: note = misc_feature - Xaa can be any naturally occurring amino acid

SEQ ID NO: 352 - protein, 100 aa, Influenza A Virus; identical to SEQ ID NO: 349

SEQ ID NO: 353 - protein, 100 aa, Influenza A Virus
  SITE 9: note = misc_feature - Xaa can be any naturally occurring amino acid
LNWLTHLNXKYPALNVTMPNNEQFDKLYIWGVHHPGTDKDQISLYAQSSGRITVSTKRSQQAVIPNIGSRPRIRDIPSRISIYWTIVKPGDILLINSTGN

SEQ ID NO: 354 - protein, 100 aa, Influenza A Virus; identical to SEQ ID NO: 300

SEQ ID NO: 355 - protein, 100 aa, Influenza A Virus
LNWLYKSGSTYPVLNVTMPNNDNFDKLYIWGVHHPSTDKEQTNLYVQASGRVTVSTKRSQQTIIPNIGSRPWVRGLSSRISIYWTIVKPGDILVINSNGN

SEQ ID NO: 356 - protein, 100 aa, Influenza A Virus
LNWLHKSEYKYPALNVTMPNNGKFDKLYIWGVHHPSTDRDQTSLYVRASGRVTVSTKRSQQTVIPNIGSRPWVRGLSSRISIYWTIVKPGDILLINSTGN

SEQ ID NO: 357 - protein, 100 aa, Influenza A Virus
LNWLTHLKYKYPALNVTMPNNEKFDKLYIWGVHHPGTDNDQISLYAQASGRITVSTKRSQQTVIPNIGSRPRVRDIPSRISIYWTIVKPGDILLINSTGN

SEQ ID NO: 358 - protein, 100 aa, Influenza A Virus; identical to SEQ ID NO: 348

SEQ ID NO: 359 - protein, 566 aa, Influenza A Virus
MKTIIALSYIFCQVLAQNLPGNDNSTATLCLGHHAVPNGTLVKTITNDQIEVTNATELVQSSSTGRICDSPHRILDGKNCTLIDALLGDPHCDGFQNEKWDLFVERSKAFSNCYPYDVPDYASLRSLVASSGTLEFINEGFNWTGVTQNGGSYACKRGPDNSFFSRLNWLYKSESTYPVLNVTMPNNDNFDKLYIWGVHHPSTDKEQTNLYVQASGRVTVSTKRSQQTIIPNVGSRPWVRGLSSGISIYWTIVKPGDILLINSNGNLIAPRGYFKIRTGKSSIMRSDAPIGTCSSECITPNGSIPNDKPFQNVNKITYGACPKYVKQNTLKLATGMRNVPEKQTRGIFGAKAGFIENGWEGMIDGWYGFRHQNSEGTGQAADLKSTQAAIDQINGKLNRVIEKTNEKFHQIEKEFSEVEGRIQDLEKYVEDTKIDLWSYNAELLVALENQHTIDLTDSEMNKLFEKTRRQLRENAEDMGNGCFKIYHKCDNACIGSIRNGTYDHDVYRDEALNNRFQIKGVELKSGYKDWILWISFAISCFLLCVVLLGFIMWACQKGNIRCNICI

SEQ ID NO: 360 - protein, 566 aa, Influenza A Virus
MKTIIALSYIFCLVFAQNLPGNDNSTATLCLGHHAVPNGTLVKTITNDQIEVTNATELVQSSSTGRICDSPHRILDGKNCTLIDALLGDPHCDGFQNEKWDLFVERSKAFSNCYPYDVPDYASLRSLVASSGTLEFINEGFNWTGVTQSGGSYACKRGSDKSFFSRLNWLYESESKYPVLNVTMPNNGNFDKLYIWGVHHPSTDKEQTNLYVRASGRVTVSTKRSQQTIIPNIGSRPWVRGLSSGISIYWTIVKPGDILLINSNGNLIAPRGYFKIRTGKSSIMRSDAPIGTCSSECITPNGSIPNDKPFQNVNKITYGACPKYVKQNTLKLATGMRNVPEKQTRGIFGAIAGFIENGWEGMVDGWYGFRHQNSEGTGQAADLKSTQAAIDQINGKLNRVIEKTNEKFHQIEKEFSEVEGRIQDLEKYVEDTKIDLWSYNAELLVALENQHTIDLTDSEMNKLFEKTRRQLRENAEDMGNGCFKIYHKCDNACIGSIRNGTYDHDVYRDEALNNRFQIKGVELKSGYKDWILWISFAISCFLLCVVLLGFIMWACQKGNIRCNICI

SEQ ID NO: 361 - protein, 566 aa, Influenza A Virus
MKTIIALSYILCLVFAQKLPGNDNSTATLCLGHHAVPNGTLVKTITNDQIEVTNATELVQSSSTGRICDSPHRILDGKNCTLIDALLGDPHCDGFQNKEWDLFVERSKAYSNCYPYDVPDYASLRSLVASSGTLEFINEDFNWTGVAQSGESYACKRGSVKSFFSRLNWLHKSEYKYPALNVTMPNNGKFDKLYIWGVHHPSTDREQTNLYVRASGRVTVSTKRSQQTVIPNIGSRPWVRGLSSRISIYWTIVKPGDILLINSTGNLIAPRGYFKIRTGKSSIMRSDAPIGTCSSECITPNGSIPNDKPFQNVNRITYGACPRYVKQNTLKLATGMRNVPEKQTRGIFGAIAGFIENGWEGMVDGWYGFRHQNSEGTGQAADLKSTQAAIDQINGKLNRLIEKTNEKFHQIEKEFSEVEGRIQDLEKYVEDTKIDLWSYNAELLVALENQHTIDLTDSEMNKLFEKTRKQLRENAEDMGNGCFKIYHKCDNACIGSIRNGTYDHDVYRDEALNNRFQIKGVELKSGYKDWILWISFAISCFLLCVVLLGFIMWACQKGNIRCNICI

SEQ ID NO: 362 - protein, 566 aa, Influenza A Virus
MKTIIALSYILCLVFAQKLPGNDNSTATLCLGHHAVPNGTLVKTITNDQIEVTNATELVQSSSTGRICDSPHRILDGKNCTLIDALLGDPHCDGFQNKEWDLFVERSKAYSNCYPYDVPDYASLRSLVASSGTLEFINEDFNWTGVAQDGGSYACKRGSVNSFFSRLNWLHKSEYKYPALNVTMPNNGKFDKLYIWGVHHPSTDRDQTSLYVRASGRVTVSTKRSQQTVTPNIGSRPWVRGQSSRISIYWTIVKPGDILLINSTGNLIAPRGYFKIRNGKSSIMRSDAPIGTCSSECITPNGSIPNDKPFQNVNRITYGACPRYVKQNTLKLATGMRNVPEKQTRGIFGAIAGFIENGWEGMVDGWYGFRHQNSEGTGQAADLKSTQAAIDQINGKLNRLIEKTNEKFHQIEKEFSEVEGRIQDLEKYVEDTKIDLWSYNAELLVALENQHTIDLTDSEMNKLFEKTRKQLRENAEDMGNGCFKIYHKCDNACIGSIRNGTYDHDVYRDEALNNRFQIKGVELKSGYKDWILWISFAISCFLLCVVLLGFIMWACQKGNIRCNICI

SEQ ID NO: 363 - protein, 566 aa, Influenza A Virus
MKTIIALSYILCLVFAQKLPGNDNSTATLCLGHHAVPNGTIVKTITNDQIEVTNATELVQSSSTGGICDSPHQILDGENCTLIDALLGDPQCDGFQNKKWDLFVERSKAYSNCYPYDVPDYASLRSLIASSGTLEFTNESFNWTGVTQNGTSSACKRRSNKSFFSRLNWLTHLKYKYPALNVTMPNNEKFDKLYIWGVHHPGTDSDQISLYAQASGRITVSTKRSQQTVIPNIGSRPRVRDISSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTRGIFGAIAGFIENGWEGMVDGWYGFRHQNSEGTGQAADLKSTQAAINQINGKLNKLIGKTNEKFHQIEKEFSEVEGRIQDLEKYVEDTKIDLWSYNAELLVALENQHTIDLTDSEMNKLFERTKKQLRENAEDMGNGCFKIYHKCDNACIGSIRNGTYDHDVYRDEALNNRFQIKGVELKSGYKDWILWISFAISCFLLCVVLLGFIMWACQKGNIRCNICI

SEQ ID NO: 364 - protein, 566 aa, Influenza A Virus
MKTIIALSYILCLVFAQKLPGNDNSTATLCLGHHAVPNGTIVKTITNDQIEVTNATELVQSSSTGGICDSPHQILDGENCTLIDALLGDPQCDGFQNKKWDLFVERSKAYSNCYPYDVPDYASLRSLVASSGTLEFNNESFNWTGVTQNGTSSACKRRSNKSFFSRLNWLTHLKYKYPALNVTMPNNEKFDKLYIWGVHHPGTDSDQISLYAQASGRITVSTKRSQQTVIPNIGSRPRIRDVSSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTRGIFGAIAGFIENGWEGMVDGWYGFRHQNSEGTGQAADLKSTQAAINQINGKLNRLIGKTNEKFHQIEKEFSEVEGRIQDLEKYVEDTKIDLWSYNAELLVALENQHTIDLTDSEMNKLFERTKKQLRENAEDMGNGCFKIYHKCDNACIGSIRNGTYDHDVYRDEALNNRFQIKGVELKSGYKDWILWISFAISCFLLCVALLGFIMWACQKGNIRCNICI

SEQ ID NO: 365 - protein, 566 aa, Influenza A Virus
MKTIIALSYILCLVFTQKIPGNDNSTATLCLGHHAVPNGTIVKTITNDRIEVTNATELVQNSSIGEICDSPHQILDGGNCTLIDALLGDPQCDGFQNKKWDLFVERSRAYSNCYPYDVPDYASLRSLVASSGTLEFKNESFNWAGVTQNGKSFSCIRGSSSSFFSRLNWLTHLNYIYPALNVTMPNKEQFDKLYIWGVHHPVTDKDQISLYAQSSGRITVSTKRSQQAVIPNIGSRPRIRNIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKSGYKDWILWISFAISCFLLCVALLGFIMWACQKGNIRCNICI

SEQ ID NO: 366 - protein, 566 aa, Influenza A Virus
  SITE 235: note = misc_feature - Xaa can be any naturally occurring amino acid
MKTIIALSYILCLVFAQKIPGNDNSTATLCLGHHAVPNGTIVKTITNDRIEVTNATELVQNSSIGEICDSPHQILDGGNCTLIDALLGDPQCDGFQNKEWDLFVERSRANSNCYPYDVPDYASLRSLVASSGTLEFKNESFNWTGVKQNGTSSACIRGSSSSFFSRLNWLTHLNYKYPALNVTMPNNEQFDKLYIWGVHHPRTDKDQISLFAQPSGRITVSTKRSQQAVIPNIGXRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKSGYKDWILWISFAMSCFLLCIALLGFIMWACQKGNIRCNICI

SEQ ID NO: 367 - protein, 72 aa, synthetic construct
MGNTPSANIRLSTSNVINTETAPGGPYKVNTETAPGGPYANGNGFFNDKTQMERLYGGFPNQTEDEGLKQSG

SEQ ID NO: 368 - protein, 72 aa, synthetic construct
TGTIPSAHIRLSTHNVINAETAPGGPYKVNAETAPGGPYTNGNGFFANEAQMVKLYGGFPNQAEDGGLPQSG

SEQ ID NO: 369 - protein, 72 aa, synthetic construct
MGTIPSANIRLSTRNVINAERAPGGPYIINAERAPGGPYTNGNGFFANEAQMVKLYGGFPNQTEDGGLPQSG

SEQ ID NO: 370 - protein, 72 aa, synthetic construct
MGTIPSANIRLSTHNVINAEAAPGGPYIVNAEAAPGGPYTNGNGFFANEIQMVKLYGGFPKQAEDGGLPQSG

SEQ ID NO: 371 - protein, 72 aa, synthetic construct
TGNIPSARIRLSNHNVINAEKAPGGPYKINAEKAPGGPYTNGNGFFASETQMAKLYGGFPNQTEDGGLPQSG

SEQ ID NO: 372 - protein, 72 aa, synthetic construct
TGNIPSAHIRLSTHNVINAEKAPGGPYKINAEKAPGGPYTNGNGFFANETQMAKLYGGFPNQTEDGGLPQSG

SEQ ID NO: 373 - protein, 72 aa, synthetic construct
TGNIPSAHIRLSTHNVINAENAPGGPYKINAENAPGGPYTNGNGFFANEIQMAKLYGGFPNQTEDGGLPQSG

SEQ ID NO: 374 - protein, 72 aa, synthetic construct
TGNIPSAHIRLSTHNVINAEKAPGGPYKINAEKAPGGPYTNGNGFFANEAQMAKLYGGFPNQTEDGGLPQSG

SEQ ID NO: 375 - protein, 72 aa, synthetic construct
TGKIPSAHIRLSTHNVINAENAPGGPYKINAENAPGGPYTNGNGFFANEAQMAKLYGGFPNQTEDGGLPQSG

SEQ ID NO: 376 - protein, 72 aa, synthetic construct
TGKIPSAHIRLSTHNVINAENAPGGPYKINAENAPGGPYTNGNGFFANETQMAKLYGGFPNQTEDGGLPQSG

SEQ ID NO: 377 - protein, 72 aa, synthetic construct
  SITE 48: note = misc_feature - Xaa can be any naturally occurring amino acid
TGKIPSAHVRLSTHNVINAEGAPGGPYKINAEGAPGGPYTNGNGFFAXETQMAKLYGGFPNQTEDGGLPQSG

SEQ ID NO: 378 - protein, 72 aa, synthetic construct
MGTIPSANIRLSTHNVINAERAPGGPYRLNAERAPGGPYTSRNGFFADKTQMKNLYGDFPNQTEDGGLPQSG

SEQ ID NO: 379 - protein, 72 aa, synthetic construct
VGTTPSANIRLSTQNVINAERAPGGPYRLNAERAPGGPYTSRDGFFADKTQMKNLYGGFPNQTEDGGLPQSG

SEQ ID NO: 380 - protein, 72 aa, synthetic construct
VGTTPSANIRLSTQNVINAEKAPGGPYRLNAEKAPGGPYTSRSGFFANKAQMKNLYGGFPDQTEDGGLPQSG

SEQ ID NO: 381 - protein, 72 aa, synthetic construct
VGTTPSANIRLSTQNVIDAEKAPGGPYRLDAEKAPGGPYTSKSGFFANKTQMKNLYGGFPDQTEDGGLPQSG

SEQ ID NO: 382 - protein, 72 aa, synthetic construct
VGTTPSANIRLSTQNVIDAENAPGGPYRLDAENAPGGPYTSKSGFFANKTQMKNLYGGFPAQTEDGGLPQSG

SEQ ID NO: 383 - protein, 72 aa, synthetic construct
VGTTPSANIRLSTQNVIDAEKAPGGPYRLDAEKAPGGPYTSKSGFFADKTQMKNLYGSFPDQTEDGGLPQSG

SEQ ID NO: 384 - protein, 72 aa, synthetic construct
VGTTPSANIRLSNQNVIDAERAPGGPYRLDAERAPGGPYTSKSGFFANKTQMKNLYGGFPDQTEDGGLPQSG

SEQ ID NO: 385 - protein, 72 aa, synthetic construct
VGTTPSANIRLSTQNVIDAEKAPGGPYRLDAEKAPGGPYTSKIGFFANKTQMKNLYGDFPDQTEDGGLPQSG

SEQ ID NO: 386 - protein, 72 aa, synthetic construct
VGTTPSAKIRLSTQNVIDAEKAPGGPYRLDAEKAPGGPYTSKIGFFANKTQMKSLYGDFPDQTEDGGLPQSG

SEQ ID NO: 387 - protein, 72 aa, synthetic construct; identical to SEQ ID NO: 367

SEQ ID NO: 388 - protein, 72 aa, synthetic construct
TGTIPSANIRLSTRNVINAETAPGGPYTVNAETAPGGPYTNGKGFFEDETQMVILYGGFPNQTEDEGLKQSG

SEQ ID NO: 389 - protein, 72 aa, synthetic construct
MGTIPSANIRLSARNVINAETAPGGPYIVNAETAPGGPYTNGNGFFANEIQMVKLYGGFPNQAEDEGLPQSG

SEQ ID NO: 390 - protein, 72 aa, synthetic construct
MGTIPSANIRLSARNVTNAETAPGGPYIVNAETAPGGPYTNGNGFFADETQMVKLYGGFPNQAEDEGLPQSG

SEQ ID NO: 391 - protein, 72 aa, synthetic construct
MGTIPSANIRLSTRNVINAERAPGGPYIINAERAPGGPYTNGNGFFATETQMVKLYGGFPNQTEDGGLPQSG

SEQ ID NO: 392 - protein, 72 aa, synthetic construct; identical to SEQ ID NO: 369

SEQ ID NO: 393 - protein, 72 aa, synthetic construct
MGTIPSANIKLSTRNVINAERAPGGPYIINAERAPGGPYTNGNGFFANAIQMVKLYGGFPNQTEDGGLPQSG

SEQ ID NO: 394 - protein, 72 aa, synthetic construct; identical to SEQ ID NO: 370

SEQ ID NO: 395 - protein, 72 aa, synthetic construct
MGTIPSAHIRLSTHNVINAETAPGGPYKVNAETAPGGPYTNGNGFFANEAQMVKLYGGFPNQAEDGGLPQSG

SEQ ID NO: 396 - protein, 72 aa, synthetic construct
MGTIPSANIRLSTHNVINAETAPGGPYKINAETAPGGPYTNGNGFFANETQMVKLYGGFPNQAEDGGLPQSG

SEQ ID NO: 397 - protein, 72 aa, synthetic construct
MGTIPSANIRLSTHNVINAETAPGGPYKINAETAPGGPYTNGNGFFADETQMVKLYGGFPNQAEDGGLPQSG

SEQ ID NO: 398 - protein, 72 aa, synthetic construct
TGTIPSAHIRLSTHNVINAEKAPGGPYKINAEKAPGGPYTNGNGFFANETQMAKLYGGFPNQAEDGGLPQSG

SEQ ID NO: 399 - protein, 72 aa, synthetic construct; identical to SEQ ID NO: 398

SEQ ID NO: 400 - protein, 72 aa, synthetic construct
TGTIPSAHIRLSTHNVINAEKAPGGPYKINAEKAPGGPYTNGNGFFANENQMAKLYGGFPNQAEDGGLPQSG

SEQ ID NO: 401 - protein, 72 aa, synthetic construct; identical to SEQ ID NO: 400

SEQ ID NO: 402 - protein, 72 aa, synthetic construct
  SITE 50: note = misc_feature - Xaa can be any naturally occurring amino acid
TGNIPSAHIRLSTHNVINAEKAPGGPYKINAEKAPGGPYTNGNGFFANEXQMAKLYGGFPNQTEDGGLPQSG

SEQ ID NO: 403 - protein, 72 aa, synthetic construct
  SITE 50: note = misc_feature - Xaa can be any naturally occurring amino acid
TGNIPSAHIRLSIHNVINAEKAPGGPYKINAEKAPGGPYTNGNGFFANEXQMAKLYGGFPNQTEDGGLPQSG

SEQ ID NO: 404 - protein, 72 aa, synthetic construct
TGNIPSAHIRLSIHNVINAEKAPGGPYKINAEKAPGGPYTNGNGFFANETQMAKLYGGFPNQTEDGGLPQSG

SEQ ID NO: 405 - protein, 72 aa, synthetic construct
  SITE 50: note = misc_feature - Xaa can be any naturally occurring amino acid
TGNIPSARIRLSNHNVINAEKAPGGPYKINAEKAPGGPYTNGNGFFANEXQMAKLYGGFPNQTEDGGLPQSG

SEQ ID NO: 406 - protein, 72 aa, synthetic construct; identical to SEQ ID NO: 372

SEQ ID NO: 407 - protein, 72 aa, synthetic construct; identical to SEQ ID NO: 372

SEQ ID NO: 408 - protein, 72 aa, synthetic construct
TGNIPSAHIRLSTHNVINAENAPGGPYKINAENAPGGPYTNGNGFFANETQMAKLYGGFPNQTEDGGLPQSG

SEQ ID NO: 409 - protein, 72 aa, synthetic construct; identical to SEQ ID NO: 372

SEQ ID NO: 410 - protein, 72 aa, synthetic construct; identical to SEQ ID NO: 408

SEQ ID NO: 411 - protein, 72 aa, synthetic construct; identical to SEQ ID NO: 408

SEQ ID NO: 412 - protein, 72 aa, synthetic construct; identical to SEQ ID NO: 376

SEQ ID NO: 413 - protein, 72 aa, synthetic construct; identical to SEQ ID NO: 376

SEQ ID NO: 414 - protein, 72 aa, synthetic construct; identical to SEQ ID NO: 376

SEQ ID NO: 415 - protein, 72 aa, synthetic construct; identical to SEQ ID NO: 376

SEQ ID NO: 416 - protein, 72 aa, synthetic construct; identical to SEQ ID NO: 376

SEQ ID NO: 417 - protein, 72 aa, synthetic construct; identical to SEQ ID NO: 376

SEQ ID NO: 418 - protein, 72 aa, synthetic construct
TGKIPSAHIRLSTHNVINAEDAPGGPYKINAEDAPGGPYTNGNGFFANETQMAKLYGGFPNQTEDGGLPQSG

SEQ ID NO: 419 - protein, 72 aa, synthetic construct
TGKIPSAHVRLSTHNVINAEDAPGGPYKINAEDAPGGPYTNGNGFFANETQMAKLYGGFPNQTEDGGLPQSG

SEQ ID NO: 420 - protein, 72 aa, synthetic construct; identical to SEQ ID NO: 419

SEQ ID NO: 421 - protein, 72 aa, synthetic construct; identical to SEQ ID NO: 419

SEQ ID NO: 422 - protein, 72 aa, synthetic construct
TGKIPSAHVRLSTHNVINAEGAPGGPYKINAEGAPGGPYTNGNGFFANETQMAKLYGGFPNQTEDGGLPQSG

SEQ ID NO: 423 - protein, 72 aa, synthetic construct
TGKIPSAHVRLSTHNVINAEDAPGRPYEINAEDAPGRPYTNGNGFFANETQMAKLYGGFPNQTEDGGLPQSG

SEQ ID NO: 424 - protein, 72 aa, synthetic construct
TGKIPSAHVRLSTHNVINAKDAPGRPYEINAKDAPGRPYTNGNGFFANETQMAKLYGGFPNQTEDGGLPQSG

SEQ ID NO: 425 - protein, 72 aa, synthetic construct; identical to SEQ ID NO: 378

SEQ ID NO: 426 - protein, 72 aa, synthetic construct
VGTTPSANIRLSTQNVINAERAPGGPYRLNAERAPGGPYTSRDGFFASKTQMKNLYGGFPNQTEDGGLPQSG

SEQ ID NO: 427 - protein, 72 aa, synthetic construct
VGTTPSANIRLSTQNVINAERAPGGPYRLNAERAPGGPYTSRDGFFANKTQMKNLYGGFPNQTEDGGLPQSG

SEQ ID NO: 428 - protein, 72 aa, synthetic construct
IGTIPSANIRLSTHNVINAERAPGGPYRLNAERAPGGPYTSKSGFFANKTQMKNLYGGFPNQTEDGGLPQSG

SEQ ID NO: 429 - protein, 72 aa, synthetic construct
VGTTPSAHIRLSTHNVINAEKAPGGPYRLNAEKAPGGPYTSRSGFFANKTQMKNLYGGFPNQTEDGGLPQSG

SEQ ID NO: 430 - protein, 72 aa, synthetic construct
IGTIPSAHIRLSTHNVINAERAPGGPYRLNAERAPGGPYTSRSGFFANKTQMKNLYGGFPNQTEDGGLPQSG

SEQ ID NO: 431 - protein, 72 aa, synthetic construct
  SITE 50: note = misc_feature - Xaa can be any naturally occurring amino acid
VGTTPSANIRLSTQNVINAEKAPGGPYRLNAEKAPGGPYTSRSGFFANKXQMKNLYGGFPDQTEDGGLPQSG

SEQ ID NO: 432 - protein, 72 aa, synthetic construct
VGTTPSAKIRLSTQNVINAEKAPGGPYRLNAEKAPGGPYTSRSGFFANKTQMKNLYGGFPDQTEDGGLPQSG

SEQ ID NO: 433 - protein, 72 aa, synthetic construct
VGTTPSANIRLSTQNVINAEKAPGGPYRLNAEKAPGGPYTSRSGFFANKTQMKNLYGGFPDQTEDGGLPQSG

SEQ ID NO: 434 - protein, 72 aa, synthetic construct
VGITPSAKIRLSTQNVINAEKAPGGPYRLNAEKAPGGPYTSRSGFFANKTQMKNLYGGFPDQTEDGGLPQSG

SEQ ID NO: 435 - protein, 72 aa, synthetic construct
VGTTPSANIRLSTQNVINAEKAPGGPYRLNAEKAPGGPYTSRSGFFANKTQMKNLYGDFPDQTEDGGLPQSG

SEQ ID NO: 436 - protein, 72 aa, synthetic construct; identical to SEQ ID NO: 434

SEQ ID NO: 437 - protein, 72 aa, synthetic construct
VGITPSAKIRLSTQNVINAEKAPGGPYRLNAEKAPGGPYTSKSGFFANKTQMKNLYGGFPDQTEDGGLPQSG

SEQ ID NO: 438 - protein, 72 aa, synthetic construct
VGITPSAKIRLSTQNVIDAEKAPGGPYRLDAEKAPGGPYTSKSGFFANKTQMKNLYGGFPDQTEDGGLPQSG

SEQ ID NO: 439 - protein, 72 aa, synthetic construct; identical to SEQ ID NO: 381

SEQ ID NO: 440 - protein, 72 aa, synthetic construct
VGTTPSANIRLSTQNVIDAENAPGGPYRLDAENAPGGPYTSKSGFFANKTQMKNLYGGFPDQTEDGGLPQSG

SEQ ID NO: 441 - protein, 72 aa, synthetic construct; identical to SEQ ID NO: 381

SEQ ID NO: 442 - protein, 72 aa, synthetic construct; identical to SEQ ID NO: 381

SEQ ID NO: 443 - protein, 72 aa, synthetic construct; identical to SEQ ID NO: 381

SEQ ID NO: 444 - protein, 72 aa, synthetic construct
  SITE 58: note = misc_feature - Xaa can be any naturally occurring amino acid
VGTTPSANIRLSTQNVIDAEKAPGGPYRLDAEKAPGGPYTSKSGFFANKTQMKNLYGXFPDQTEDGGLPQSG

SEQ ID NO: 445 - protein, 72 aa, synthetic construct
  SITE 43: note = misc_feature - Xaa can be any naturally occurring amino acid
VGTTPSANIRLSTQNVIDAEKAPGGPYRLDAEKAPGGPYTSKXGFFANKTQMKNLYGDFPDQTEDGGLPQSG

SEQ ID NO: 446 - protein, 72 aa, synthetic construct; identical to SEQ ID NO: 385

SEQ ID NO: 447 - protein, 72 aa, synthetic construct; identical to SEQ ID NO: 385

SEQ ID NO: 448 - protein, 72 aa, synthetic construct; identical to SEQ ID NO: 385

SEQ ID NO: 449 - protein, 72 aa, synthetic construct; identical to SEQ ID NO: 381

SEQ ID NO: 450 - protein, 72 aa, synthetic construct; identical to SEQ ID NO: 381

SEQ ID NO: 451 - protein, 72 aa, synthetic construct; identical to SEQ ID NO: 386

SEQ ID NO: 452 - protein, 72 aa, synthetic construct; identical to SEQ ID NO: 386

SEQ ID NO: 453 - protein, 72 aa, synthetic construct; identical to SEQ ID NO: 386

SEQ ID NO: 454 - protein, 72 aa, synthetic construct; identical to SEQ ID NO: 386

SEQ ID NO: 455 - protein, 72 aa, synthetic construct; identical to SEQ ID NO: 386

SEQ ID NO: 456 - protein, 72 aa, synthetic construct; identical to SEQ ID NO: 386

SEQ ID NO: 457 - protein, 72 aa, synthetic construct
VGTTPSAKIRLSTQNVIDAEKAPGGPYRLDAEKAPGGPYTSKIGFFANKTQMKSLYGDFPNQTEDGGLPQSG

SEQ ID NO: 458 - protein, 72 aa, Influenza B Virus
  SITE 3: note = misc_feature - Xaa can be any naturally occurring amino acid
  SITE 21: note = misc_feature - Xaa can be any naturally occurring amino acid
  SITE 33: note = misc_feature - Xaa can be any naturally occurring amino acid
TGXIPSAHIRLSTHNVINAEXAPGGPYKINAEXAPGGPYTNGNGFFANETQMAKLYGGFPNQTEDGGLPQSG

SEQ ID NO: 459 - protein, 72 aa, Influenza B Virus; identical to SEQ ID NO: 381

SEQ ID NO: 460 - protein, 100 aa, Influenza B Virus
GFFNTMAWVIPKDNNKTAINPVTVEVPYICSEGEDQITVWGFHSDDKTQMERLYGDSNPQKFTSSANGVTTHYVSQIGGFPNQTEDEGLKQSGRIVVDYM

SEQ ID NO: 461 - protein, 100 aa, Influenza B Virus
GFFATMAWAVPKNDNNKTATNPLTVEVPYICTEGEDQITVWGFHSDNEAQMVKLYGDSKPQKFTSSANGVTTHYVSQIGGFPNQAEDGGLPQSGRIVVDY

SEQ ID NO: 462 - protein, 100 aa, Influenza B Virus
GFFATMAWAVPKDNKTATNPLTVEVPYICTKGEDQITVWGFHSDNEAQMVKLYGDSKPQKFTSSANGVTTHYVSQIGGFPNQTEDGGLPQSGRIVVDYMV

SEQ ID NO: 463 - protein, 100 aa, Influenza B Virus
GFFATMAWAVPKNNNNKTATNPLTVEVPFICTEGEDQITVWGFHSDNEIQMVKLYGDSKPQKFTSSANGVTTHYVSQIGGFPKQAEDGGLPQSGRIVVDY

SEQ ID NO: 464 - protein, 100 aa, Influenza B Virus
GFFATMAWAVPKNENNKTATNSLTIEVPYICTEGEDQITVWGFHSDSETQMAKLYGDSKPQKFTSSANGVTTHYVSQIGGFPNQTEDGGLPQSGRIVVDY

SEQ ID NO: 465 - protein, 100 aa, Influenza B Virus
GFFATMAWAVPKNDNNKTATNSLTIEVPYICTEGEDQITVWGFHSDNETQMAKLYGDSKPQKFTSSANGVTTHYVSQIGGFPNQTEDGGLPQSGRIVVDY

SEQ ID NO: 466 - protein, 100 aa, Influenza B Virus
GFFATMAWAVPKNDNNKTATNSLTIEVPYICTEGEDQITVWGFHSDNEIQMAKLYGDSKPQKFTSSANGVTTHYVSQIGGFPNQTEDGGLPQSGRIVVDY

SEQ ID NO: 467 - protein, 100 aa, Influenza B Virus
GFFATMAWAVPKNDNNKTATNSLTIEVPYICTEGEDQITIWGFHSDNEAQMAKLYGDSKPQKFTSSANGVTTHYVSQIGGFPNQTEDGGLPQSGRIVVDY

SEQ ID NO: 468 - protein, 100 aa, Influenza B Virus
GFFATMAWAVPKNDKNKTATNPLTIEVPYICTEGEDQITVWGFHSDNEAQMAKLYGDSKPQKFTSSANGVTTHYVSQIGGFPNQTEDGGLPQSGRIVVDY

SEQ ID NO: 469 - protein, 100 aa, Influenza B Virus
GFFATMAWAVPKNDKNKTATNPLTIEVPYICTEGEDQITVWGFHSDNETQMAKLYGDSKPQKFTSSANGVTTHYVSQIGGFPNQTEDGGLPQSGRIVVDY

SEQ ID NO: 470 - protein, 100 aa, Influenza B Virus
  SITE 45: note = misc_feature - Xaa can be any naturally occurring amino acid
GFFATMAWAVPDKNKTATNPLTIEVPYVCTEGEDQITVWGFHSDXETQMAKLYGDSKPQKFTSSANGVTTHYVSQIGGFPNQTEDGGLPQSGRIVVDYMV

SEQ ID NO: 471 - protein, 100 aa, Influenza B Virus
GFFATMAWAVPRDNKTATNPLTVEVPYICTKGEDQITVWGFHSDDKTQMKNLYGDSNPQKFTSSANGVTTHYVSQIGDFPNQTEDGGLPQSGRIVVDYMV

SEQ ID NO: 472 - protein, 100 aa, Influenza B Virus
GFFATMAWAVPRDNKTATNPLTVEVPYICTKGEDQITVWGFHSDDKTQMKNLYGDSNPQKFTSSANGVTTHYVSQIGGFPNQTEDGGLPQSGRIVVDYMV

SEQ ID NO: 473 - protein, 100 aa, Influenza B Virus
GFFATMAWAVPRDDNKTATNPLTVEVPYVCTEGEDQITVWGFHSDNKAQMKNLYGDSNPQKFTSSANGVTTHYVSQIGGFPDQTEDGGLPQSGRIVVDYM

SEQ ID NO: 474 - protein, 100 aa, Influenza B Virus
GFFATMAWAVPKDNNKNATNPLTVEVPYICTEGEDQITVWGFHSDNKTQMKNLYGDSNPQKFTSSANGVTTHYVSQIGGFPDQTEDGGLPQSGRIVVDYM

SEQ ID NO: 475 - protein, 100 aa, Influenza B Virus
GFFATMAWAVPKDNNKNATNPLTVEVPYVCTEGEDQITVWGFHSDNKTQMKNLYGDSNPQKFTSSANGVTTHYVSQIGGFPAQTEDGGLPQSGRIVVDYM

SEQ ID NO: 476 - protein, 100 aa, Influenza B Virus
GFFATMAWAVPKDNNKNATNPLTVEVPYICTEGEDQITVWGFHSDDKTQMKNLYGDSNPQKFTSSANGVTTHYVSQIGSFPDQTEDGGLPQSGRIVVDYM

SEQ ID NO: 477 - protein, 100 aa, Influenza B Virus; identical to SEQ ID NO: 474

SEQ ID NO: 478 - protein, 100 aa, Influenza B Virus
GFFATMAWAVPKDNYKNATNPLTVEVPYICKEEEDQITVWGFHSDNKTQMKNLYGDSNPQKFTSSANGVTTHYVSQIGDFPDQTEDGGLPQSGRIVVDYM

SEQ ID NO: 479 - protein, 100 aa, Influenza B Virus
GFFATMAWAVPKDNYKNATNPLTVEVPYICTEGEDQITVWGFHSDNKTQMKSLYGDSNPQKFTSSANGVTTHYVSQIGDFPDQTEDGGLPQSGRIVVDYM

SEQ ID NO: 480 - protein, 100 aa, Influenza B Virus; identical to SEQ ID NO: 460

SEQ ID NO: 481 - protein, 100 aa, Influenza B Virus
GFFETMAWAVPKNKNKTATNPLTVEVPYICTKGEDQITVWGFHSDDETQMVILYGDSKPQKFTSSANGVTTHYVSQIGGFPNQTEDEGLKQSGRIVVDYI

SEQ ID NO: 482 - protein, 100 aa, Influenza B Virus
GFFATMAWAVPKNKTATNPLTVEVPYICAKGEDQITVWGFHSDNEIQMVKLYGDSKPQKFTSSANGVTTHYVSQIGGFPNQAEDEGLPQSGRIVVDYMVQ

SEQ ID NO: 483 - protein, 100 aa, Influenza B Virus
GFFATMAWAVPKNKTATNPLTVEVPYICTKGEDQITVWGFHSDDETQMVKLYGDSKPQKFTSSANGVTTHYVSQIGGFPNQAEDEGLPQSGRIVVDYMVQ

SEQ ID NO: 484 - protein, 100 aa, Influenza B Virus
GFFATMAWAVPKDNKTATNPLTVEVPYICTKGEDQITVWGFHSDTETQMVKLYGDSKPQKFTSSANGVTTHYVSQIGGFPNQTEDGGLPQSGRIVVDYMV

SEQ ID NO: 485 - protein, 100 aa, Influenza B Virus; identical to SEQ ID NO: 462

SEQ ID NO: 486 - protein, 100 aa, Influenza B Virus
GFFATMAWAVPKDNKTATNPLTVEVPYICTKGEDQITVWGFHSDNAIQMVKLYGDSKPQKFTSSANGVTTHYVSQIGGFPNQTEDGGLPQSGRIVVDYMV

SEQ ID NO: 487 - protein, 100 aa, Influenza B Virus; identical to SEQ ID NO: 463

SEQ ID NO: 488 - protein, 100 aa, Influenza B Virus; identical to SEQ ID NO: 461

SEQ ID NO: 489 - protein, 100 aa, Influenza B Virus
GFFATMAWAVPKNDNNKTATNPLTVEVPYICTEGEDQITVWGFHSDNETQMVKLYGDSKPQKFTSSANGVTAHYVSQIGGFPNQAEDGGLPQSGRIVVDY

SEQ ID NO: 490 - protein, 100 aa, Influenza B Virus
GFFATMAWAVPKNDNNKTATNPLTVEVPYICTEGEDQITVWGFHSDDETQMVKLYGDSKPQKFTSSANGVTAHYVSQIGGFPNQAEDGGLPQSGRIVVDY

SEQ ID NO: 491 - protein, 100 aa, Influenza B Virus
GFFATMAWAVPKNDNNKTATNSLTVEVPYICTEGEDQITVWGFHSDNETQMAKLYGDSKPQKFTSSANGVTTHYVSQIGGFPNQAEDGGLPQSGRIVVDY

SEQ ID NO: 492 - protein, 100 aa, Influenza B Virus; identical to SEQ ID NO: 491

SEQ ID NO: 493 - protein, 100 aa, Influenza B Virus
GFFATMAWAVPKNDNNKTATNSLTVEVPYICTEGEDQITVWGFHSDNENQMAKLYGDSKPQKFTSSANGVTTHYVSQIGGFPNQAEDGGLPQSGRIVVDY

SEQ ID NO: 494 - protein, 100 aa, Influenza B Virus; identical to SEQ ID NO: 493

SEQ ID NO: 495 - protein, 100 aa, Influenza B Virus
  SITE 49: note = misc_feature - Xaa can be any naturally occurring amino acid
GFFATMAWAVPKNDNNKTATNSLTIEVPYICTEGEDQITVWGFHSDNEXQMAKLYGDSKPQKFTSSANGVTTHYVSQIGGFPNQTEDGGLPQSGRIVVDY

SEQ ID NO: 496 - protein, 100 aa, Influenza B Virus; identical to SEQ ID NO: 495
  SITE 49: note = misc_feature - Xaa can be any naturally occurring amino acid

SEQ ID NO: 497 - protein, 100 aa, Influenza B Virus; identical to SEQ ID NO: 465

SEQ ID NO: 498 - protein, 100 aa, Influenza B Virus
  SITE 49: note = misc_feature - Xaa can be any naturally occurring amino acid
GFFATMAWAVPKNENNKTATNSLTIEVPYICTEGEDQITVWGFHSDNEXQMAKLYGDSKPQKFTSSANGVTTHYVSQIGGFPNQTEDGGLPQSGRIVVDY

SEQ ID NO: 499 - protein, 100 aa, Influenza B Virus; identical to SEQ ID NO: 465

SEQ ID NO: 500 - protein, 100 aa, Influenza B Virus; identical to SEQ ID NO: 465

SEQ ID NO: 501 - protein, 100 aa, Influenza B Virus; identical to SEQ ID NO: 465

SEQ ID NO: 502 - protein, 100 aa, Influenza B Virus
GFFATMAWAVPKNDNNKTATNSLTIEVPYICTEGEDQITIWGFHSDNETQMAKLYGDSKPQKFTSSANGVTTHYVSQIGGFPNQTEDGGLPQSGRIVVDY

SEQ ID NO: 503 - protein, 100 aa, Influenza B Virus; identical to SEQ ID NO: 465

SEQ ID NO: 504 - protein, 100 aa, Influenza B Virus; identical to SEQ ID NO: 465

SEQ ID NO: 505 - protein, 100 aa, Influenza B Virus; identical to SEQ ID NO: 469

SEQ ID NO: 506 - protein, 100 aa, Influenza B Virus; identical to SEQ ID NO: 469

SEQ ID NO: 507 - protein, 100 aa, Influenza B Virus; identical to SEQ ID NO: 469

SEQ ID NO: 508 - protein, 100 aa, Influenza B Virus; identical to SEQ ID NO: 469

SEQ ID NO: 509 - protein, 100 aa, Influenza B Virus; identical to SEQ ID NO: 469

SEQ ID NO: 510 - protein, 100 aa, Influenza B Virus; identical to SEQ ID NO: 469

SEQ ID NO: 511 - protein, 100 aa, Influenza B Virus; identical to SEQ ID NO: 469

SEQ ID NO: 512 - protein, 100 aa, Influenza B Virus; identical to SEQ ID NO: 469

SEQ ID NO: 513 - protein, 100 aa, Influenza B Virus; identical to SEQ ID NO: 469

SEQ ID NO: 514 - protein, 100 aa, Influenza B Virus; identical to SEQ ID NO: 469

SEQ ID NO: 515 - protein, 100 aa, Influenza B Virus
GFFATMAWAVPDKNKTATNPLTIEVPYVCTEGEDQITVWGFHSDNETQMAKLYGDSKPQKFTSSANGVTTHYVSQIGGFPNQTEDGGLPQSGRIVVDYMV

SEQ ID NO: 516 - protein, 100 aa, Influenza B Virus
GFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQ

SEQ ID NO: 517 - protein, 100 aa, Influenza B Virus; identical to SEQ ID NO: 516

SEQ ID NO: 518 - protein, 100 aa, Influenza B Virus; identical to SEQ ID NO: 471

SEQ ID NO: 519 - protein, 100 aa, Influenza B Virus
GFFATMAWAVPRDNKTATNPLTVEVPYICTKGEDQITVWGFHSDSKTQMKNLYGDSNPQKFTSSANGVTTHYVSQIGGFPNQTEDGGLPQSGRIVVDYMV

SEQ ID NO: 520 - protein, 100 aa, Influenza B Virus
GFFATMAWAVPRDNKTATNPLTVEVPYICTKGEDQITVWGFHSDNKTQMKNLYGDSNPQKFTSSANGVTTHYVSQIGGFPNQTEDGGLPQSGRIVVDYMV

SEQ ID NO: 521 - protein, 100 aa, Influenza B Virus; identical to SEQ ID NO: 520

SEQ ID NO: 522 - protein, 100 aa, Influenza B Virus; identical to SEQ ID NO: 520

SEQ ID NO: 523 - protein, 100 aa, Influenza B Virus; identical to SEQ ID NO: 520

SEQ ID NO: 524 - protein, 100 aa, Influenza B Virus
  SITE 29: note = misc_feature - Xaa can be any naturally occurring amino acid
  SITE 48: note = misc_feature - Xaa can be any naturally occurring amino acid
GFFATMAWAVPRDNNKTATNPLTVEVPYXCTKGEDQITVWGFHSDNKXQMKNLYGDSNPQKFTSSANGVTTHYVSQIGGFPDQTEDGGLPQSGRIVVDYM

SEQ ID NO: 525 - protein, 100 aa, Influenza B Virus
GFFATMAWAVPRDNNKTATNPLTVEVPYICTKEEDQITVWGFHSDNKTQMKNLYGDSNPQKFTSSANGVTTHYVSQIGGFPDQTEDGGLPQSGRIVVDYM

SEQ ID NO: 526 - protein, 100 aa, Influenza B Virus
GFFATMAWAVPKDNNKNATNPLTVEVPYVCTEGEDQITVWGFHSDNKTQMKNLYGDSNPQKFTSSANGVTTHYVSQIGGFPDQTEDGGLPQSGRIVVDYM

SEQ ID NO: 527 - protein, 100 aa, Influenza B Virus
GFFATMAWAVPRDNNKTATNPLTVEVPHICTKEEDQITVWGFHSDNKTQMKNLYGDSNPQKFTSSANGVTTHYVSQIGGFPDQTEDGGLPQSGRIVVDYM

SEQ ID NO: 528 - protein, 100 aa, Influenza B Virus
GFFATMAWAVPKDNNKNATNPLTVEVPYVCTEGEDQITVWGFHSDNKTQMKNLYGDSNPQKFTSSANGVTTHYVSQIGDFPDQTEDGGLPQSGRIVVDYM

SEQ ID NO: 529 - protein, 100 aa, Influenza B Virus; identical to SEQ ID NO: 527

SEQ ID NO: 530 - protein, 100 aa, Influenza B Virus
GFFATMAWAVPRDNNKTATNPLTVEVPHICTKEEDQITVWGFHSDNKTQMKNLYGDSNPQKFTSSANGITTHYVSQIGGFPDQTEDGGLPQSGRIVVDYM

SEQ ID NO: 531 - protein, 100 aa, Influenza B Virus; identical to SEQ ID NO: 530

SEQ ID NO: 532 - protein, 100 aa, Influenza B Virus; identical to SEQ ID NO: 474

SEQ ID NO: 533 - protein, 100 aa, Influenza B Virus; identical to SEQ ID NO: 526

SEQ ID NO: 534 - protein, 100 aa, Influenza B Virus; identical to SEQ ID NO: 474

SEQ ID NO: 535 - protein, 100 aa, Influenza B Virus; identical to SEQ ID NO: 474

SEQ ID NO: 536 - protein, 100 aa, Influenza B Virus; identical to SEQ ID NO: 474

SEQ ID NO: 537 - protein, 100 aa, Influenza B Virus
  SITE 79: note = misc_feature - Xaa can be any naturally occurring amino acid
GFFATMAWAVPKDNNKNATNPLTVEVPYICTEGEDQITVWGFHSDNKTQMKNLYGDSNPQKFTSSANGVTTHYVSQIGXFPDQTEDGGLPQSGRIVVDYM

SEQ ID NO: 538 - protein, 100 aa, Influenza B Virus
GFFATMAWAVPKDNYKNATNPLTVEVPYICTEGEDQITVWGFHSDNKTQMKNLYGDSNPQKFTSSANGVTTHYVSQIGDFPDQTEDGGLPQSGRIVVDYM

SEQ ID NO: 539 - protein, 100 aa, Influenza B Virus; identical to SEQ ID NO: 538

SEQ ID NO: 540 - protein, 100 aa, Influenza B Virus; identical to SEQ ID NO: 538

SEQ ID NO: 541 - protein, 100 aa, Influenza B Virus; identical to SEQ ID NO: 538

SEQ ID NO: 542 - protein, 100 aa, Influenza B Virus
GFFATMAWAVPKDNNKNATNPLTVEVPYICAEGEDQITVWGFHSDNKTQMKNLYGDSNPQKFTSSANGVTTHYVSQIGGFPDQTEDGGLPQSGRIVVDYM

SEQ ID NO: 543 - protein, 100 aa, Influenza B Virus; identical to SEQ ID NO: 542

SEQ ID NO: 544 - protein, 100 aa, Influenza B Virus
  SITE 22: note = misc_feature - Xaa can be any naturally occurring amino acid
GFFATMAWAVPKDNYKNATNPXTVEVPYICTEGEDQITVWGFHSDNKTQMKSLYGDSNPQKFTSSANGVTTHYVSQIGDFPDQTEDGGLPQSGRIVVDYM

SEQ ID NO: 545 - protein, 100 aa, Influenza B Virus
GFFATMAWAVPKDNYKNATNPQTVEVPYICTEGEDQITVWGFHSDNKTQMKSLYGDSNPQKFTSSANGVTTHYVSQIGDFPDQTEDGGLPQSGRIVVDYM

SEQ ID NO: 546 - protein, 100 aa, Influenza B Virus; identical to SEQ ID NO: 545

SEQ ID NO: 547 - protein, 100 aa, Influenza B Virus; identical to SEQ ID NO: 545

SEQ ID NO: 548 - protein, 100 aa, Influenza B Virus; identical to SEQ ID NO: 545

SEQ ID NO: 549 - protein, 100 aa, Influenza B Virus; identical to SEQ ID NO: 545

SEQ ID NO: 550 - protein, 100 aa, Influenza B Virus
GFFATMAWAVPKDNYKNATNPQTVEVPYICTEGEDQITVWGFHSDNKTQMKSLYGDSNPQKFTSSANGVTTHYVSQIGDFPNQTEDGGLPQSGRIVVDYM

SEQ ID NO: 551 - protein, 100 aa, Influenza B Virus
  SITE 22: note = misc_feature - Xaa can be any naturally occurring amino acid
GFFATMAWAVPKNDNNKTATNXLTIEVPYICTEGEDQITVWGFHSDNETQMAKLYGDSKPQKFTSSANGVTTHYVSQIGGFPNQTEDGGLPQSGRIVVDY

SEQ ID NO: 552 - protein, 100 aa, Influenza B Virus; identical to SEQ ID NO: 537
  SITE 79: note = misc_feature - Xaa can be any naturally occurring amino acid

SEQ ID NO: 553 - protein, 585 aa, Influenza B Virus
MKAIIVLLMVVTSNADRICTGITSSNSPHVVKTATQGEVNVTGVIPLTTTPTKSHFANLKGTETRGKLCPKCLNCTDLDVALGRPKCTGNIPSARVSILHEVRPVTSGCFPIMHDRTKIRQLPNLLRGYEHIRLSTHNVINAENAPGGPYKIGTSGSCPNVTNGNGFFATMAWAVPKNDNNKTATNSLTIEVPYICTEGEDQITVWGFHSDNEIQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 554 - protein, 585 aa, Influenza B Virus
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHIRLSTHNVINAENAPGGPYKIGTSGSCPNVTNGNGFFATMAWAVPKNDKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 555 - protein, 584 aa, Influenza B Virus
MKAIIVLLMVVTSNADRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYENIRLSTQNVIDAEKAPGGPYRLGTSGSCPNATSKIGFFATMAWAVPKDNYKNATNPLTVEVPYICKEEEDQITVWGFHSDNKTQMKNLYGDSNPQKFTSSANGVTTHYVSQIGDFPDQTEDGGLPQSGRIVVDYMMQKPGKTGTIVYQRGVLLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHTILLYYSTAASSLAVTLMLAIFIVYMVSRDNVSCSICL

SEQ ID NO: 556 - protein, 584 aa, Influenza B Virus
MKAIIVLLMVVTSNADRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTSGSCPNATSKIGFFATMAWAVPKDNYKNATNPLTVEVPYICTEGEDQITVWGFHSDNKTQMKSLYGDSNPQKFTSSANGVTTHYVSQIGDFPDQTEDGGLPQSGRIVVDYMMQKPGKTGTIVYQRGVLLPQKVWCASGRSKVIKGSLPLIGEADCLHEEYGGLNKSKPYYTGKHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHTILLYYSTAASSLAVTLMLAIFIVYMVSRDNVSCSICL